(19) United States
(12) Patent Application Publication
(10) Pub. No.: US 2022/0370642 A1
(43) Pub. Date: Nov. 24, 2022

(54) METHODS OF DETECTING NEUROLOGICAL DISORDERS VIA BINDING TO PHOSPHORYLATED TAU PROTEIN

(71) Applicant: Amydis, Inc., Los Angeles, CA (US)

(72) Inventors: Stella Sarraf, Beverly Hills, CA (US); Suhail Rasool, San Diego, CA (US)

(21) Appl. No.: 17/760,854

(22) PCT Filed: Sep. 17, 2020
(86) PCT No.: PCT/US2020/051325

§ 371 Date: Mar. 16, 2022

Related U.S. Application Data
(60) Provisional application No. 62/902,265, filed on Sep. 18, 2019.

(51) Int. Cl. A61K 49/00; A61P 25/28

(52) U.S. Cl. CPC A61K 49/0021; A61P 25/28

(57) ABSTRACT

Provided herein are methods and compositions for determining whether a patient suffers from a neurological disease or disorder is provided, comprising detecting the presence of a phosphorylated tau protein in a tissue of the patient, wherein the detecting comprises contacting the phosphorylated tau protein with a compound described herein.

### CROSS-REFERENCE TO RELATED APPLICATIONS

This application claims the benefit under 35 U.S.C. § 119(e) to U.S. Provisional Application Ser. No. 62/902,265, filed on Sep. 18, 2019 which is hereby incorporated by reference in its entirety.

### BACKGROUND

Neurological disorders are diseases of the brain, spinal cord and peripheral nervous system. The greatest societal costs, in terms of epidemiology and individual morbidity, are imposed by neurological conditions. Among the most prominent of these are Alzheimer's disease and Parkinson's disease. Other neurological conditions include age-related conditions (e.g. Parkinson's dementia, vascular dementia, Amyotrophic lateral sclerosis), genetic syndromes (e.g. Down syndrome), injury-related conditions (e.g. Traumatic Brain Injury, Chronic Traumatic Encephalopathy), and conditions typically considered as being purely psychiatric in nature, such as schizophrenia and depression.

Microtubule-associated protein tau (MAPT) are a family of proteins which stabilize microtubules during assembly and disassembly. MAPT transcripts are differentially expressed in the nervous system, depending on stage of neuronal maturation and neuron type. MAPT gene mutations have been associated with several neurological disorders such as Alzheimer's disease, Pick's disease, frontotemporal dementia, cortico-basal degeneration and progressive supranuclear palsy. Tau protein in a diseased brain, for example, of a patient having Alzheimer disease, may be abnormally phosphorylated as compared with that in the normal brain. Thus there is a need for materials and methods for detecting phosphorylated tau protein.

### SUMMARY

In some embodiments a method for determining whether a patient suffers from a neurological disease or disorder is provided, comprising detecting the presence of a phosphorylated tau protein in a tissue of the patient, wherein the detecting comprises contacting the phosphorylated tau protein with a compound described herein. The compound may be a compound of Formula I, II, Ia, Ib, Ic, Id, or Ie. In some embodiments, the compound is Compound 1, 2, 3, 4, or 5.

In some embodiments, the neurological disease or disorder is selected from an age-related disease or disorder, a genetic disease or disorder, an injury-related disease or disorder, and a psychiatric disease or disorder. In some embodiments, the age-related disease or disorder is selected from Parkinson's dementia, vascular dementia, and Amyotrophic lateral sclerosis, wherein the genetic disease or disorder is Down syndrome, wherein the injury-related disease or disorder is selected from Traumatic Brain Injury and Chronic Traumatic Encephalopathy, and wherein the psychiatric disease or disorder is selected from schizophrenia and depression. The neurological disease or disorder may be Alzheimer's disease or Chronic Traumatic Encephalopathy (CTE). The neurological disease or disorder may be a tauopathy.

In some embodiments a method for preparing a patient for diagnosis of a neurological disease or disorder is provided, comprising administering to a patient a compound described herein. The compound may be a compound of Formula I, II, Ia, Ib, Ic, Id, or Ie. In some embodiments, the compound is Compound 1, 2, 3, 4, or 5. In some embodiments, the administration is intravenous administration or is localized at the retina of the eye. In some embodiments, the administration is topical administration.

### BRIEF DESCRIPTION OF THE DRAWINGS

FIGS. 1A-1C illustrate retinal sections of 3RTau mice of different ages stained with 3RTau Ab, Compound 5 and DAPI, at 3 months (FIG. 1A), 6 months (FIG. 1B), and 12 months (FIG. 1C).

FIGS. 2A-2B illustrate 3RTau mice (12 months) retinal section stained with 3RTau Ab, Compound 5 and DAPI (FIG. 2A) and wild type mice (12 months) retinal section stained with 3RTau Ab, Compound 5 and DAPI (FIG. 2B).

FIG. 3 illustrates 3RTau mice (12 months) retinal section stained with 3RTau Ab, Compound 5 and DAPI.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The term “alkyl,” by itself or as part of another substituent, represent a straight (i.e. unbranched) or branched chain, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—O—CH₂—CH₂—O—CH₃, —CH₂—CH₂—O—CH₂—CH₂—O—CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Two or more heteroatoms may also be consecutive.

The terms “cycloalkyl” and “heterocycloalkyl,” by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, tetrahydropyran, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. Examples of heterocycloalkyl include, but are not limited to glucose, mannose, allose, altrose, gulose, idose, galactose, and talose. Examples of heterocycloalkyl include, but are not limited to:

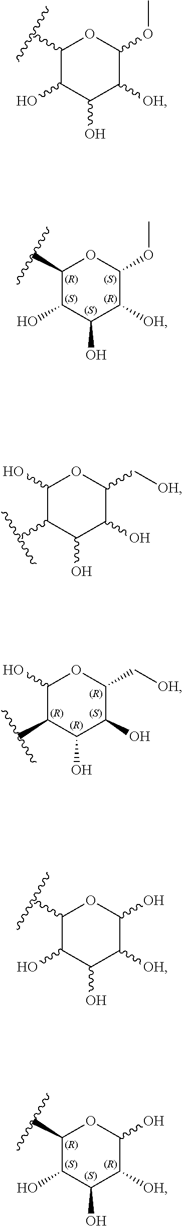

and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together (i.e. a fused ring aryl) or linked covalently. A fused ring aryl refers to multiple rings fused together wherein at least one of the fused rings is an aryl ring.

The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e. multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6, 5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, triazolyl, 1,2,3-triazolyl, 1,2,4-triazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

An “arylene” and a “heteroarylene,” alone or as part of another substituent means a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

As used herein, the term “heteroatom” or “ring heteroatom” is meant to include oxygen (O), nitrogen (N), sulfur (S), phosphorus (P), and silicon (Si).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g. arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

### Phosphorylated Tau Protein

Neurological diseases, including neurodegenerative diseases and injury-related disorders, are associated with release and/or accumulation of phosphorylated tau protein. The phosphorylated tau protein may be detected by contacting a compound as described herein, wherein the contacting, upon activation by a light, causes emission of detectable signal. Generally, the compositions and methods described herein are useful for detection of phosphorylated tau protein in any tissue of the patient. However, the phosphorylated tau protein may accumulate in the retina of a patient. Such presence of phosphorylated tau protein in the retina, as the experimental examples show, can be detected with compounds that bind to the phosphorylated tau protein, which binding can then be detected by means such as laser-activated fluorescence scanning of the retina.

“Microtubule associated protein tau,” “MAPT,” “tau protein,” or “tau” are a family of proteins which stabilize microtubules during assembly and disassembly, and are classified as microtubule-associated proteins (MAPs). Tau isoform sequences include NP_001116538.2, NP_001116539.1, NP_001190180.1, NP_001190181.1, NP_005901.2, NP_058518.1, NP_058519.3, and NP_058525.1. Tau proteins are important in the stabilization and assembly of microtubules, and in turn, affect the intraneuronal transport of cargos. Tau may also be involved in signaling pathways by interacting with actin via acidic N-terminals, projecting from microtubules for neurite outgrowth and stabilization during brain development. A tau protein as provided herein may comprise any isoform, or any combination of isoforms. MAPT transcripts are differentially expressed in the nervous system, depending on stage of neuronal maturation and neuron type. MAPT gene mutations have been associated with several neurological disorders such as Alzheimer's disease, Pick's disease, frontotemporal dementia, cortico-basal degeneration and progressive supranuclear palsy. The tau protein may or may not include post-translational modifications. The tau protein family is characterized by an N-terminal segment shared by all members, sequences of ˜50 amino acids inserted in the N-terminal segment, which are developmentally regulated in the brain, a characteristic tandem repeat region consisting of 3 or 4 tandem repeats of 31-32 amino acids, and a C-terminal tail.

The human tau gene is located on the long arm of chromosome 17 at position 17q21. The gene is believed to contain 16 exons, with exon 21 as a part of the promoter. The tau primary transcript contains 13 exons, and exons 4A, 6 and 8 are not transcribed in human. Exons 21 and 14 are transcribed but not translated. Exons 1, 4, 5, 7, 9, 11, 12, 13 are constitutive, and exons 2, 3, and 10 are alternatively spliced, giving rise to six different mRNAs, translated in six different tau isoforms. These isoforms differ by the absence or presence of one or two 29 amino acid repeat (0N, 1N, or 2N) encoded by exon 2 and 3 in the amino-terminal part, in combination with either three microtubule binding repeats (R1, R3 and R4) or four (R1-R4) repeat-regions in the carboxy-terminal part. The fourth microtubule-binding domain is encoded by exon 10. Six tau protein isoforms are known to exist in human brain tissue: (2+3+10+) isoform (having 441-amino acids), (2+3+10−) isoform (having 410-amino acids), (2+3−10+) isoform (having 412-amino acids), (2+3−10−) isoform (having 381-amino acids), (2−3−10+) isoform (having 383-amino acids), and (2−3−10−) isoform (having 352-amino acids). The tau may be a mutant tau. The mutation may be a FTDP-17 mutation. Examples of mutations include G272V, N279K, N296, P201L, P301S, G303V, S305N, L315R, S320F, P332L, V337M, E342V, S352L, K369I, G389R, R5H, R5L, K257T, I260V, L266V, G272V, delK280, N296H, N296N, delN296, P301L, P301S, K317M, G335V, Q336R, R406W and R427M.

“Phosphorylated tau protein” or “phosphorylated tau” is a tau protein having at least one amino acid residue modified by a phosphate group. Tau is believed to include as many as 85 amino acid residues compatible with phosphorylation. Generally, the phosphate group is a post-translational modification and may be bonded at a side chain of an amino acid residue. The phosphorylated amino acid residue may be, for example, a serine (S), threonine (T), or tyrosine (Y) residue, or a combination thereof. A phosphorylated tau protein may include 1, 2, 3, 4, 5, 6, 7, 8, 9, 10, 11, 12, 13, 14, 15, 16, 17, 18, 19, 20, at least 20, at least 25, at least 30, at least 40, or at least 50 moles of phosphate per mole of protein. A phosphorylated tau protein may include at least 3 moles of phosphate per mole of protein. A phosphorylated tau protein may include one or more phosphorylated amino acid residues selected from Thr39, Ser46Pro, Thr50Pro, Thr69Pro, Thr153Pro, Thr175Pro, Thr181Pro, Ser198, Ser199, Ser202Pro, Thr205Pro, Ser208, Ser210, Thr212Pro, Ser214, Thr217Pro, Thr231Pro, Ser235Pro, Ser237, Ser241, Ser262, Ser285, Ser305, Ser324, Ser352, Ser356, Ser396Pro, Ser400, Thr403, Ser404Pro, Ser409, Ser412, Ser413, Ser416, and Ser422Pro. The phosphorylated tau protein may include phosphorylated Ser422. A phosphorylated tau protein as provided herein may be aggregated or may be unaggregated. A phosphorylated tau protein as provided herein may be soluble. A tau protein, or a phosphorylated tau protein, may be a three-repeat tau, a four-repeat tau, or a combination thereof. In some embodiments, a tau protein, or a phosphorylated tau protein, may comprise a mixture of three-repeat tau and four-repeat tau in which four-repeat tau is more prevalent. In some embodiments, a tau protein, or a phosphorylated tau protein, may comprise a mixture of three-repeat tau and four-repeat tau in which three-repeat tau is more prevalent. The phosphorylated tau protein may be fibrillary, for example as a neurofibrillary tangle (NFT). The NFT may be found in the somatodendritic compartments of neurons.

In accordance with some embodiments of the present disclosure, therefore, provided is a method for determining whether a patient suffers from a neurological disease or disorder. The method entails detecting the presence of a phosphorylated tau protein in a tissue of the patient, by contacting a tissue of the patient with a compound described herein. The contacting may be in vivo or ex vivo. The contacting may be by administration to a patient, for example, by topical or intravenous administration. It is contemplated that the accumulation of the phosphorylated tau protein may occur in the retina of the eye. Accordingly, the detection can target phosphorylated tau protein in the retina.

In another embodiment, provided is a method for preparing a patient for diagnosis of a neurological disease or disorder, which method comprises administering to the patient a compound that specifically binds an phosphorylated tau protein. The compound may be administered to an eye of the patient. Once the compound is administered to the patient, its binding to the phosphorylated tau protein may be detected with any method, including those methods described herein, which binding indicates accumulation of the phosphorylated tau protein, an indication of a neurological disease or disorder.

### Phosphorylated Tau Binding Compounds

The present disclosure further provides compounds capable of binding to a phosphorylated tau. In one embodiment, the compound may be selected from those compounds described in WO 2011/072257, WO 2015/143185 or WO 2017/004560, which are incorporated by reference in their entirety.

In one embodiment, the compound can be selected from compounds described in WO 2015/143185, which compounds are described below.

In some embodiments, the disclosure provides a compound of Formula I or a salt or solvate thereof:

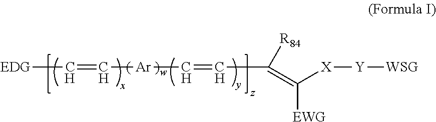

wherein

EDG is an electron donating group;

each Ar is independently C₁-C₁₄ arylene or C₁-C₁₄ heteroarylene, each optionally substituted with one or more R₁;

each R₁ is independently halogen, —OR₂, —NR₃R₄, C₁-C₁₀ alkyl, C₁-C₁₀ heteroalkyl, C₁-C₁₀ cycloalkyl, heterocycloalkyl, arylene, or C₁-C₁₀ heteroarylene;

wherein the alkyl, heteroalkyl, cycloalkyl, heterocycloalkyl, arylene, or heteroarylene is optionally substituted with one or more R₅;

R₂, R₃ and R₄ are independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₅;

each R₅ is independently halogen, —OR₆, —NR₇R₈, C₁-C₁₀ alkyl, C₁-C₁₀ heteroalkyl, C₁-C₁₀ cycloalkyl, heterocycloalkyl, arylene, or C₁-C₁₀ heteroarylene;

R₆, R₇, R₈ and R₈₄ are independently hydrogen or C₁-C₁₀ alkyl;

R₈₄ is hydrogen or C₁-C₁₀ alkyl;

EWG is an electron withdrawing group;

WSG is a water soluble group;

X is C═O or SO₂ or X and R₈₄ join to form a pyridinyl;

Y is NH, or S;

each x is independently an integer from 0-10;

each w is independently an integer from 1-5;

each y is independently an integer from 0-10; and

z is an integer from 1-10.

In some embodiments, the disclosure provides a compound of Formula II or a salt or solvate thereof:

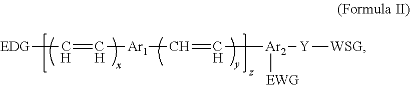

wherein

EDG is an electron donating group;

Ar₂ and each Ar₁ is independently C₁-C₁₄ arylene or C₁-C₁₄ heteroarylene;

each optionally substituted with one or more R₄₁;

each R₄₁ is independently halogen, —CN, —OR₄₂, —NR₄₃R₄₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene wherein the alkyl, heteroalkyl, cycloalkyl, heterocycloalkyl, arylene, or heteroarylene is optionally substituted with one or more R₄₅;

R₄₂, R₄₃ and R₄₄ are independently hydrogen, C₁-C₁₀ alkyl, C₁-C₁₀ heteroalkyl, cycloalkyl, C₁-C₁₀ heterocycloalkyl, C₁-C₁₀ arylene, or C₁-C₁₀ heteroarylene, each of which except for hydrogen is optionally substituted with one or more R₄₅;

each R₄₅ is independently halogen, —OR₄₆, —NR₄₇R₄₈, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and R₄₆, R₄₇ and R₄₈ are independently hydrogen or C₁-C₁₀ alkyl;

EWG is an electron withdrawing group;

Y is absent, O, NH, or S;

WSG is hydrogen or a water soluble group;

x is an integer from 0-10;

y is an integer from 0-10; and

z is an integer from 1-10.

In some embodiments, R₈₄ is hydrogen. In some embodiments, R₈₄ is C₁-C₁₀ alkyl. In some embodiments, R₈₄ is methyl, ethyl, propyl, butyl, pentyl, hexyl, heptyl, octyl, neptyl or decyl. In some embodiments, R₈₄ is methyl.

The substituent EDG is an electron donor group, as known in the art. In some embodiments, EDG is any atom or functional group that is capable of donating some of its electron density into a conjugated pi system, thus making the pi system more nucleophilic.

In some embodiments,

EDG is —OR₉, —NR₁₀R₁₁, —SR₁₂, —PR₁₃R₁₄, —NR₁₅C(O)R₁₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₁₇;

each R₁₇ is independently halogen, —ORB, —NR₁₉R₂₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;

each of R₉, R₁₀, R₁₁, R₁₂, R₁₃, R₁₄, R₁₅, R₁₆, R₁₈, R₁₉ and Rao is independently hydrogen, C₁-C₁₀ alkyl, C₁-C₁₀ heteroalkyl, C₁-C₁₀ cycloalkyl, heterocycloalkyl, arylene, or C₁-C₁₀ heteroarylene,

each of which except for hydrogen is optionally substituted with one or more R₂₁ and wherein R₁₀ and R₁₁ are optionally joined together to form a heterocycloalkyl or heteroaryl optionally substituted with R₂₁;

each of R₂₁ is independently halogen, —OR₂₂, —NR₂₃R₂₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene,

wherein the alkyl, heteroalkyl, cycloalkyl, heterocycloalkyl, arylene, or heteroarylene is optionally substituted with one or more R₂₅;

each of R₂₂, R₂₃ and R₂₄ is independently hydrogen or C₁-C₁₀ alkyl; and

each R₂₅ is independently C₁-C₁₀ alkyl, C₁-C₁₀ heteroalkyl, cycloalkyl, heterocycloalkyl, arylene, or C₁-C₁₀ heteroarylene.

In some embodiments, the EDG is selected from a group consisting of

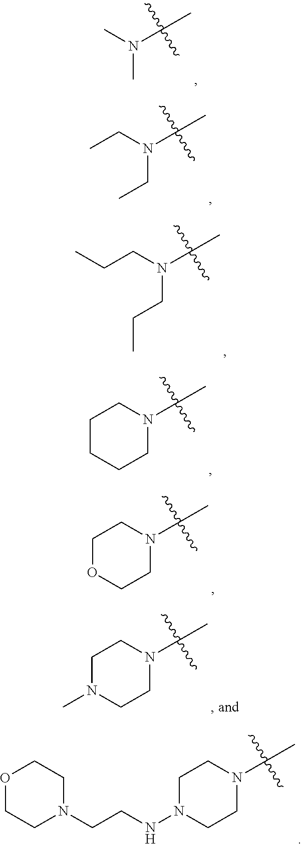

In some embodiments, the EDG is

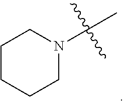 [SMILES: *N1CCCCC1 ; "."]

In some embodiments, EDG is

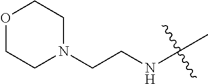 [SMILES: *NCCN1CCOCC1]

EWG is an electron withdrawing group. In some embodiments, the electron withdrawing group as used herein is any atom or group that is capable of drawing electron density from neighboring atoms towards itself, either by resonance or inductive effects.

In some embodiments,

EWG is selected from a group consisting of halogen, —CN, —NO₂, —SO₃H, —CR₂₆R₂₇R₂₈, —COR₂₉, or —COOR₃₀;

each R₂₆, R₂₇ and R₂₈ is independently hydrogen or halogen;

R₂₉ is halogen, hydrogen, C₁-C₁₀ alkyl, C₁-C₁₀ heteroalkyl, C₁-C₁₀ cycloalkyl, C₁-C₁₀ heterocycloalkyl, arylene, or C₁-C₁₀ heteroarylene,

wherein the alkyl, heteroalkyl, cycloalkyl, heterocycloalkyl, arylene, or heteroarylene is optionally substituted with one or more R₃₁;

R₃₀ is hydrogen, C₁-C₁₀ alkyl, C₁-C₁₀ heteroalkyl, cycloalkyl, heterocycloalkyl, arylene, or C₁-C₁₀ heteroarylene,

wherein the alkyl, heteroalkyl, cycloalkyl, heterocycloalkyl, arylene, or heteroarylene is optionally substituted with one or more R₃₂; and

each R₃₁ and R₃₂ is independently C₁-C₁₀ alkyl, C₁-C₁₀ heteroalkyl, cycloalkyl, C₁-C₁₀ heterocycloalkyl, C₁-C₁₀ arylene, or C₁-C₁₀ heteroarylene.

In some embodiments, the EWG is selected from a group consisting of —F, —Cl, —Br, —CH═O, NO₂, —CF₃, —CCl₃, —SO₃ and —CN. In some embodiments, the EWG is F, Cl, or Br. In some embodiments, the EWG is —CN.

WSG is a water soluble group. In some embodiments, the WSG group serves to alter the solubility of the compounds in an aqueous systems.

In some embodiments,

WSG is hydrogen, C₁-C₁₀ alkyl, C₁-C₁₀ heteroalkyl, C₁-C₁₀ cycloalkyl, C₁-C₁₀ heterocycloalkyl, arylene, or C₁-C₁₀ heteroarylene,

wherein the alkyl, heteroalkyl, cycloalkyl, heterocycloalkyl, arylene, or heteroarylene is optionally substituted with one or more R₃₃;

wherein each R₃₃ is independently halogen, —OR₃₄, —NR₃₅R₃₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene,

wherein the alkyl, heteroalkyl, cycloalkyl, heterocycloalkyl, arylene, or heteroarylene is optionally substituted with one or more R₃₇;

each R₃₄, R₃₅ and R₃₆ is independently hydrogen, C₁-C₁₀ alkyl, C₁-C₁₀ heteroalkyl, C₁-C₁₀ cycloalkyl, heterocycloalkyl, arylene, or C₁-C₁₀ heteroarylene,

wherein the alkyl, heteroalkyl, cycloalkyl, heterocycloalkyl, arylene, or heteroarylene is optionally substituted with one or more R₃₇;

each R₃₇ is independently halogen, —OR₃₈, —NR₃₉R₄₀, C₁-C₁₀ alkyl, C₁-C₁₀ heteroalkyl, C₁-C₁₀ cycloalkyl, C₁-C₁₀ heterocycloalkyl, —(C₁-C₆alkyl)(C₁-C₁₀heretocycloalkyl), C₁-C₁₀ arylene, or C₁-C₁₀ heteroarylene; and

each of R₃₈, R₃₉ and R₄₀ is independently hydrogen or C₁-C₁₀ alkyl.

In some embodiments, the WSG is

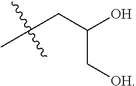 [SMILES: *CC(O)CO]

In some embodiments, WSG is polyethylene glycol, polypropylene glycol, co-polymer of polyethylene glycol and polypropylene glycol, or alkoxy derivatives thereof.

In some embodiments, WSG is

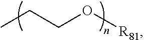

wherein n is an integer from 1-50 and R₈₁ is hydrogen, C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, heterocycloalkyl, arylene, or C₁-C₁₀ heteroarylene. In some embodiments, R₈₁ is hydrogen. In some embodiments, R₈₁ is methyl. In some embodiments, R₈₁ is ethyl. In some embodiments, R₈₁ is —CH₂—C≡CH. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, n is an integer of value 2-10, 1-10, 1-20, 1-30, 1-40, 1-50, 10-20, 10-30, 10-40, 10-50, 20-30, 20-40, 20-50, 30-40, 30-50, or 40-50. In some embodiments, n is 1, 2, 3, 4, 5, 6, 7, 8, 9, or 10. In some embodiments, and n is 3 or 6.

In some embodiments, WSG is

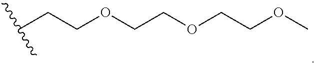 [SMILES: *CCOCCOCCOC ; "."]

In some embodiments, WSG is

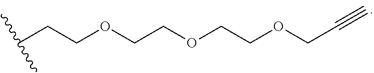 [SMILES: *CCOCCOCCOCC#C]

In some embodiments, the WSG is

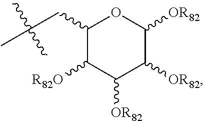

wherein each R₈₂ is independently hydrogen or C₁-C₁₀ alkyl.

In some embodiments, each R₈₂ is independently a hydrogen, methyl, ethyl, propyl, or butyl.

In some embodiments, the WSG is

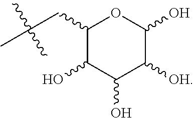 [SMILES: *CC1OC(O)C(O)C(O)C1O]

In some embodiments, the WSG is

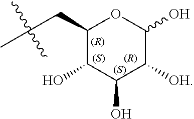

In some embodiments, the WSG is

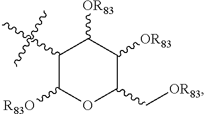

wherein each R₈₃ is hydrogen or C₁-C₁₀ alkyl. In some embodiments, each R₈₃ is independently a hydrogen, methyl, ethyl, propyl, or butyl.

In some embodiments, the WSG is

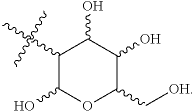

In some embodiments, the WSG is

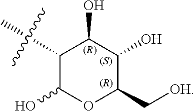

In some embodiments, WSG is —(C₁-C₁₀ alkylene)-R₃₃-R₃₇. In some embodiments, WSG is —(C₁-C₁₀ alkylene)-R₃₃-R₃₇ and R₃₃ is C₁-C₁₀ heteroarylene. In some embodiments, WSG is alkyl)-R₃₃-R₃₇, R₃₃ is C₁-C₁₀ heteroarylene and R₃₇ is —(C₁-C₆alkyl)(C₁-C₁₀heretocycloalkyl). In some embodiments, WSG is —CH₂-R₃₃-R₃₇. In some embodiments, WSG is —CH₂-R₃₃-R₃₇ and R₃₃ is triazole, imidazole, or pyrazole. In some embodiments, WSG is —CH₂-R₃₃-R₃₇ and R₃₃ is triazole. In some embodiments, WSG is —CH₂-R₃₃-R₃₇ and R₃₃ is 1,2,4-triazole. In some embodiments, WSG is —CH₂-R₃₃-R₃₇ and R₃₃ is 1,2,3-triazole. In some embodiments, WSG is —CH₂-R₃₃-R₃₇, R₃₃ is 1,2,3-triazole and R₃₇ is —(C₁-C₆alkyl)(C₁-C₁₀heretocycloalkyl). In some embodiments, WSG is —CH₂-R₃₃-R₃₇, R₃₃ is 1,2,3-triazole and R₃₇ is —(C₁alkyl)(C₁-C₁₀heretocycloalkyl). In some embodiments, WSG is —CH₂-R₃₃-R₃₇, R₃₃ is 1,2,3-triazole, R₃₇ is —(C₁alkyl)(C₁-C₁₀heretocycloalkyl), and C₁-C₁₀heretocycloalkyl is a tetrahydropyran derivative.

In some embodiments, WSG is

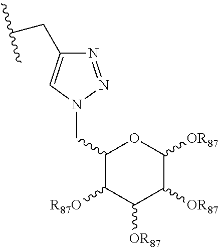

wherein each R₈₇ is hydrogen, C₁-C₁₀ alkyl, or —C(═O)C₁-C₁₀ alkyl. In some embodiments, each R₈₇ is independently a hydrogen, methyl, ethyl, propyl, butyl, acetate, propionate, or butyrate. In some embodiments, each R₈₇ is independently a hydrogen or methyl. In some embodiments, each R₈₇ is independently a methyl or acetate.

In some embodiments, WSG is

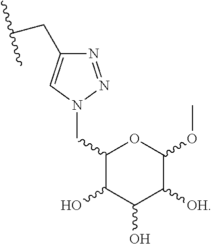 [SMILES: *Cc1cn(CC2OC(OC)C(O)C(O)C2O)nn1]

In some embodiments, WSG is

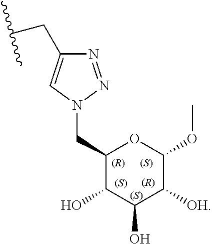

In some embodiments, WSG is —(C₁-C₁₀ heteroalkyl)-R₃₃-R₃₇. In some embodiments, WSG is —(C₁-C₁₀ heteroalkyl)-R₃₃-R₃₇ and R₃₃ is C₁-C₁₀ heteroarylene. In some embodiments, WSG is —(C₁-C₁₀ heteroalkyl)-R₃₃-R₃₇ and R₃₃ is C₁-C₁₀ heteroarylene and R³⁷ is —(C₁-C₆alkyl)(C₁-C₁₀heretocycloalkyl).

In some embodiments, WSG is

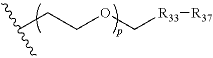

and p is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, p is an integer of value 2-10, 1-10, 1-20, 1-30, 1-40, 1-50, 10-20, 10-30, 10-40, 10-50, 20-30, 20-40, 20-50, 30-40, 30-50, or 40-50. In some embodiments, p is 1, 2, 3, 4, 5, 6, 7, 8, 9, or 10. In some embodiments, p is 3 or 6. In some embodiments, p 3.

In some embodiments, WSG is

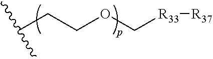

and R₃₃ is a C₁-C₁₀ heteroarylene. In some embodiments, R₃₃ is a C₅ heteroarylene. In some embodiments, R₃₃ is triazole, imidazole, or pyrazole. In some embodiments, R₃₃ is triazole. In some embodiments, R₃₃ is 1, 2, 4-triazole. In some embodiments, R₃₃ is 1, 2, 3-triazole. In some embodiments, R₃₃ is 1, 2, 3-triazole, and p is 3. In some embodiments, R₃₃ is 1,2,3-triazole and R₃₇ is —(C₁-C₆alkyl)(C₁-C₁₀heretocycloalkyl). In some embodiments, R₃₃ is 1,2,3-triazole and R₃₇ is —(C₁alkyl)(C₁-C₁₀heretocycloalkyl). In some embodiments, R₃₃ is 1,2,3-triazole, R₃₇ is a tetrahydropyran derivative. In some embodiments, R₃₃ is 1,2,3-triazole, and R₃₇ is

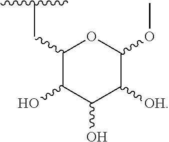 [SMILES: *CC1OC(OC)C(O)C(O)C1O]

In some embodiments, WSG is

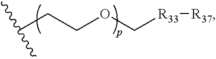

R₃₃ is 1,2,3-triazole, R₃₇ is

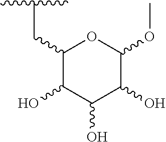 [SMILES: *CC1OC(OC)C(O)C(O)C1O]

and p is 3.

In some embodiments, WSG is

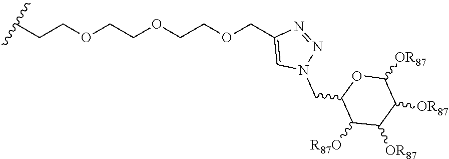

wherein each R₈₇ is hydrogen, C₁-C₁₀ alkyl, or —C(═O)C₁-C₁₀ alkyl. In some embodiments, each R₈₇ is independently a hydrogen, methyl, ethyl, propyl, butyl, acetate, propionate, or butyrate. In some embodiments, each R₈₇ is independently a hydrogen or methyl. In some embodiments, each R₈₇ is independently a methyl or acetate.

In some embodiments, WSG is

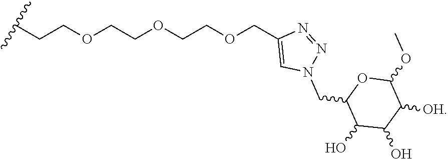 [SMILES: *CCOCCOCCOCc1cn(CC2OC(OC)C(O)C(O)C2O)nn1]

In some embodiments, WSG is

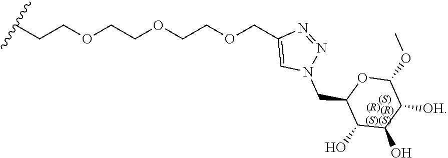

In some embodiments, X is C═O or SO₂. In some embodiments, X is C═O. In some embodiments, X is SO₂.

In some embodiments, Y is NH or S. In some embodiments, Y is NH. In some embodiments, Y is S.

The variable win Formula I is an integer from 1-5. In some embodiments, w is 1. In some embodiments, w is 2. In some embodiments, w is 3. In some embodiments, w is 4. In some embodiments, w is 5.

The variable x in Formula I is an integer from 0-10. In some embodiments, x is 0. In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10.

The variable y in Formula I is an integer from 0-10. In some embodiments, y is 0. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10.

The variable z in Formula I is an integer from 1-10. In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10.

In some embodiments, x is 0, w is 1, y is 0, z is 1, X is C═O, and Y is NH.

In some embodiments, x is 0, w is 1, y is 0, z is 1, X is SO₂, and Y is NH.

In some embodiments, x is 0, w is 2, y is 0, z is 1, X is C═O, and Y is NH.

In some embodiments, x is 0, w is 2, y is 0, z is 1, X is SO₂, and Y is NH.

In some embodiments, the disclosure provides a compound of Formula Ia or a salt or solvate thereof:

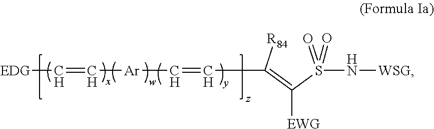

wherein EDG, Ar, R₈₄, x, w, y, z, EWG, and WSG are defined as above.

In some embodiments, the disclosure provides a compound of Formula Ib or a salt or solvate thereof:

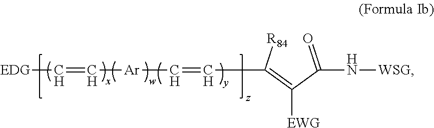

wherein EDG, Ar, R₈₄, x, w, y, z, EWG, and WSG are defined above.

In some embodiments, the disclosure provides a compound of Formula Ic or a salt or solvate thereof:

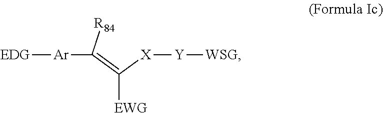

wherein EDG, Ar, R₈₄, X, Y, EWG, and WSG are defined above.

In some embodiments, the disclosure provides a compound of Formula Ic or a salt or solvate thereof:

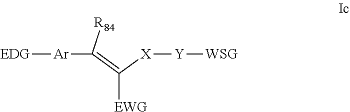

wherein

EDG is:

a) heterocycloalkyl of no more than 10 carbons optionally substituted with one or more R₁₇; or

b) —NR₁₀R₁₁;

wherein each R₁₇ is independently halogen, —OR₁₈, —NR₁₉R₂₀, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;

each of R₁₀, R₁₁, R₁₈, R₁₉ and R₂₀ is independently hydrogen, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, each of which except for hydrogen is optionally substituted with one or more R₂₁;

each of R₂₁ is independently halogen, —OR₂₂, —NR₂₃R₂₄, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, wherein the alkyl, heteroalkyl, cycloalkyl, heterocycloalkyl, arylene or heteroarylene is optionally substituted with one or more Res;
each of R₂₂, R₂₃ and R₂₄ is independently hydrogen or C₁-C₁₀ alkyl; and

each of R₂₅ is independently C₁-C₁₀ alkyl, C₁-C₁₀ heteroalkyl, cycloalkyl of no more than 10 carbons, heterocycloalkyl of no more than 10 carbons, arylene of no more than 10 carbons or heteroarylene of no more than 10 carbons;

Ar is arylene of no more than 14 carbon atoms or heteroarylene of no more than 14 carbon atoms, each optionally substituted with one or more R₁;
each R₁ is independently halogen, —OR₂, —NR₃R₄, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons wherein the alkyl, heteroalkyl, cycloalkyl, heterocycloalkyl, arylene, or heteroarylene is optionally substituted with one or more R₅;
R₂, R₃ and R₄ are independently hydrogen, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, each of which except for hydrogen is optionally substituted with one or more R₅;
   each R₅ is independently halogen, —OR₆, —NR₇R₈, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;
   R₆, R₇, R₈ and R₈₄ are independently hydrogen or C₁-C₁₀ alkyl;

EWG is selected from a group consisting of —F, —Cl, —Br, —CH═O, NO₂, —CF₃, —CCl₃, —SO₃H and —CN;

WSG is:

i)

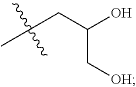 [SMILES: *CC(O)CO]

ii) polyethylene glycol, polypropylene glycol, co-polymer of polyethylene glycol and polypropylene glycol, or alkoxy derivatives thereof;

iii)

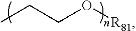

wherein n is an integer from 1-50 and R₈₁ is hydrogen, a C₁-C₁₀ alkyl, a C₁-C₁₀ alkenyl, or a C₁-C₁₀ alkynyl, wherein the alkyl, alkenyl, or alkynyl is optionally substituted with one or more C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;

iv)

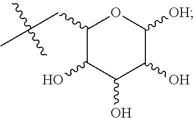 [SMILES: *CC1OC(O)C(O)C(O)C1O]

v)

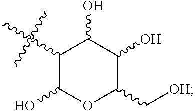

vi)
—(C₁-C₁₀ alkyl)-R₃₃-R₃₇, wherein:
R³³ is heteroarylene of no more than 10 carbons; and
R₃₇ is —(C₁-C₆alkyl)(heterocycloalkyl of no more than 10 carbons);

vii)

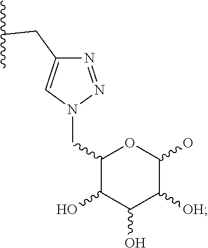 [SMILES: *Cc1cn(CC2OC(O)C(O)C(O)C2O)nn1]

viii)
—(C₁-C₁₀ heteroalkyl)-R₃₃-R₃₇, wherein:
R₃₃ is heteroarylene of no more than 10 carbons; and
R₃₇ is —(C₁-C₆alkyl)(heterocycloalkyl of no more than 10 carbons); or

ix)

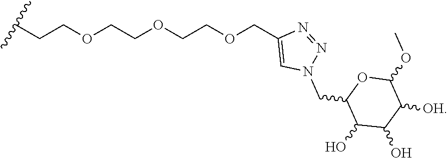 [SMILES: *CCOCCOCCOCc1cn(CC2OC(OC)C(O)C(O)C2O)nn1]

X is C═O or SO₂ or X and R₈₄ join to form a pyridinyl;

Y is NH or S.

In some embodiments, the disclosure provides a compound of Formula Id or a salt or solvate thereof:

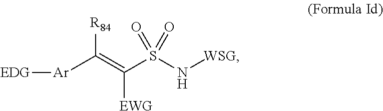

wherein EDG, R₈₄, Ar, EWG, and WSG are defined as above.

In some embodiments, the disclosure provides a compound of Formula Ie or a salt or solvate thereof:

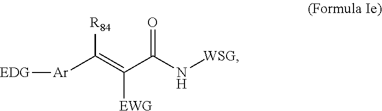

wherein EDG, R₈₄, Ar, EWG, and WSG are defined as above.

In some embodiments, the compound is selected from a group consisting of

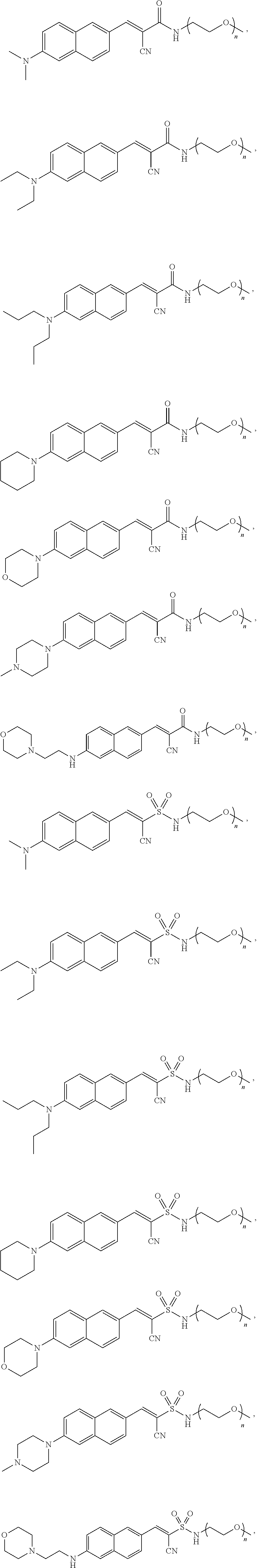

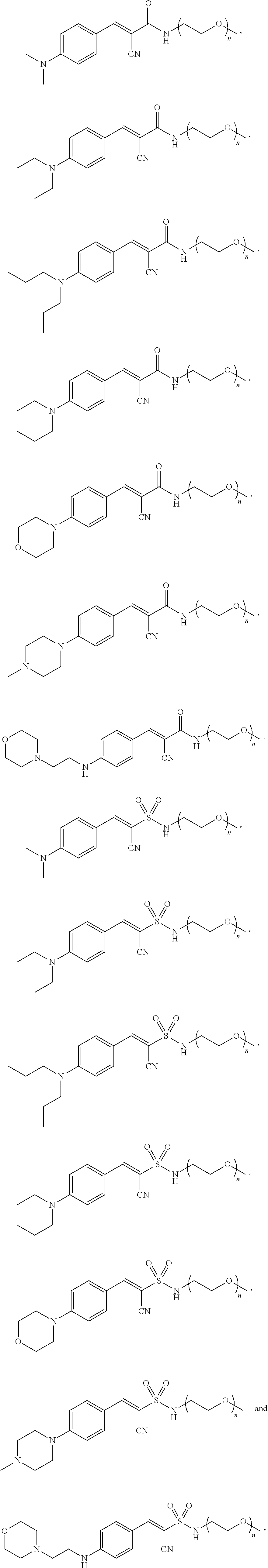

wherein n is an integer with value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

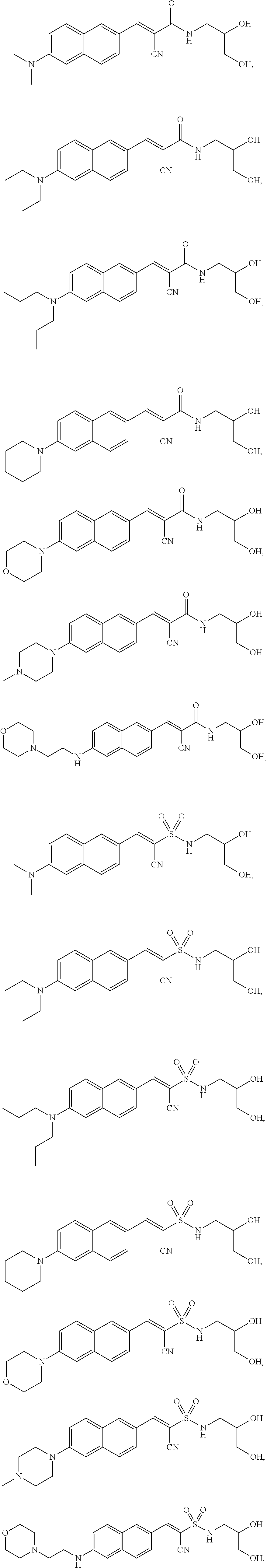

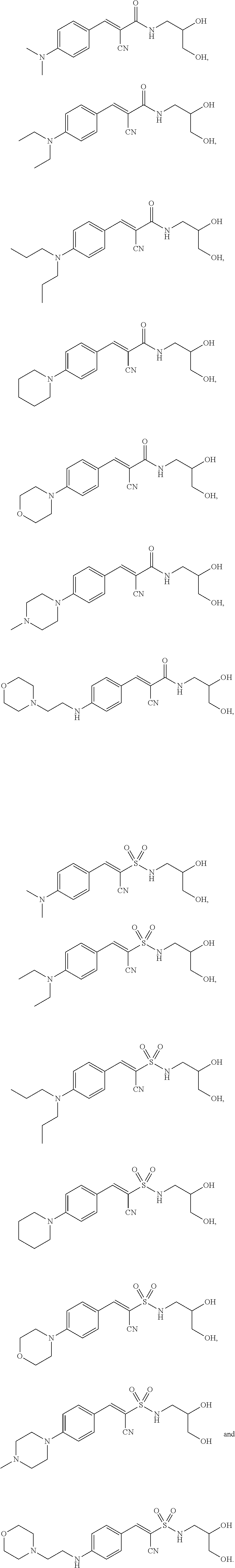

In some embodiments, the compound is selected from a group consisting of

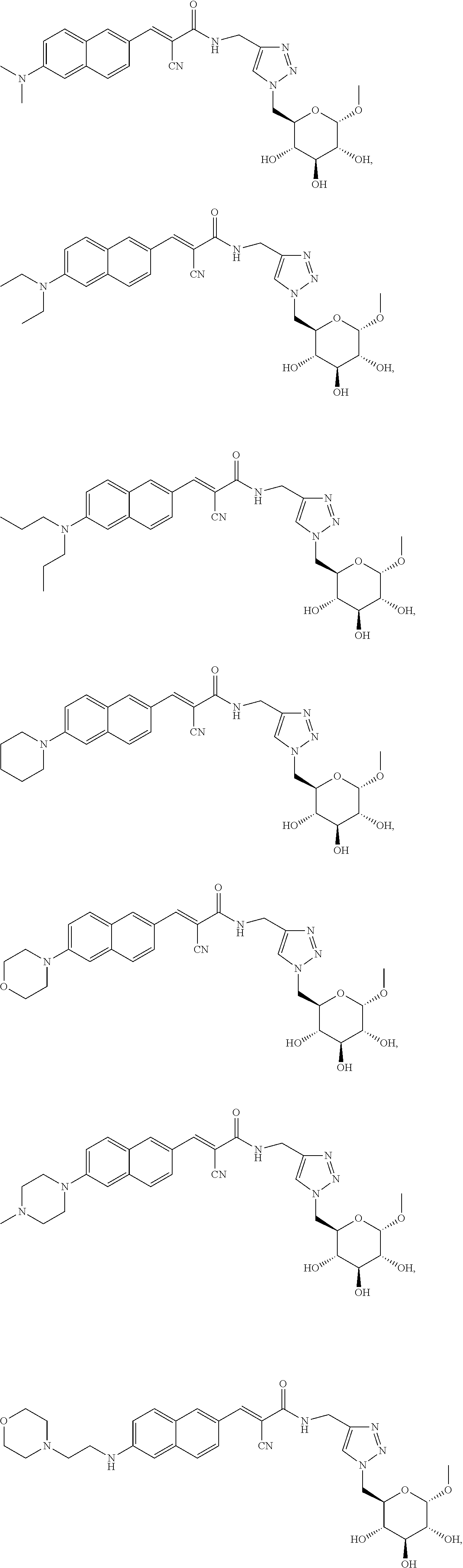

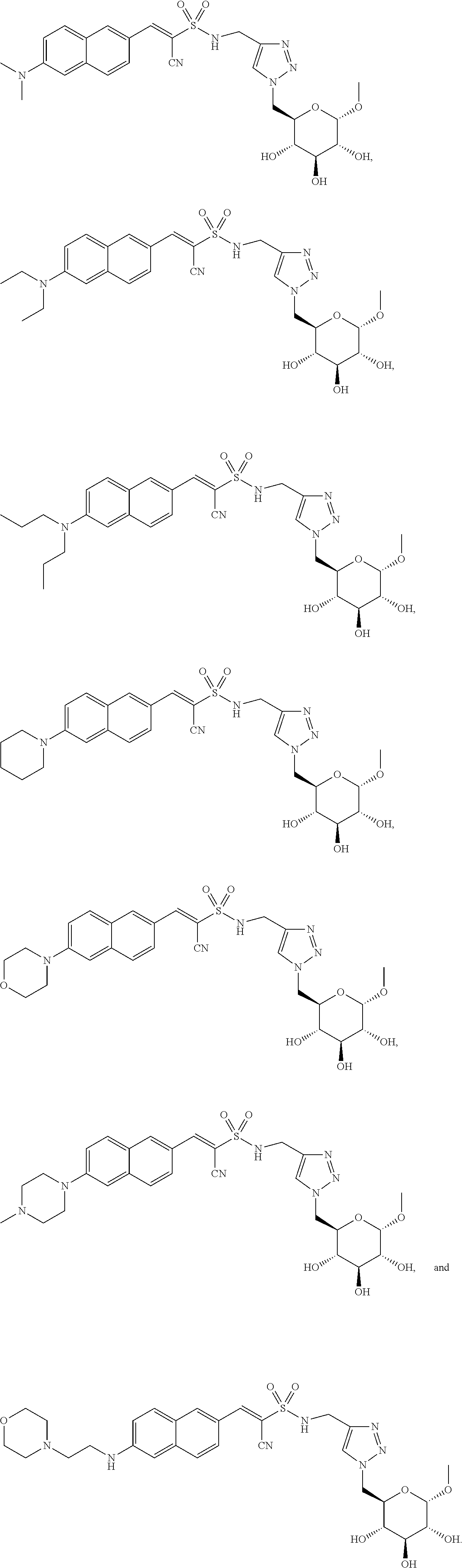

In some embodiments, the compound is selected from a group consisting of

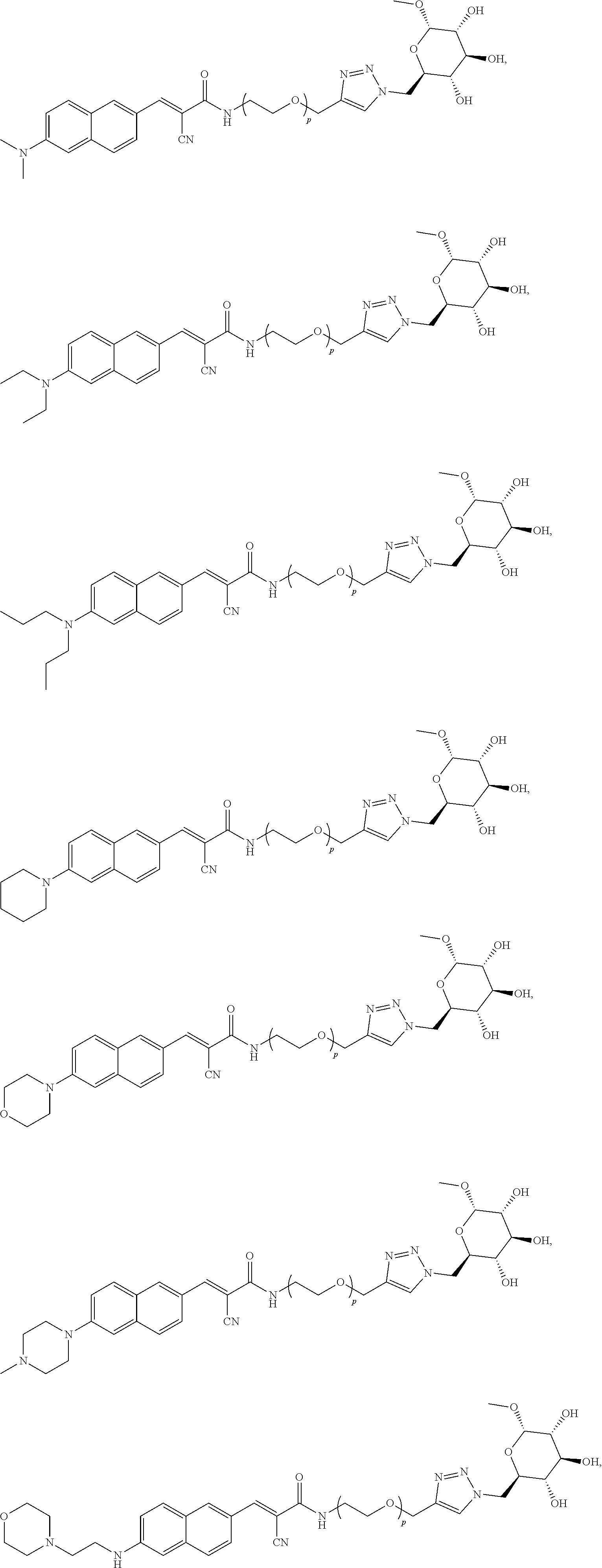

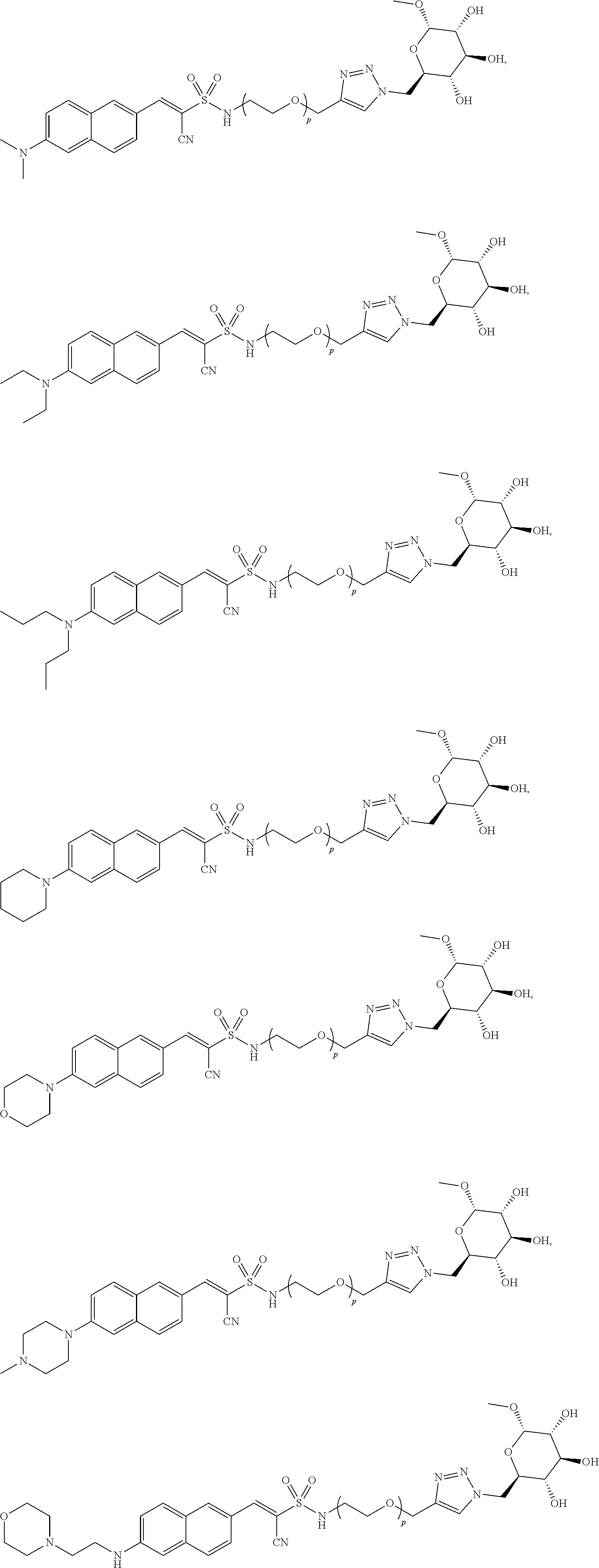

wherein p is an integer with value 1-50. In some embodiments, p is a integer of value 1-10, e.g. p is 1, 2, 3, 4, 5, 6, 7, 8, 9, or 10. In some embodiments, p is a integer of value 2-10, e.g. p is 2, 3, 4, 5, 6, 7, 8, 9, or 10.

In some embodiments, the compound is selected from a group consisting of

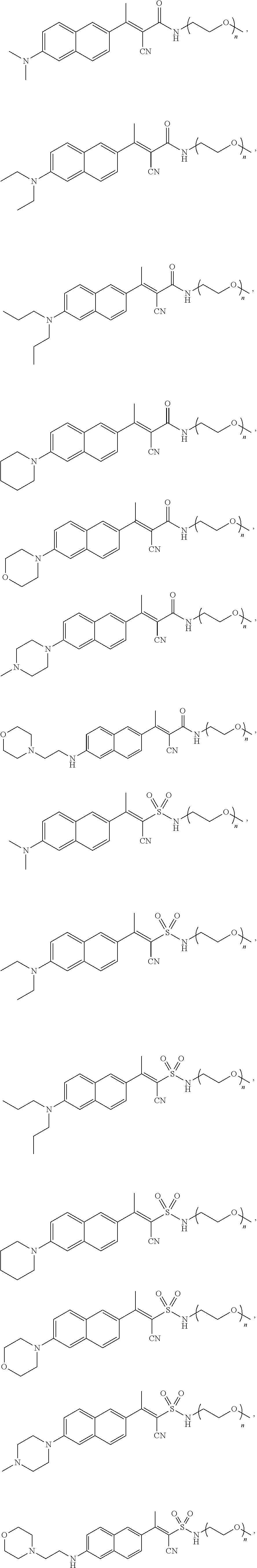

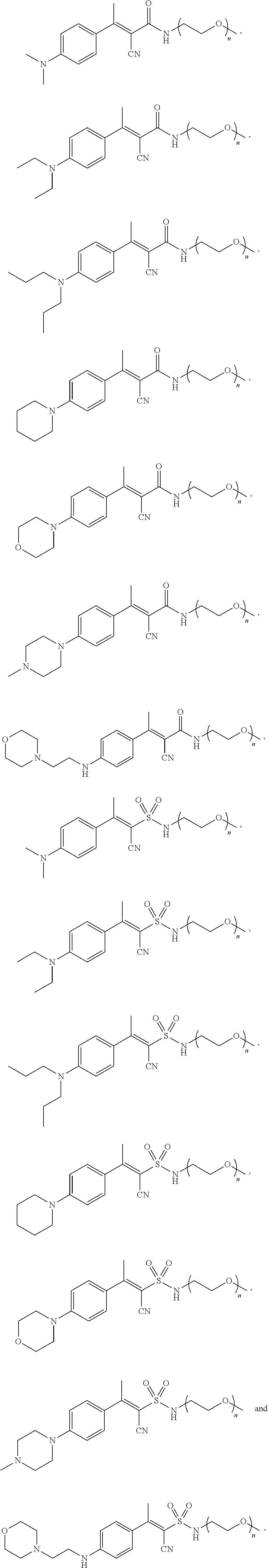

wherein n is an integer with value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

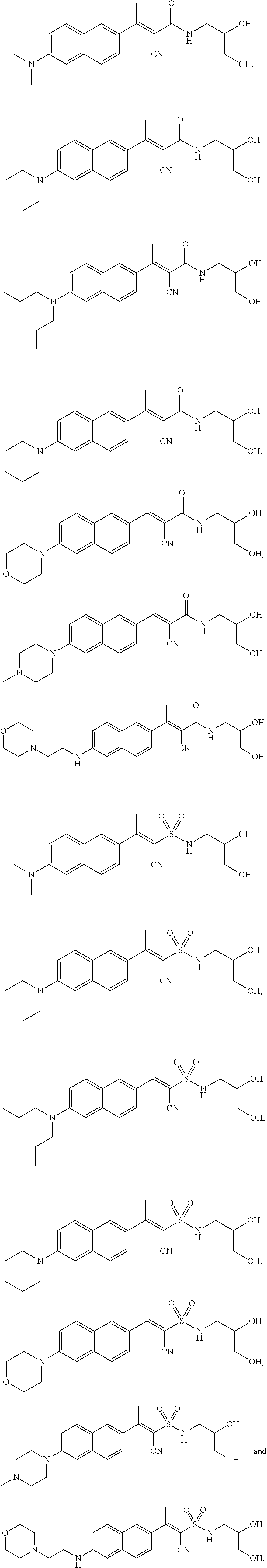

In some embodiments, the compound is selected from a group consisting of

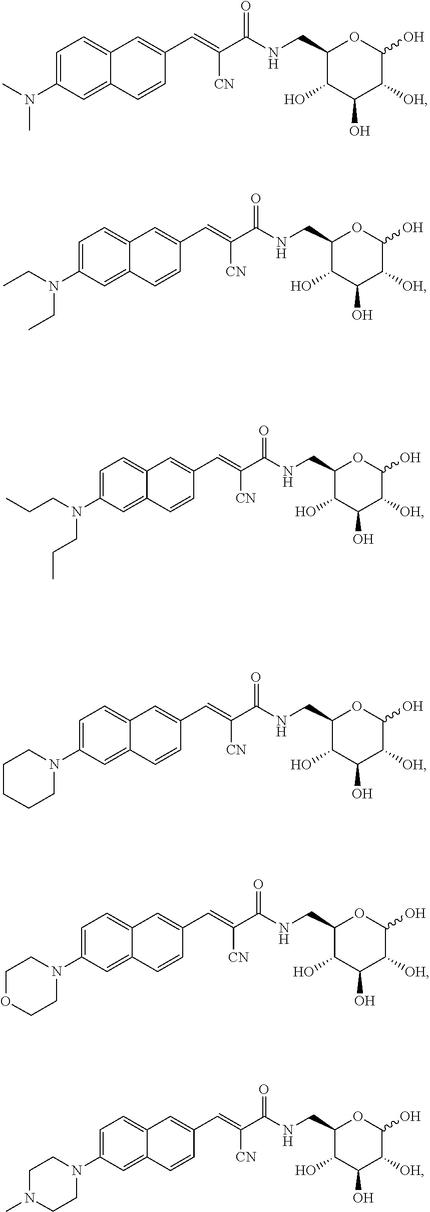 [SMILES: CN(C)c1ccc2cc(/C=C(\C#N)C(=O)NC[C@H]3OC(O)[C@H](O)[C@@H](O)[C@@H]3O)ccc2c1 ; CCN(CC)c1ccc2cc(/C=C(\C#N)C(=O)NC[C@H]3OC(O)[C@H](O)[C@@H](O)[C@@H]3O)ccc2c1 ; CCCN(CCC)c1ccc2cc(/C=C(\C#N)C(=O)NC[C@H]3OC(O)[C@H](O)[C@@H](O)[C@@H]3O)ccc2c1 ; N#C/C(=C\c1ccc2cc(N3CCCCC3)ccc2c1)C(=O)NC[C@H]1OC(O)[C@H](O)[C@@H](O)[C@@H]1O ; N#C/C(=C\c1ccc2cc(N3CCOCC3)ccc2c1)C(=O)NC[C@H]1OC(O)[C@H](O)[C@@H](O)[C@@H]1O ; CN1CCN(c2ccc3cc(/C=C(\C#N)C(=O)NC[C@H]4OC(O)[C@H](O)[C@@H](O)[C@@H]4O)ccc3c2)CC1]

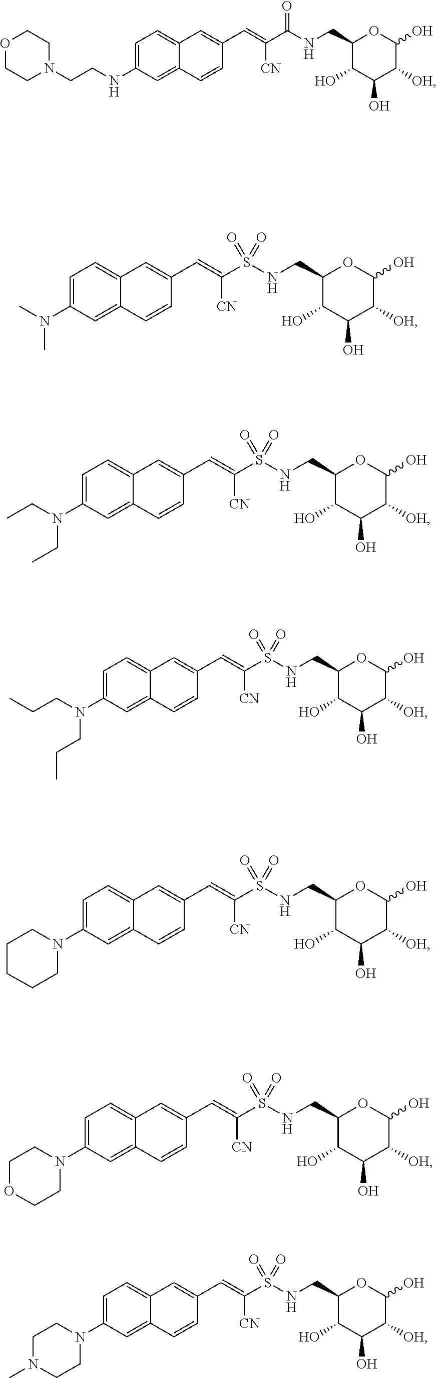

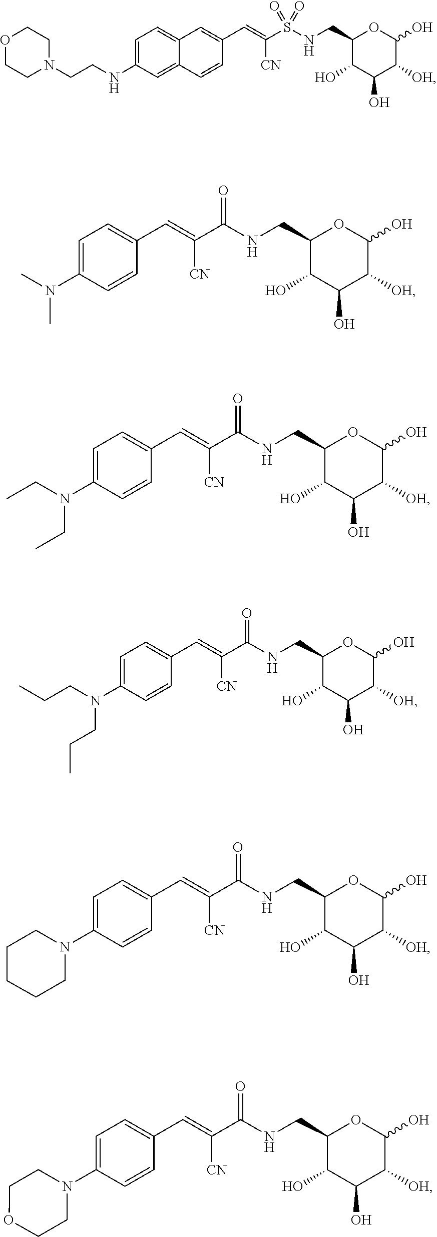 [SMILES: N#C/C(=C\c1ccc2cc(NCCN3CCOCC3)ccc2c1)S(=O)(=O)NC[C@H]1OC(O)[C@H](O)[C@@H](O)[C@@H]1O ; CN(C)c1ccc(/C=C(\C#N)C(=O)NC[C@H]2OC(O)[C@H](O)[C@@H](O)[C@@H]2O)cc1 ; CCN(CC)c1ccc(/C=C(\C#N)C(=O)NC[C@H]2OC(O)[C@H](O)[C@@H](O)[C@@H]2O)cc1 ; CCCN(CCC)c1ccc(/C=C(\C#N)C(=O)NC[C@H]2OC(O)[C@H](O)[C@@H](O)[C@@H]2O)cc1 ; N#C/C(=C\c1ccc(N2CCCCC2)cc1)C(=O)NC[C@H]1OC(O)[C@H](O)[C@@H](O)[C@@H]1O ; N#C/C(=C\c1ccc(N2CCOCC2)cc1)C(=O)NC[C@H]1OC(O)[C@H](O)[C@@H](O)[C@@H]1O]

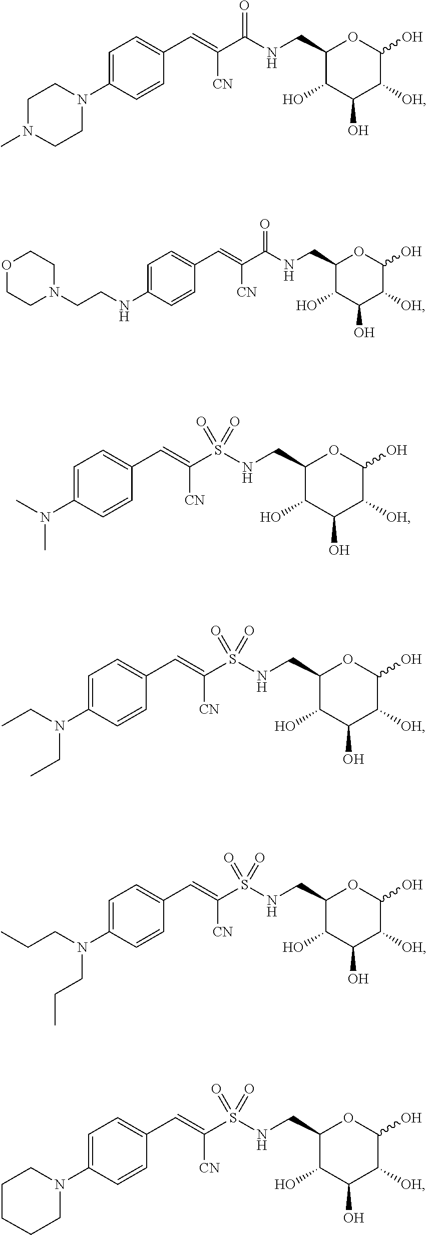 [SMILES: CN1CCN(c2ccc(/C=C(\C#N)C(=O)NC[C@H]3OC(O)[C@H](O)[C@@H](O)[C@@H]3O)cc2)CC1 ; N#C/C(=C\c1ccc(NCCN2CCOCC2)cc1)C(=O)NC[C@H]1OC(O)[C@H](O)[C@@H](O)[C@@H]1O ; CN(C)c1ccc(/C=C(\C#N)S(=O)(=O)NC[C@H]2OC(O)[C@H](O)[C@@H](O)[C@@H]2O)cc1 ; CCN(CC)c1ccc(/C=C(\C#N)S(=O)(=O)NC[C@H]2OC(O)[C@H](O)[C@@H](O)[C@@H]2O)cc1 ; CCCN(CCC)c1ccc(/C=C(\C#N)S(=O)(=O)NC[C@H]2OC(O)[C@H](O)[C@@H](O)[C@@H]2O)cc1 ; N#C/C(=C\c1ccc(N2CCCCC2)cc1)S(=O)(=O)NC[C@H]1OC(O)[C@H](O)[C@@H](O)[C@@H]1O]

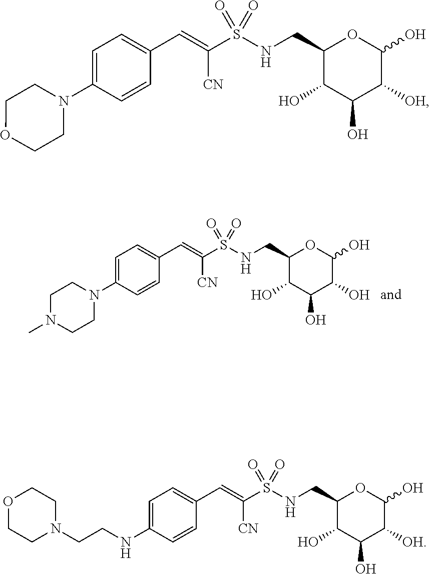 [SMILES: N#C/C(=C\c1ccc(N2CCOCC2)cc1)S(=O)(=O)NC[C@H]1OC(O)[C@H](O)[C@@H](O)[C@@H]1O ; CN1CCN(c2ccc(/C=C(\C#N)S(=O)(=O)NC[C@H]3OC(O)[C@H](O)[C@@H](O)[C@@H]3O)cc2)CC1 ; N#C/C(=C\c1ccc(NCCN2CCOCC2)cc1)S(=O)(=O)NC[C@H]1OC(O)[C@H](O)[C@@H](O)[C@@H]1O]

In some embodiments, the compound is selected from a group consisting of

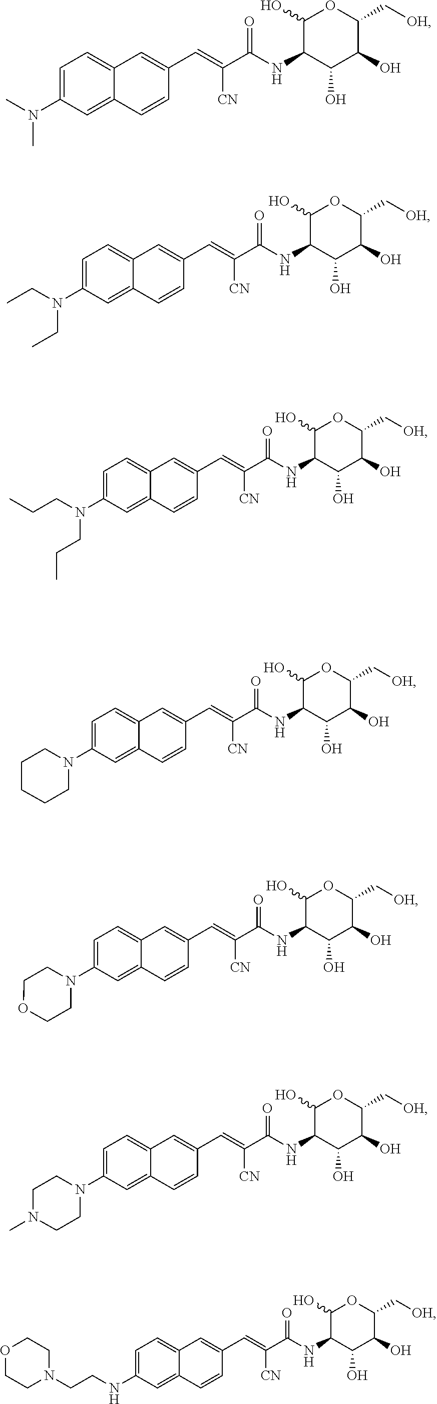

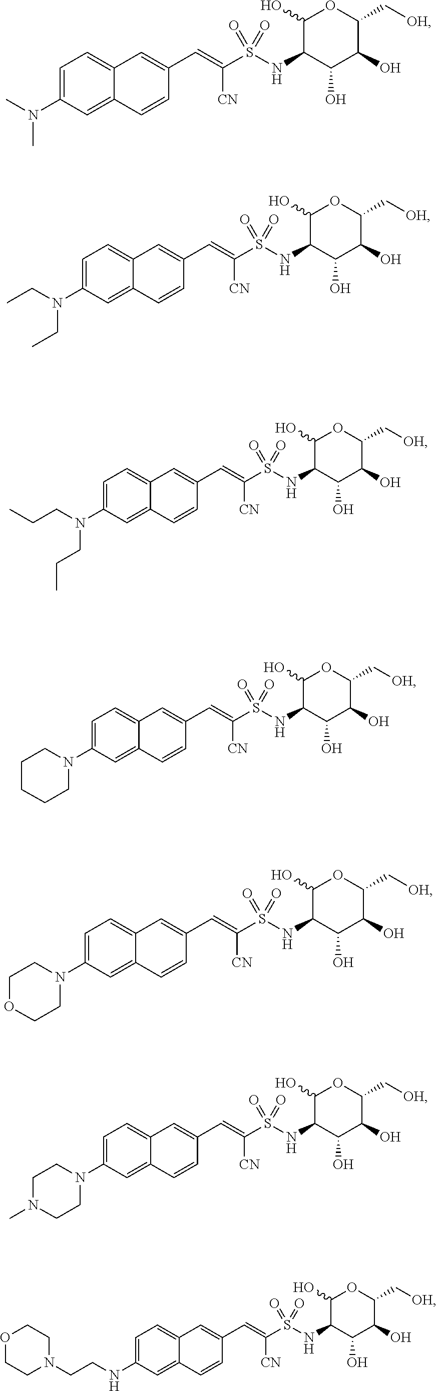

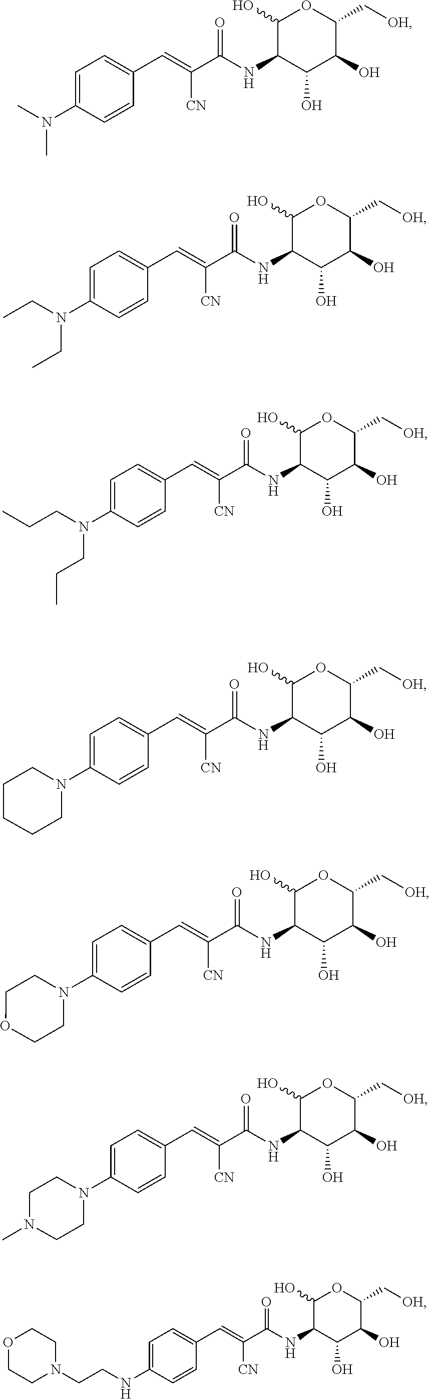

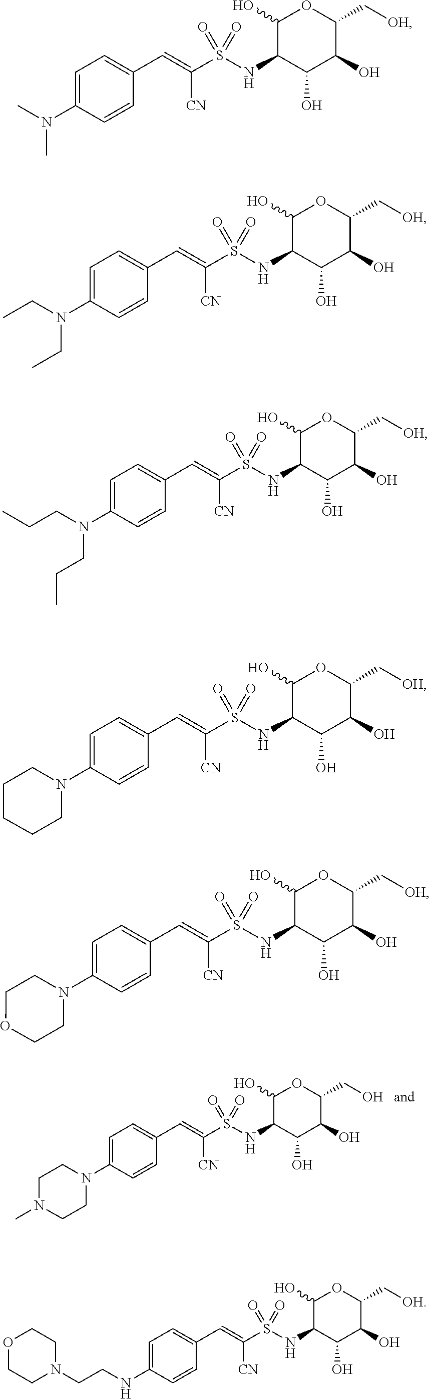

In some embodiments, the compound is selected from a group consisting of

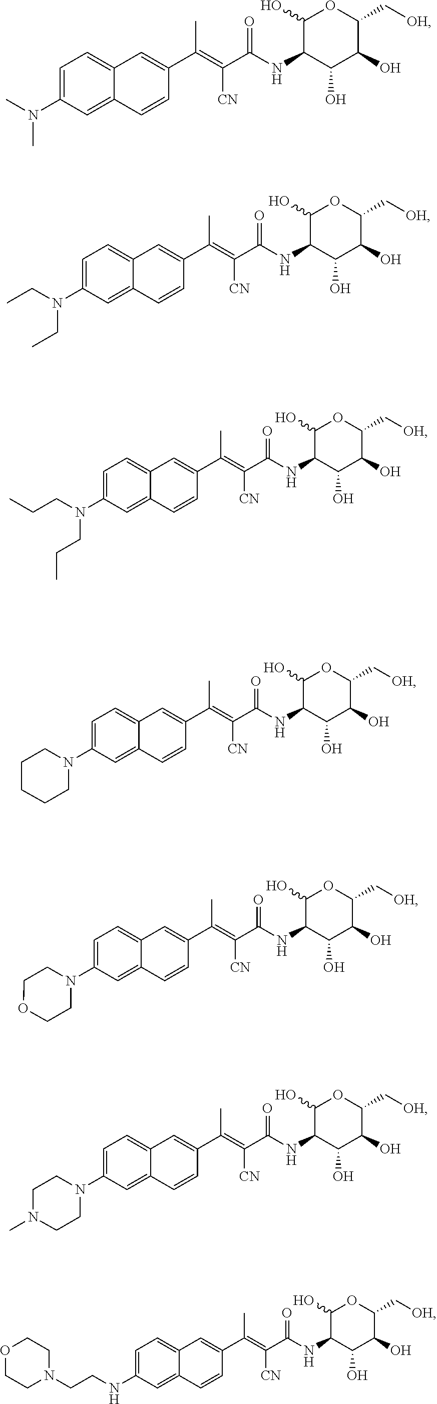

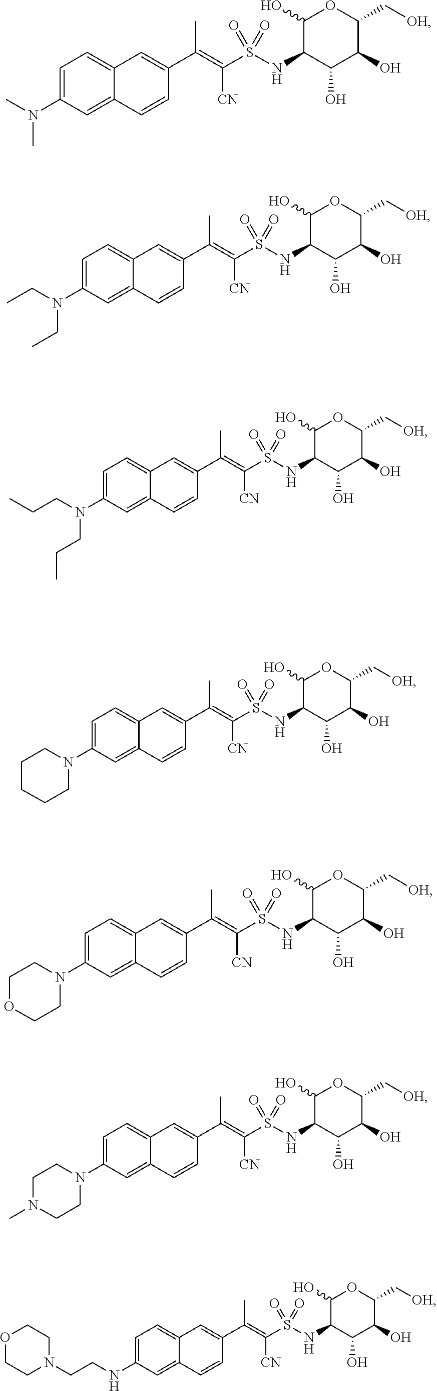

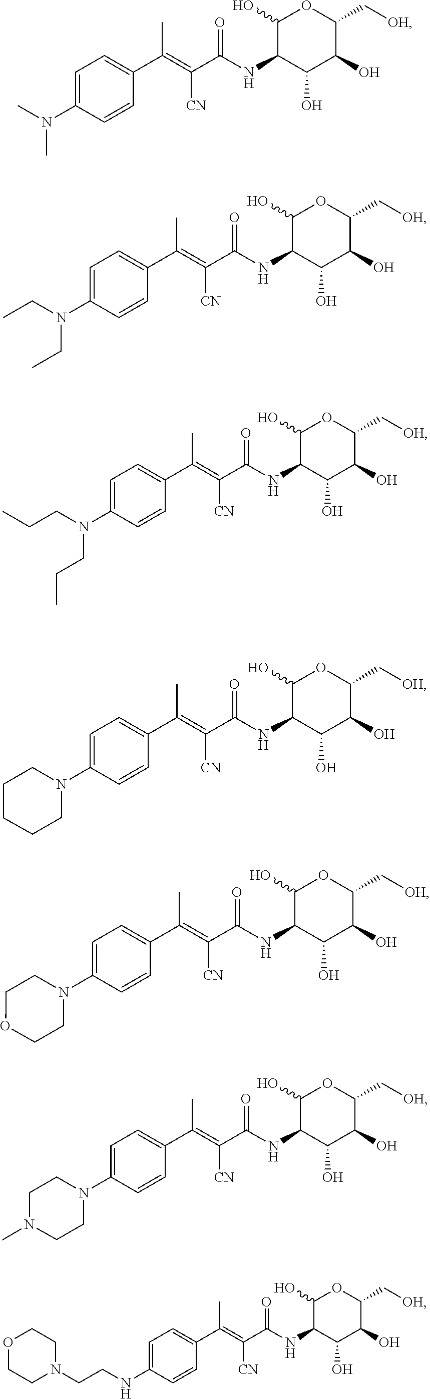

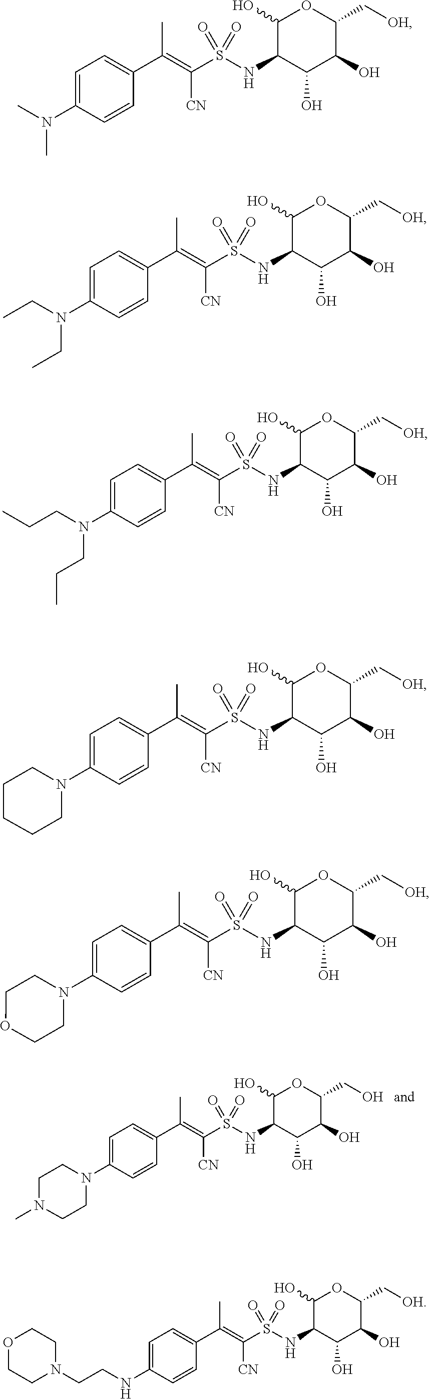

In some embodiments, the compound is selected from a group consisting of

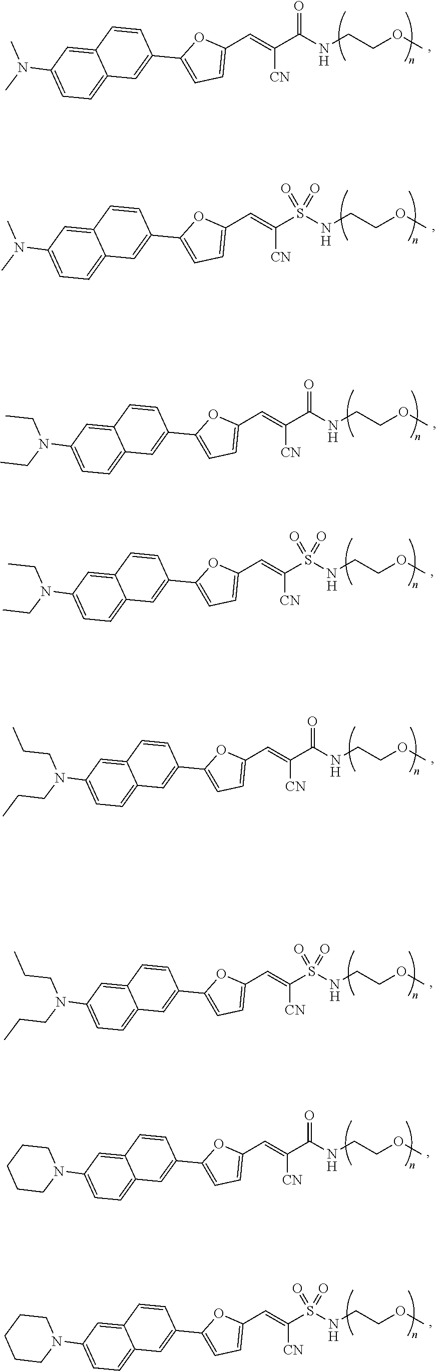

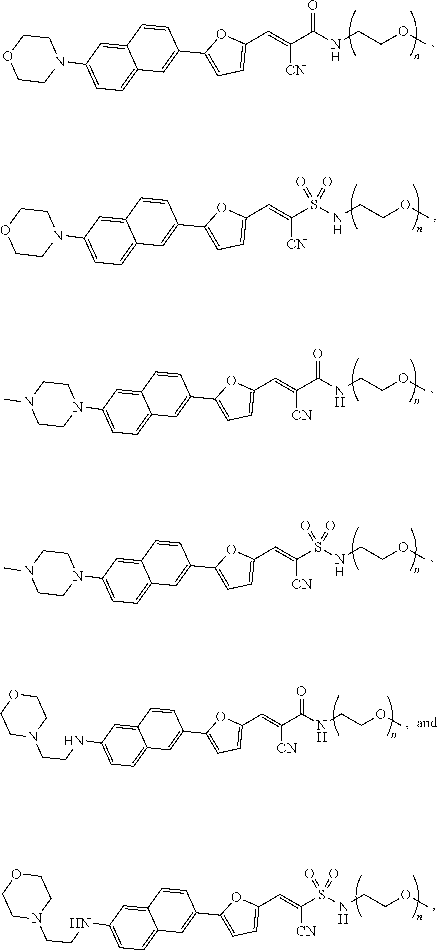

wherein n is an integer with value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

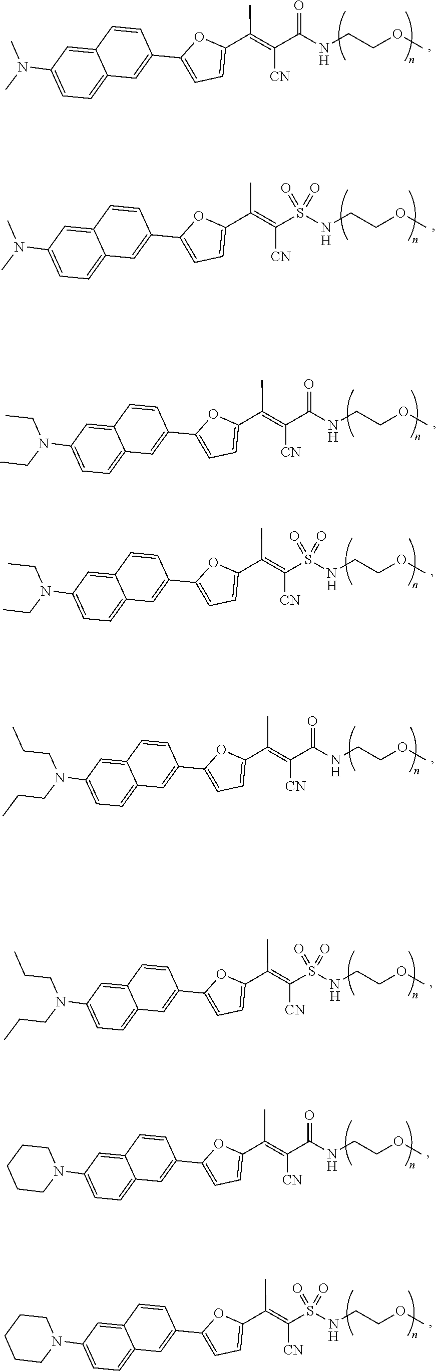

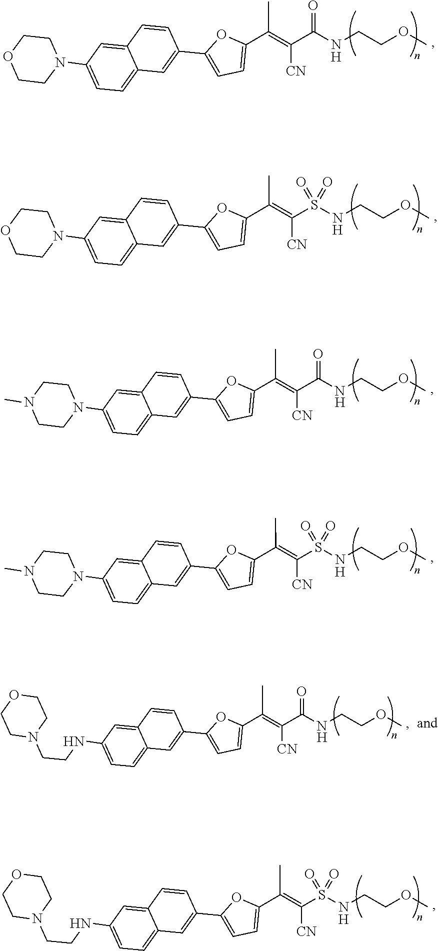

wherein n is an integer of value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

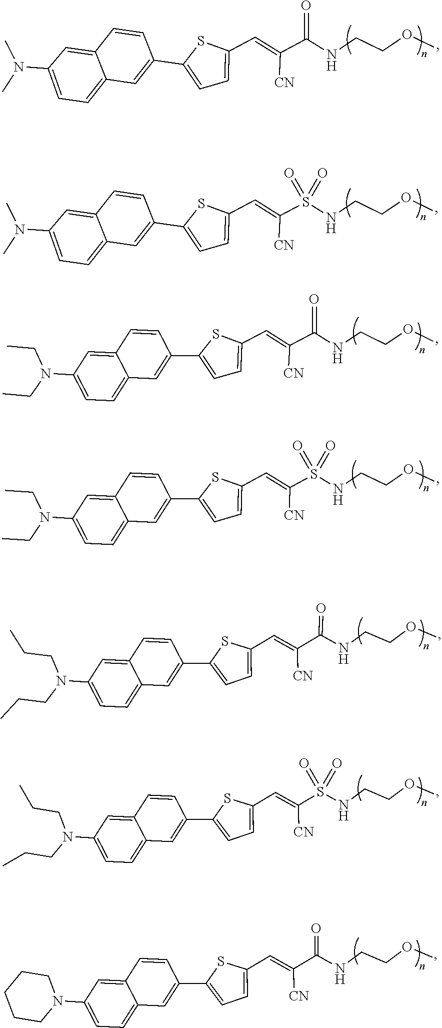

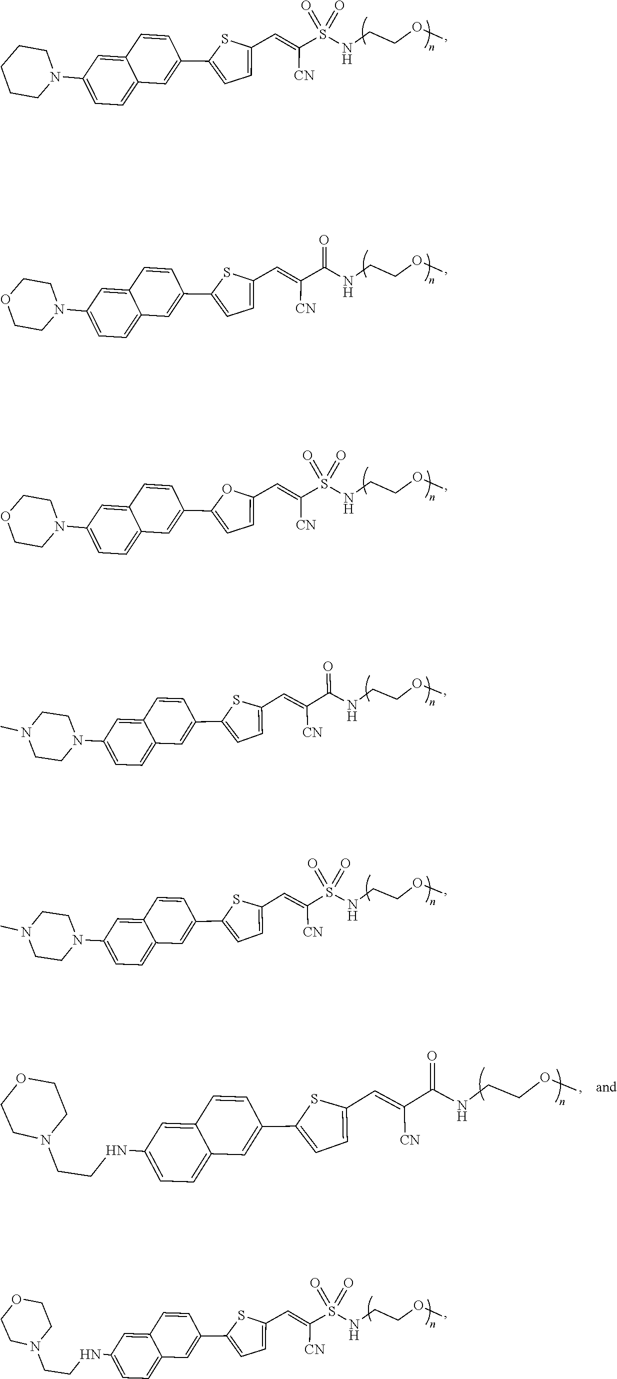

wherein n is an integer or value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

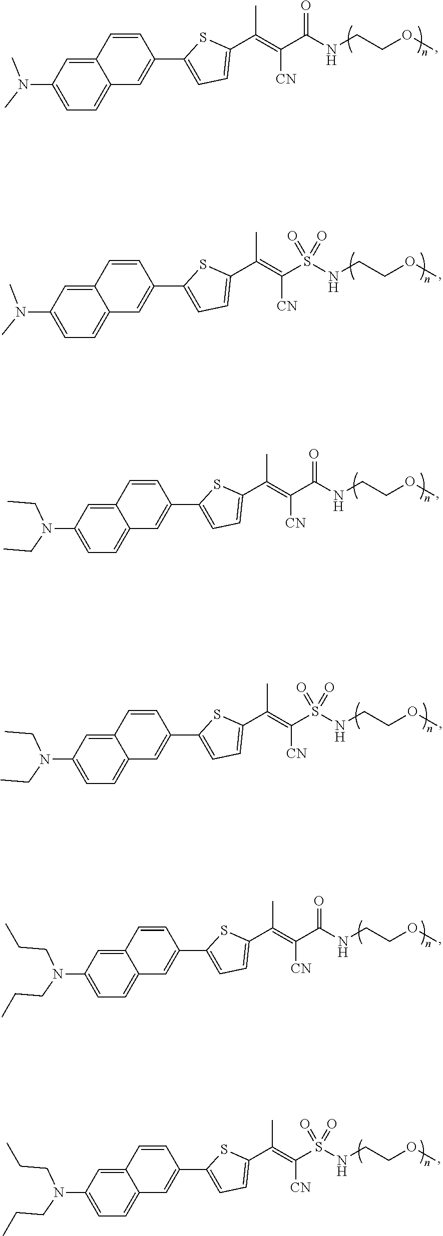

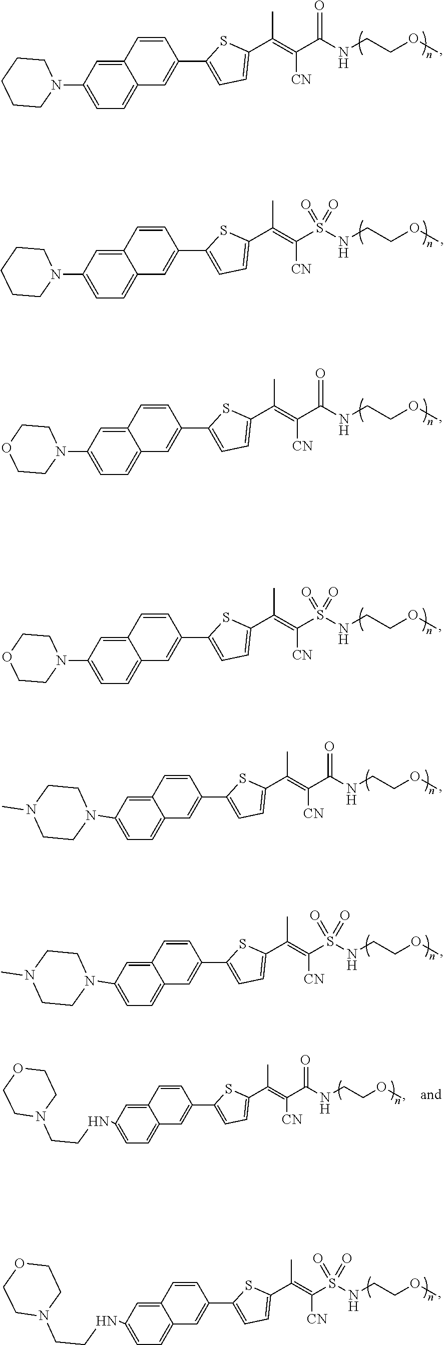

wherein n is an integer of value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

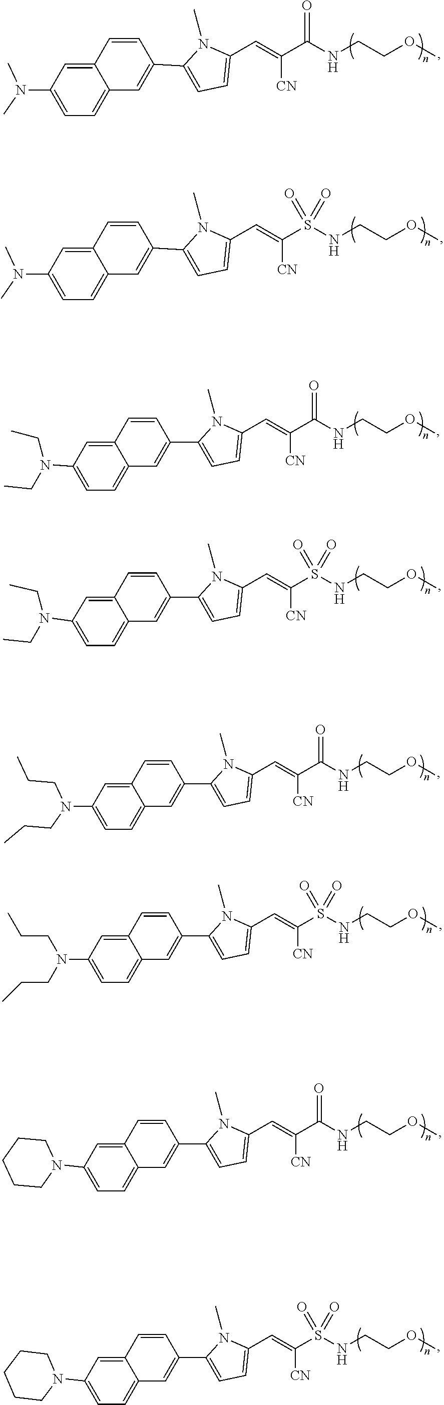

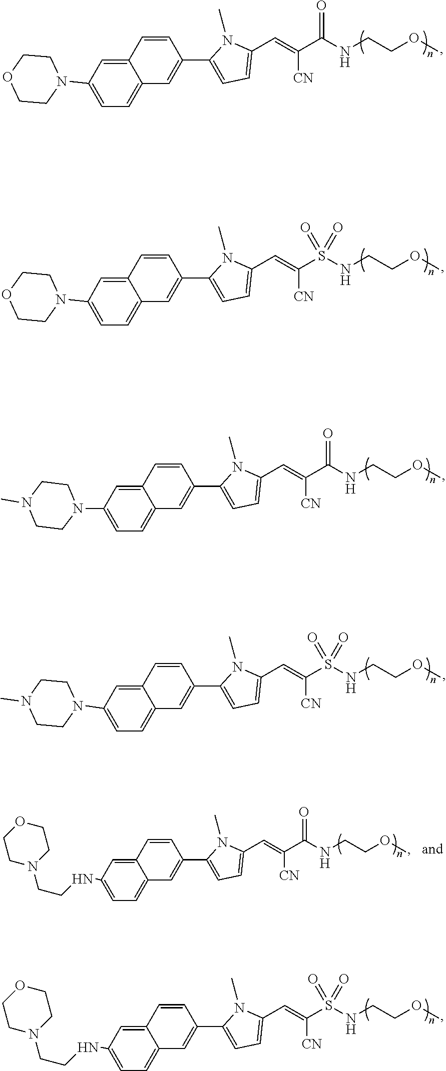

wherein n is an integer of value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

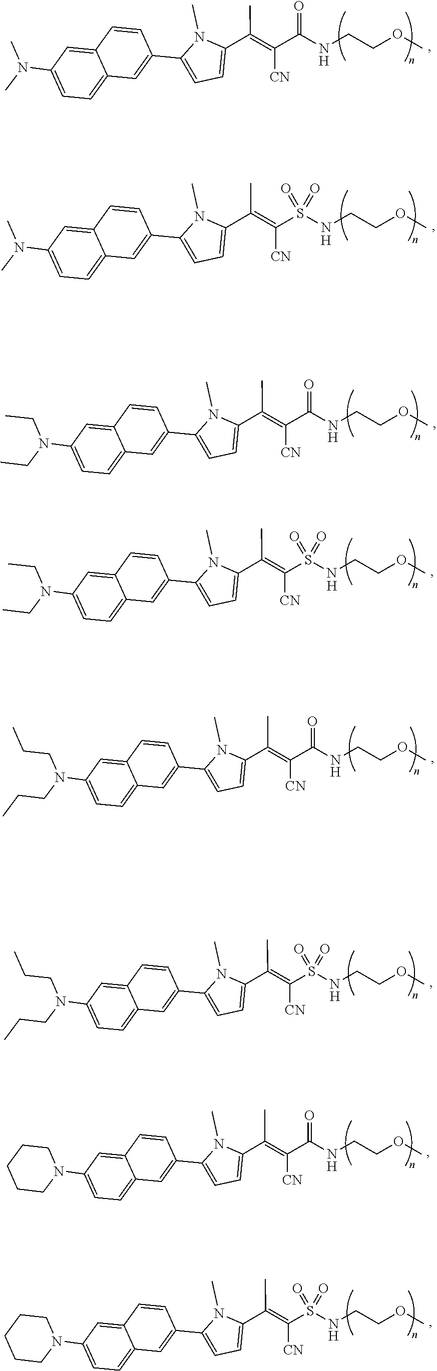

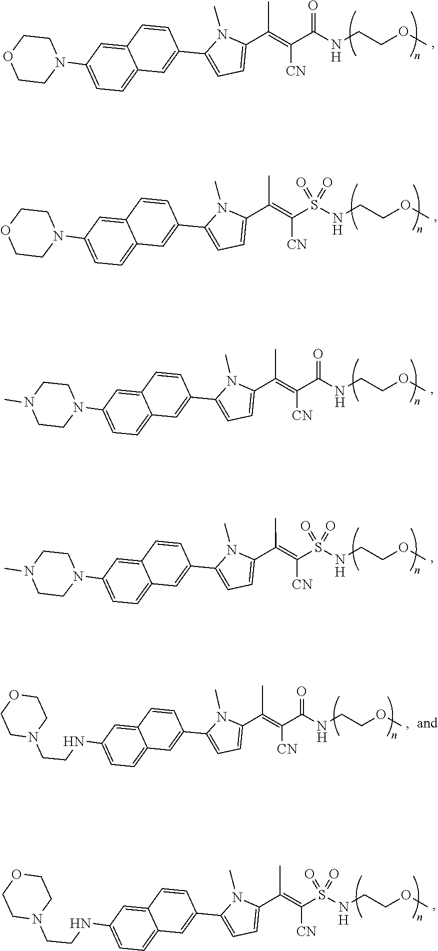

wherein n is an integer of value 1-50. In some embodiments, n is a integer of value 1-10, e.g. n is 1, 2, 3, 4, 5, 6, 7, 8, 9, or 10. In some embodiments, n is a integer of value 2-10, e.g. n is 2, 3, 4, 5, 6, 7, 8, 9, or 10.

In some embodiments, the compound is selected from a group consisting of

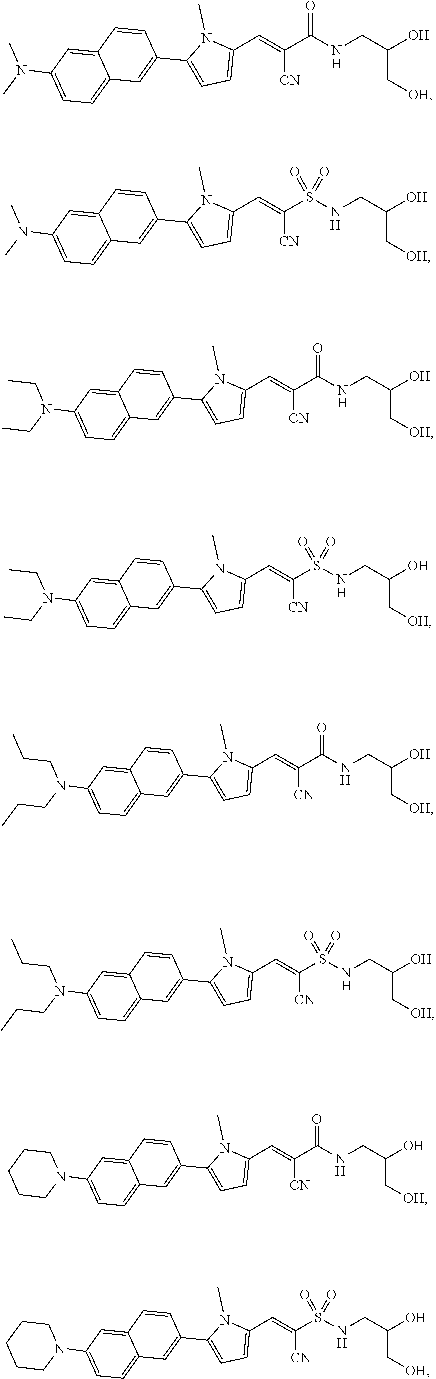

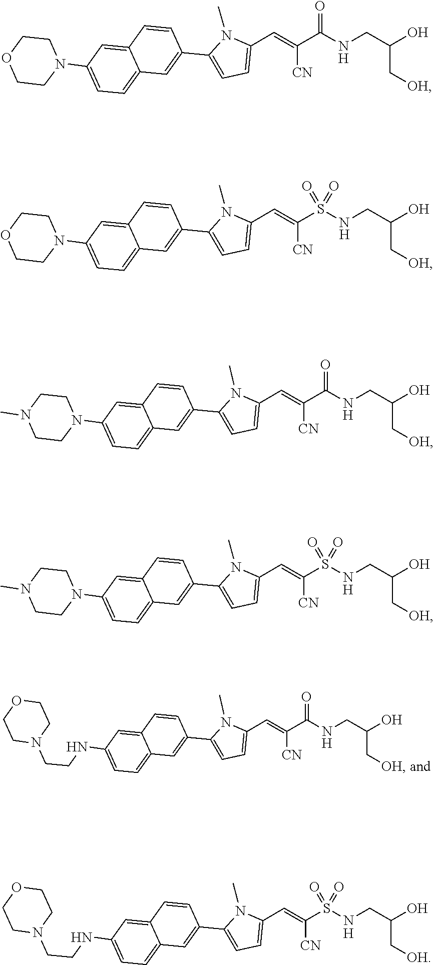

In some embodiments, the compound is selected from a group consisting of

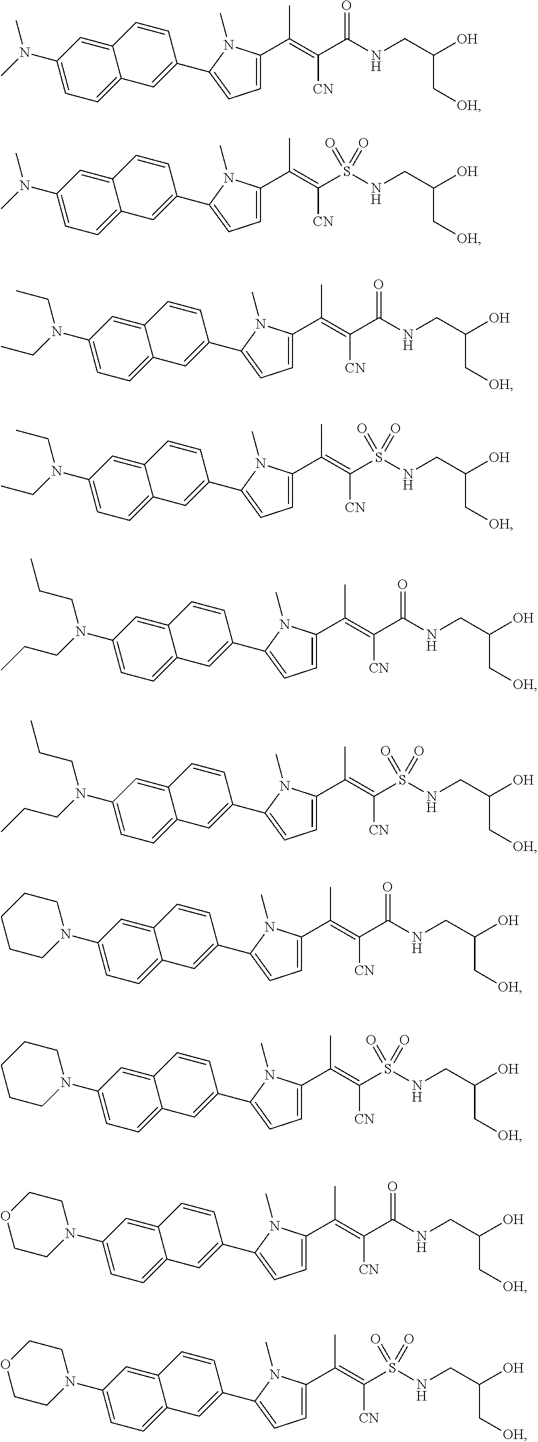

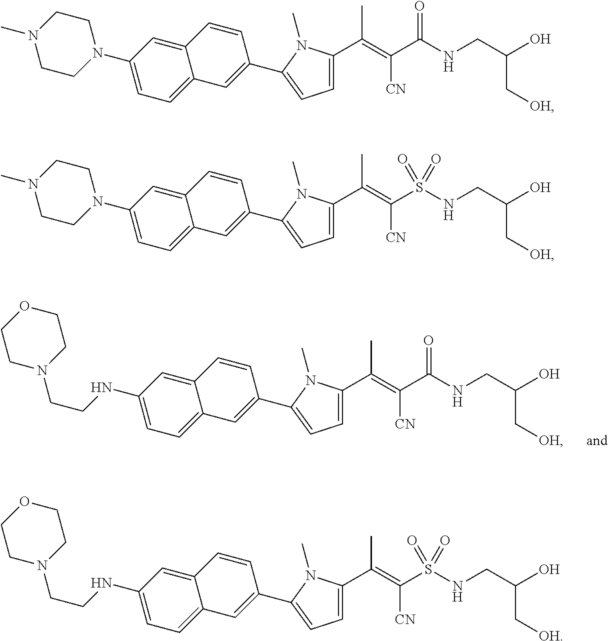 [SMILES: C/C(=C(/C#N)C(=O)NCC(O)CO)c1ccc(-c2ccc3cc(N4CCN(C)CC4)ccc3c2)n1C ; C/C(=C(/C#N)S(=O)(=O)NCC(O)CO)c1ccc(-c2ccc3cc(N4CCN(C)CC4)ccc3c2)n1C ; C/C(=C(/C#N)C(=O)NCC(O)CO)c1ccc(-c2ccc3cc(NCCN4CCOCC4)ccc3c2)n1C ; C/C(=C(/C#N)S(=O)(=O)NCC(O)CO)c1ccc(-c2ccc3cc(NCCN4CCOCC4)ccc3c2)n1C]

In some embodiments, the compound is selected from a group consisting of

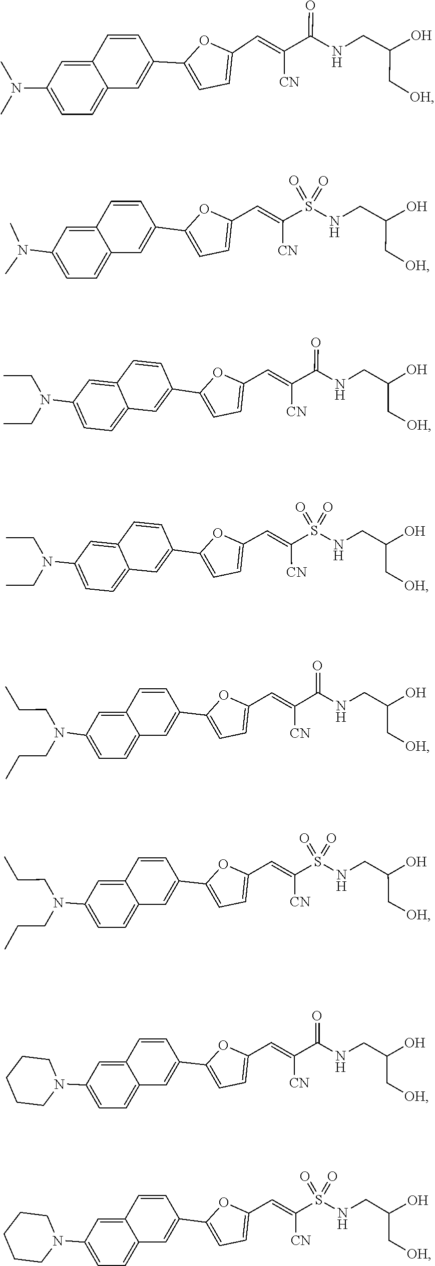

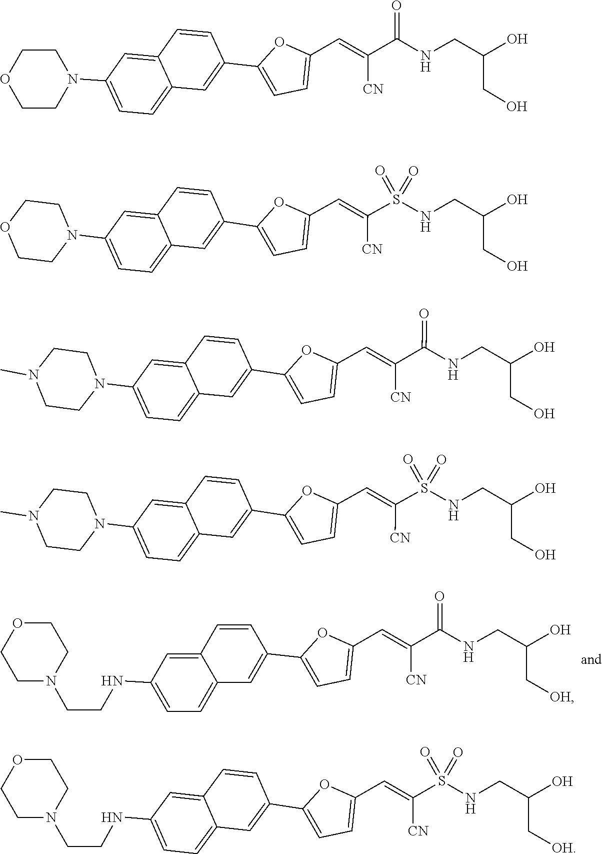 [SMILES: N#C/C(=C\c1ccc(-c2ccc3cc(N4CCOCC4)ccc3c2)o1)C(=O)NCC(O)CO ; N#C/C(=C\c1ccc(-c2ccc3cc(N4CCOCC4)ccc3c2)o1)S(=O)(=O)NCC(O)CO ; CN1CCN(c2ccc3cc(-c4ccc(/C=C(\C#N)C(=O)NCC(O)CO)o4)ccc3c2)CC1 ; CN1CCN(c2ccc3cc(-c4ccc(/C=C(\C#N)S(=O)(=O)NCC(O)CO)o4)ccc3c2)CC1 ; N#C/C(=C\c1ccc(-c2ccc3cc(NCCN4CCOCC4)ccc3c2)o1)C(=O)NCC(O)CO ; N#C/C(=C\c1ccc(-c2ccc3cc(NCCN4CCOCC4)ccc3c2)o1)S(=O)(=O)NCC(O)CO]

In some embodiments, the compound is selected from a group consisting of

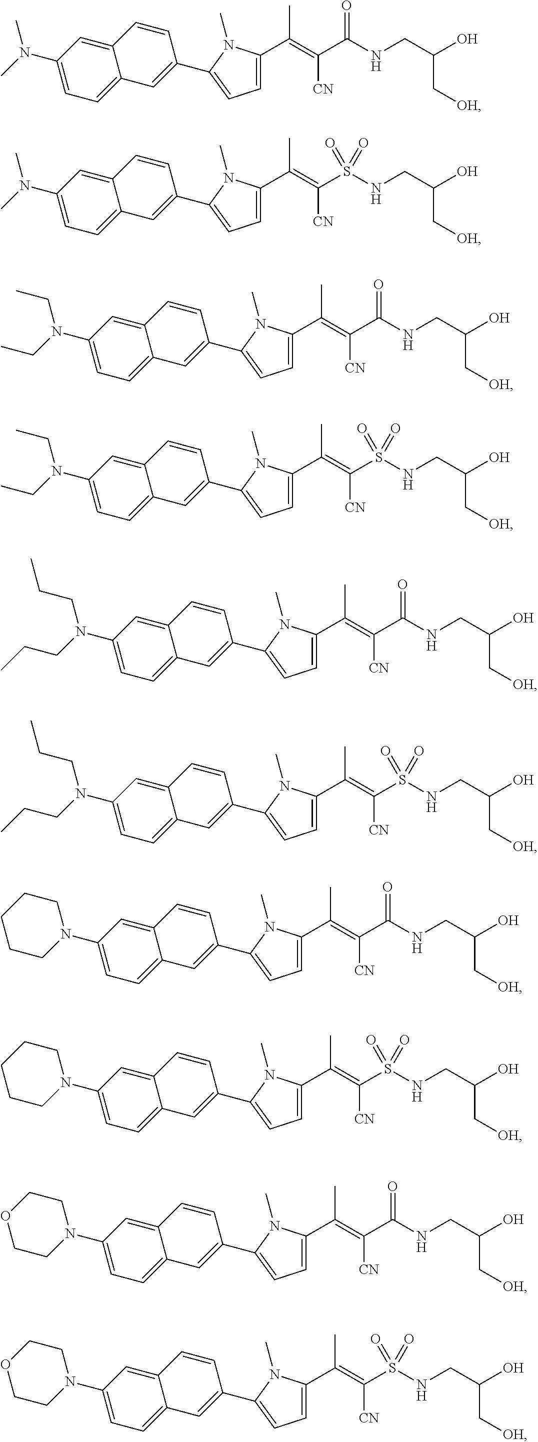

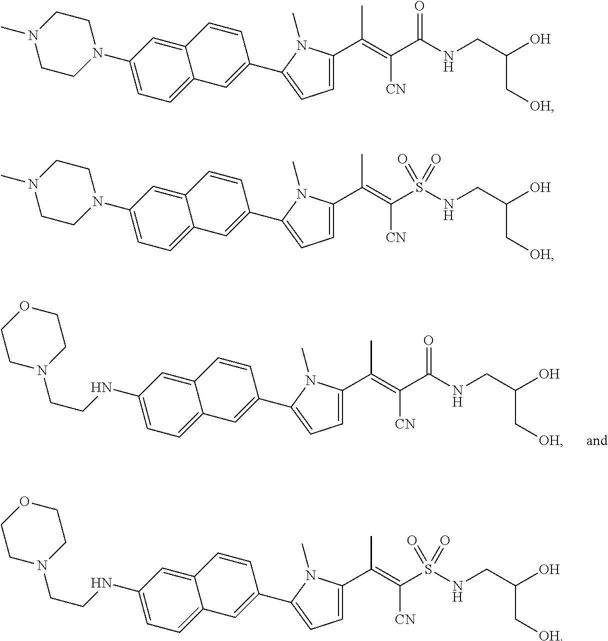 [SMILES: C/C(=C(/C#N)C(=O)NCC(O)CO)c1ccc(-c2ccc3cc(N4CCN(C)CC4)ccc3c2)n1C ; C/C(=C(/C#N)S(=O)(=O)NCC(O)CO)c1ccc(-c2ccc3cc(N4CCN(C)CC4)ccc3c2)n1C ; C/C(=C(/C#N)C(=O)NCC(O)CO)c1ccc(-c2ccc3cc(NCCN4CCOCC4)ccc3c2)n1C ; C/C(=C(/C#N)S(=O)(=O)NCC(O)CO)c1ccc(-c2ccc3cc(NCCN4CCOCC4)ccc3c2)n1C]

In some embodiments, the compound is selected from a group consisting of

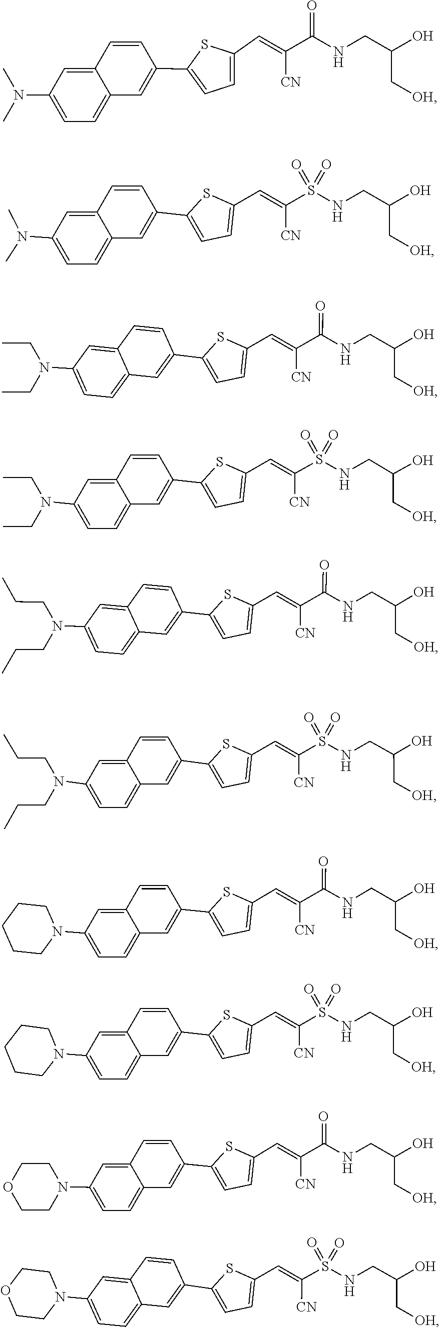

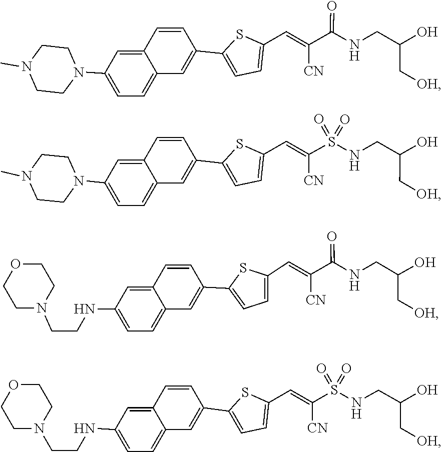 [SMILES: CN1CCN(c2ccc3cc(-c4ccc(/C=C(\C#N)C(=O)NCC(O)CO)s4)ccc3c2)CC1 ; CN1CCN(c2ccc3cc(-c4ccc(/C=C(\C#N)S(=O)(=O)NCC(O)CO)s4)ccc3c2)CC1 ; N#C/C(=C\c1ccc(-c2ccc3cc(NCCN4CCOCC4)ccc3c2)s1)C(=O)NCC(O)CO ; N#C/C(=C\c1ccc(-c2ccc3cc(NCCN4CCOCC4)ccc3c2)s1)S(=O)(=O)NCC(O)CO]

In some embodiments, the compound is selected from a group consisting of

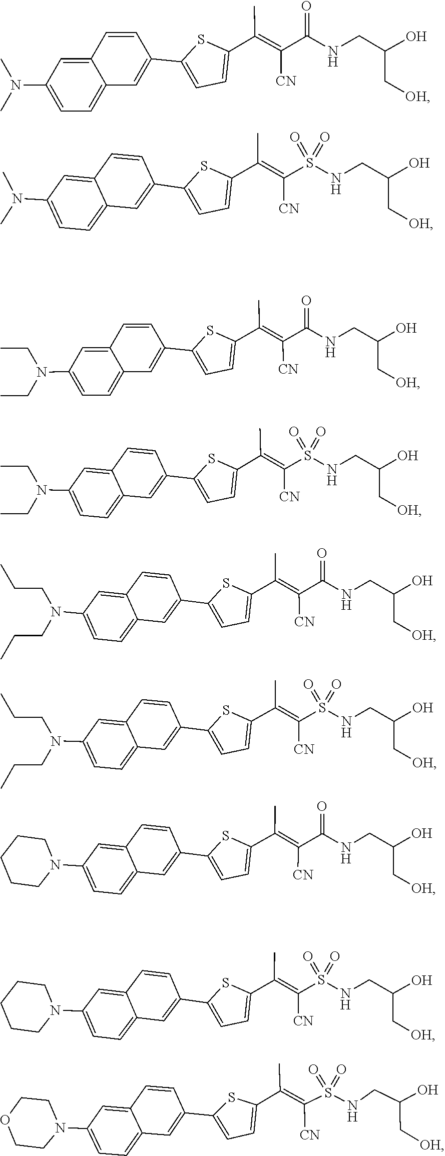

In some embodiments, the compound is selected from a group consisting of

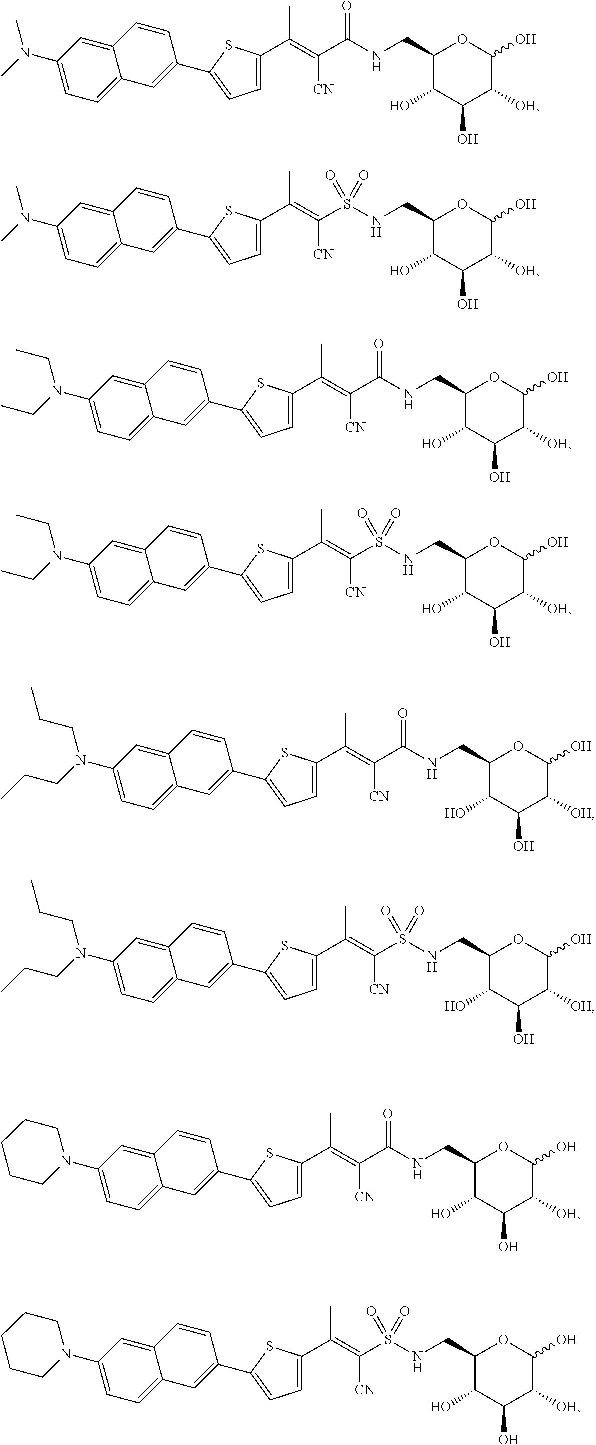

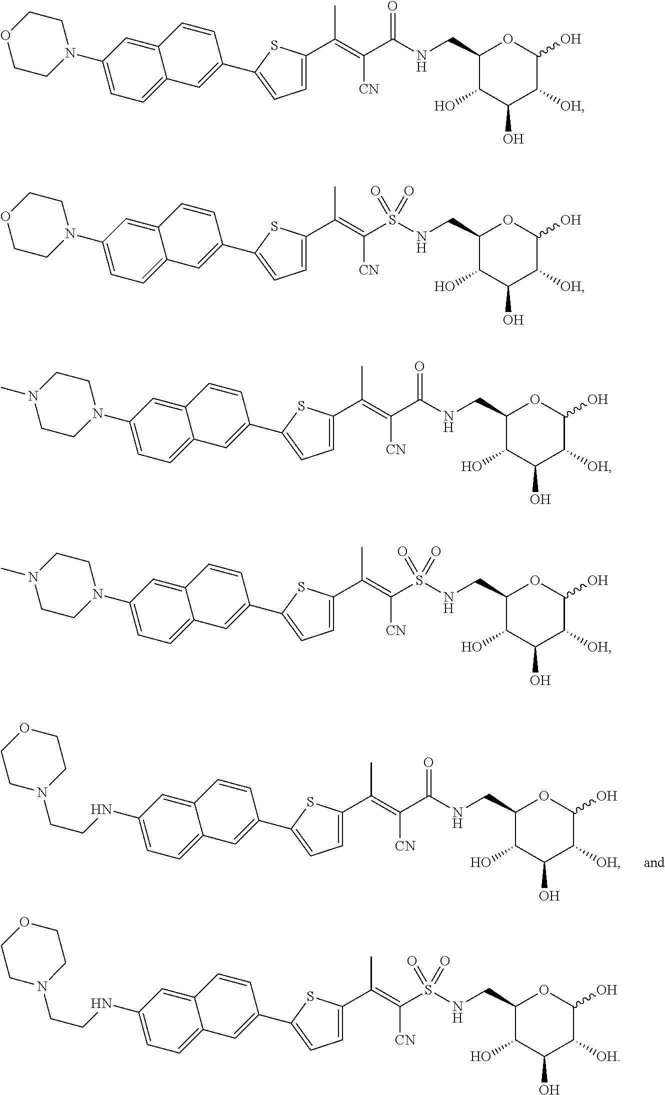

In some embodiments, the compound is selected from a group consisting of

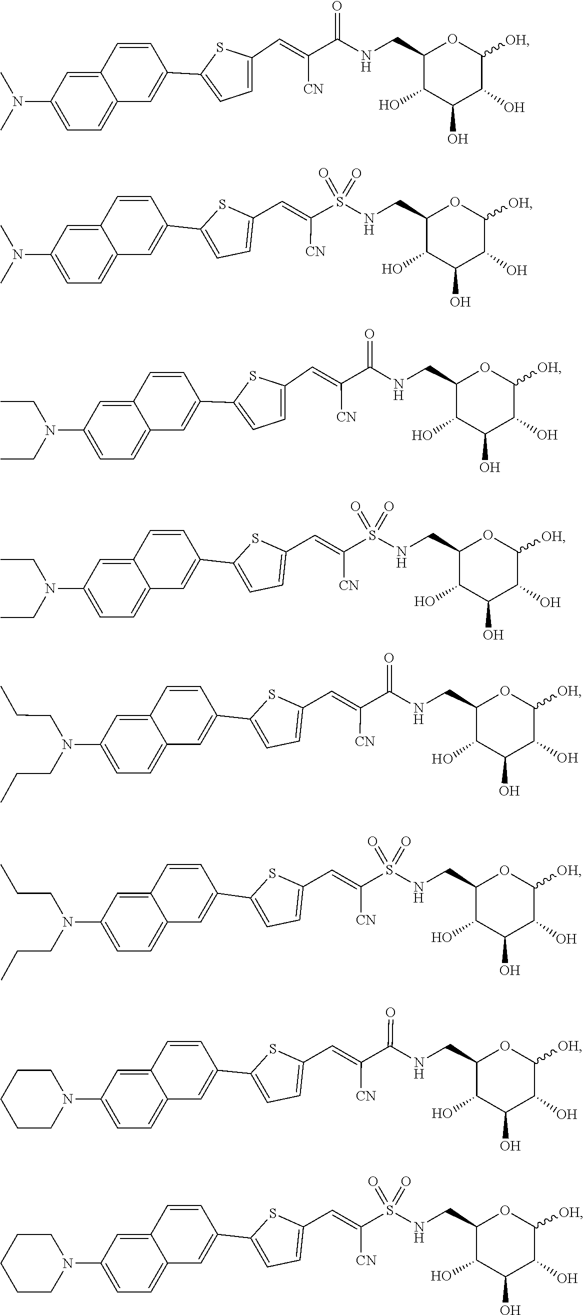

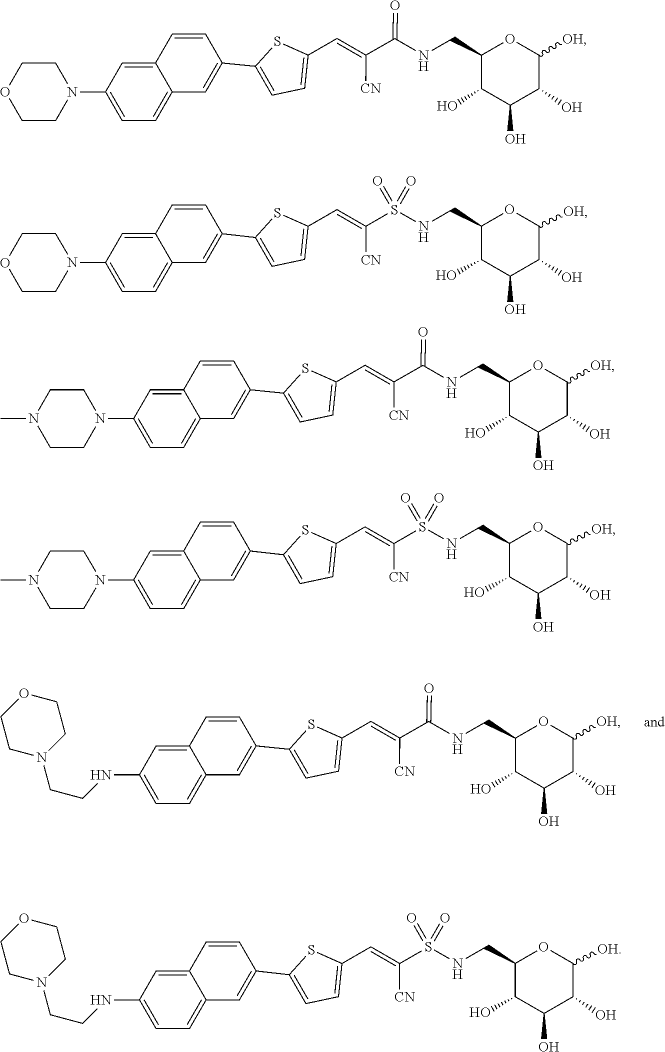

In some embodiments, the compound is selected from a group consisting of

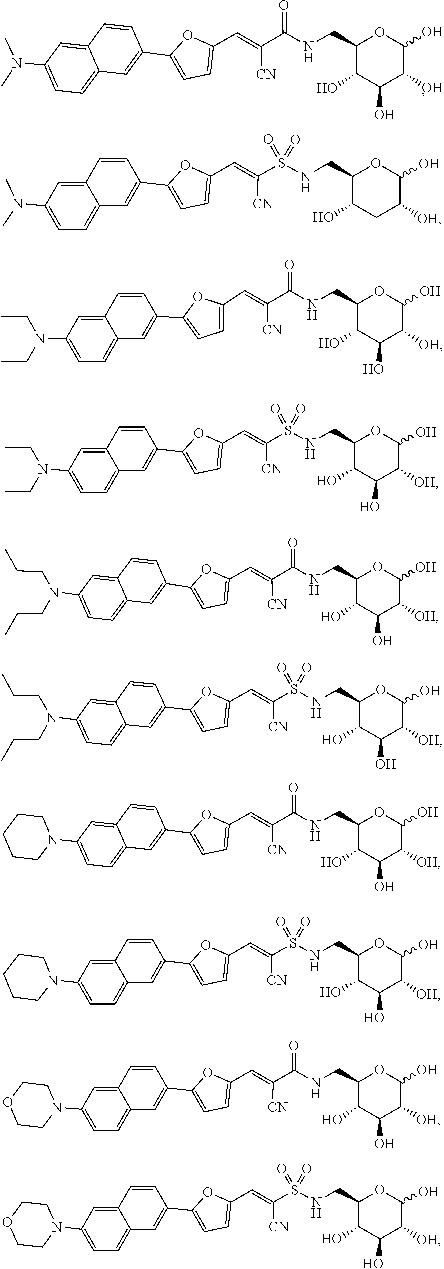

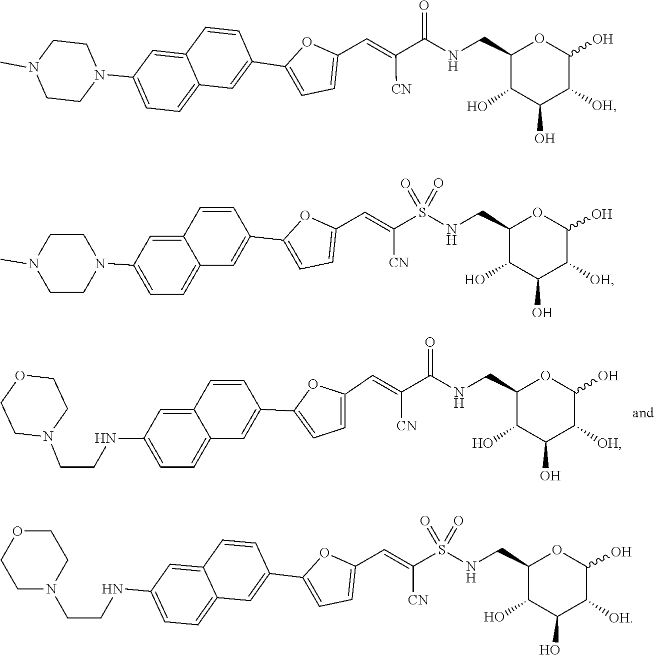 [SMILES: CN1CCN(c2ccc3cc(-c4ccc(/C=C(\C#N)C(=O)NC[C@H]5OC(O)[C@H](O)[C@@H](O)[C@@H]5O)o4)ccc3c2)CC1 ; CN1CCN(c2ccc3cc(-c4ccc(/C=C(\C#N)S(=O)(=O)NC[C@H]5OC(O)[C@H](O)[C@@H](O)[C@@H]5O)o4)ccc3c2)CC1 ; N#C/C(=C\c1ccc(-c2ccc3cc(NCCN4CCOCC4)ccc3c2)o1)C(=O)NC[C@H]1OC(O)[C@H](O)[C@@H](O)[C@@H]1O ; N#C/C(=C\c1ccc(-c2ccc3cc(NCCN4CCOCC4)ccc3c2)o1)S(=O)(=O)NC[C@H]1OC(O)[C@H](O)[C@@H](O)[C@@H]1O]

In some embodiments, the compound is selected from a group consisting of

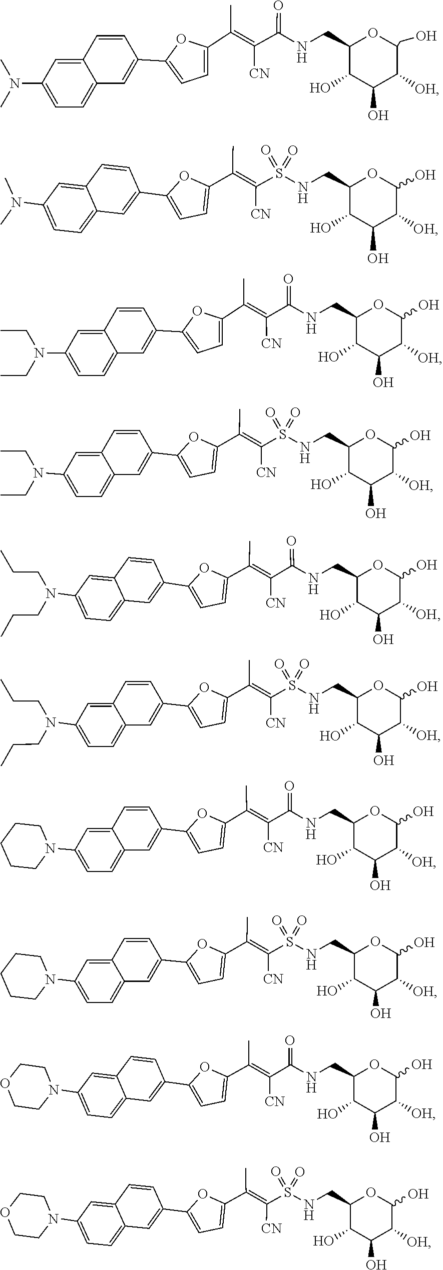

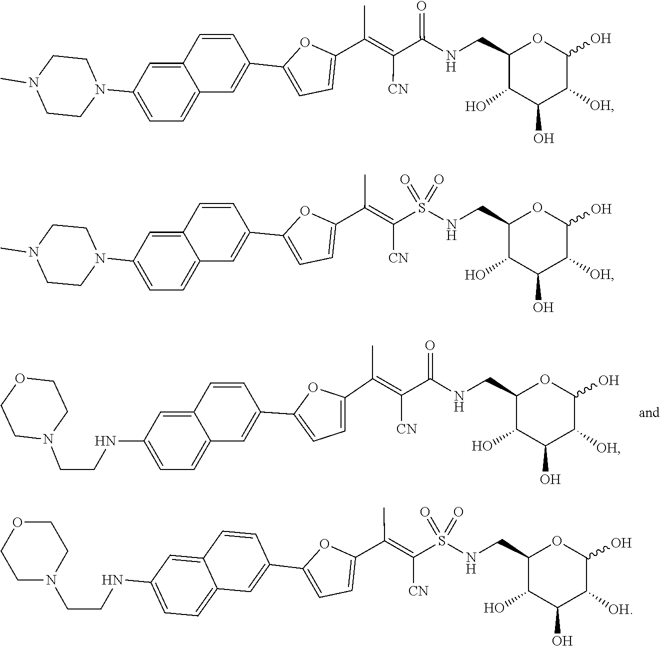 [SMILES: C/C(=C(/C#N)C(=O)NC[C@H]1OC(O)[C@H](O)[C@@H](O)[C@@H]1O)c1ccc(-c2ccc3cc(N4CCN(C)CC4)ccc3c2)o1 ; C/C(=C(/C#N)S(=O)(=O)NC[C@H]1OC(O)[C@H](O)[C@@H](O)[C@@H]1O)c1ccc(-c2ccc3cc(N4CCN(C)CC4)ccc3c2)o1 ; C/C(=C(/C#N)C(=O)NC[C@H]1OC(O)[C@H](O)[C@@H](O)[C@@H]1O)c1ccc(-c2ccc3cc(NCCN4CCOCC4)ccc3c2)o1 ; C/C(=C(/C#N)S(=O)(=O)NC[C@H]1OC(O)[C@H](O)[C@@H](O)[C@@H]1O)c1ccc(-c2ccc3cc(NCCN4CCOCC4)ccc3c2)o1]

In some embodiments, the compound is selected from a group consisting of

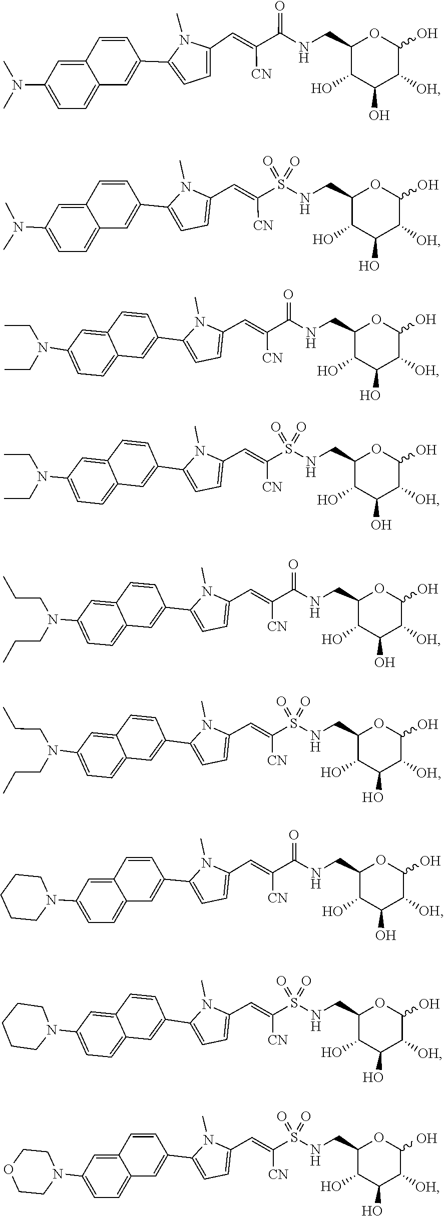

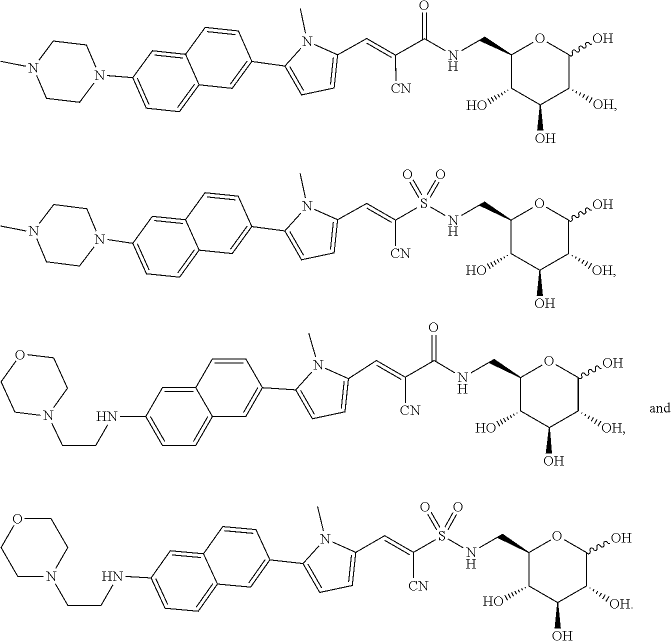 [SMILES: CN1CCN(c2ccc3cc(-c4ccc(/C=C(\C#N)C(=O)NC[C@H]5OC(O)[C@H](O)[C@@H](O)[C@@H]5O)n4C)ccc3c2)CC1 ; CN1CCN(c2ccc3cc(-c4ccc(/C=C(\C#N)S(=O)(=O)NC[C@H]5OC(O)[C@H](O)[C@@H](O)[C@@H]5O)n4C)ccc3c2)CC1 ; Cn1c(/C=C(\C#N)C(=O)NC[C@H]2OC(O)[C@H](O)[C@@H](O)[C@@H]2O)ccc1-c1ccc2cc(NCCN3CCOCC3)ccc2c1 ; Cn1c(/C=C(\C#N)S(=O)(=O)NC[C@H]2OC(O)[C@H](O)[C@@H](O)[C@@H]2O)ccc1-c1ccc2cc(NCCN3CCOCC3)ccc2c1]

In some embodiments, the compound is selected from a group consisting of

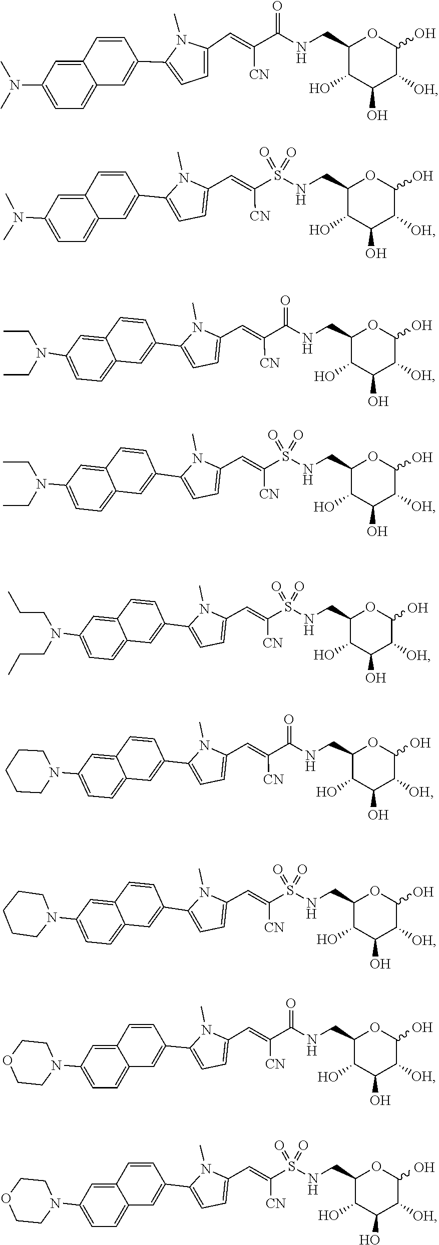

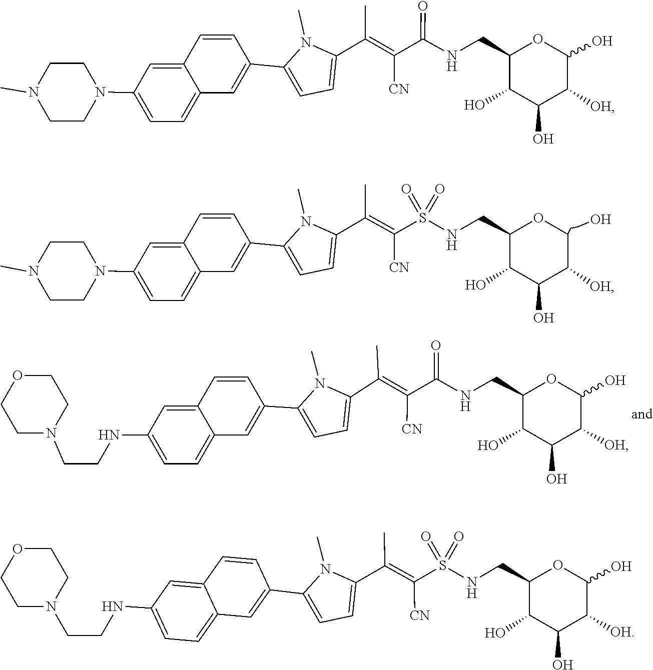 [SMILES: C/C(=C(/C#N)C(=O)NC[C@H]1OC(O)[C@H](O)[C@@H](O)[C@@H]1O)c1ccc(-c2ccc3cc(N4CCN(C)CC4)ccc3c2)n1C ; C/C(=C(/C#N)S(=O)(=O)NC[C@H]1OC(CCO)[C@H](O)[C@@H](O)[C@@H]1O)c1ccc(-c2ccc3cc(N4CCN(C)CC4)ccc3c2)n1C ; C/C(=C(/C#N)C(=O)NC[C@H]1OC(O)[C@H](O)[C@@H](O)[C@@H]1O)c1ccc(-c2ccc3cc(NCCN4CCOCC4)ccc3c2)n1C ; C/C(=C(/C#N)S(=O)(=O)NC[C@H]1OC(O)[C@H](O)[C@@H](O)[C@@H]1O)c1ccc(-c2ccc3cc(NCCN4CCOCC4)ccc3c2)n1C]

In some embodiments, the compound is selected from a group consisting of

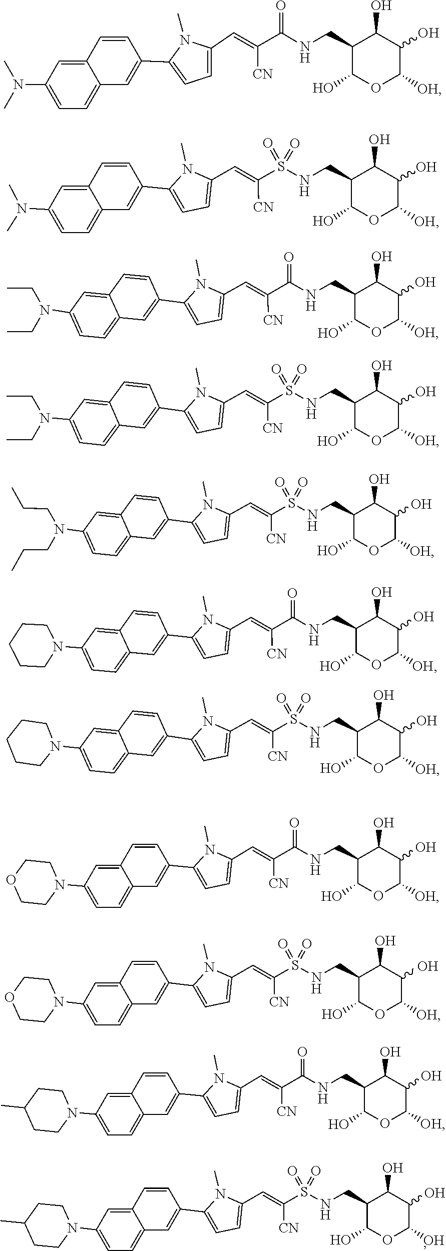

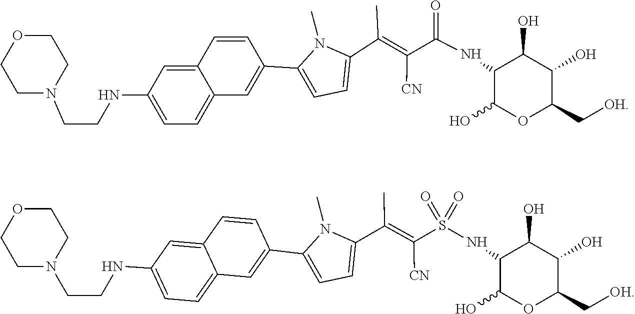

In some embodiments, the compound is selected from a group consisting of

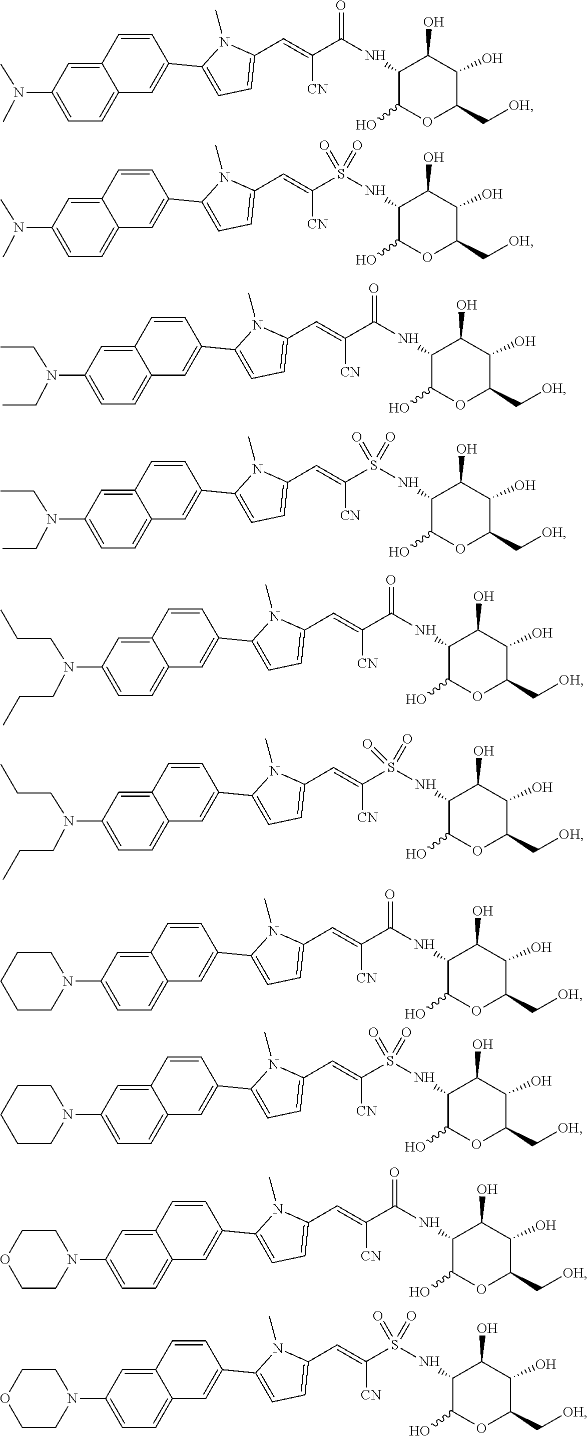

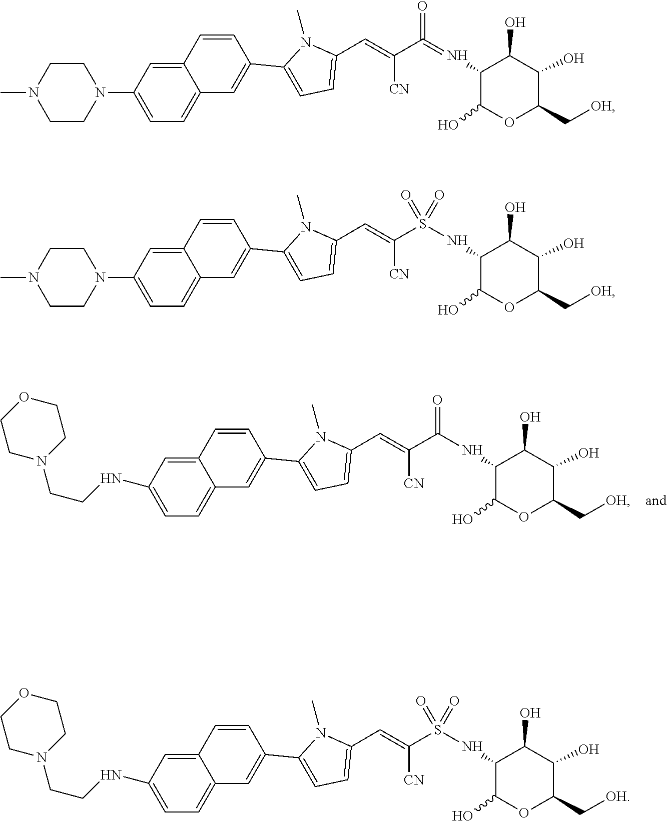

In some embodiments, the compound is selected from a group consisting of

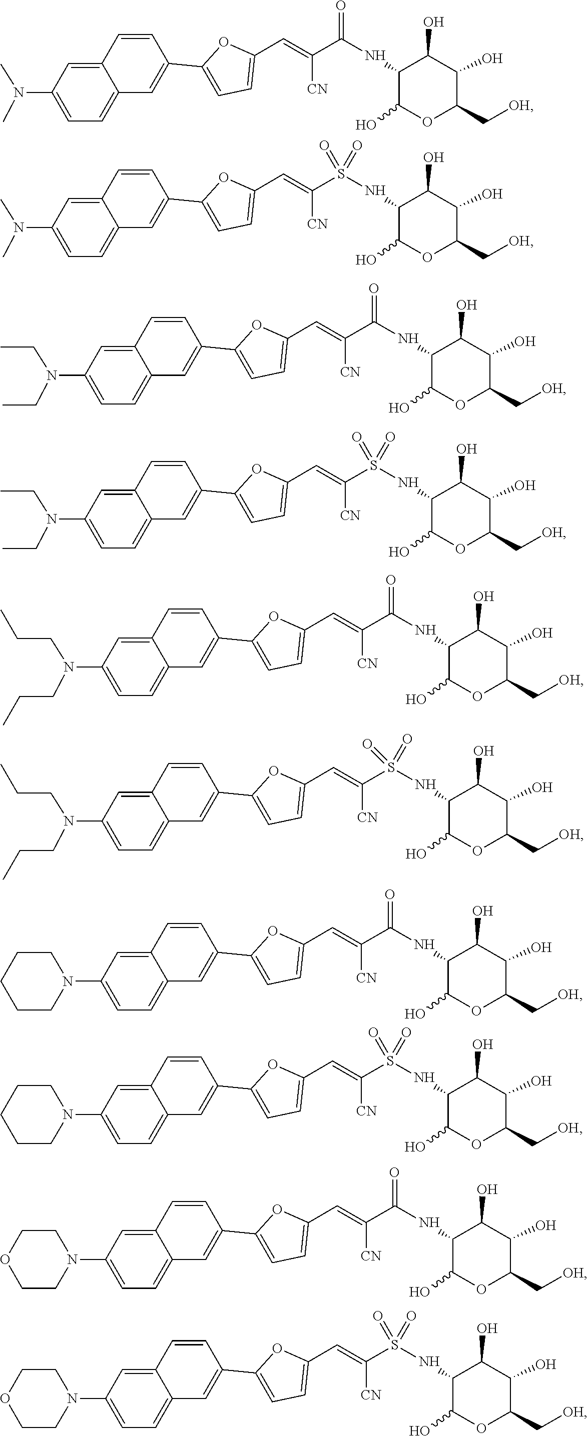

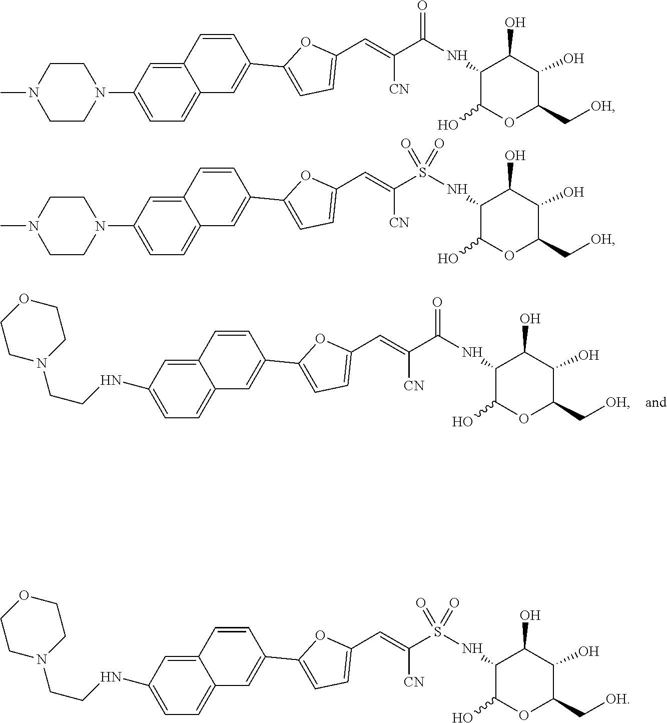

In some embodiments, the compound is selected from a group consisting of

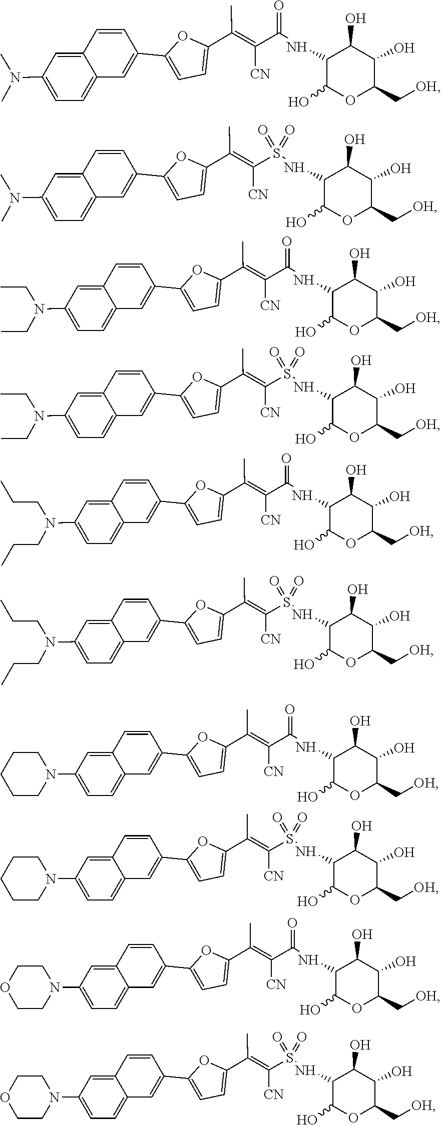

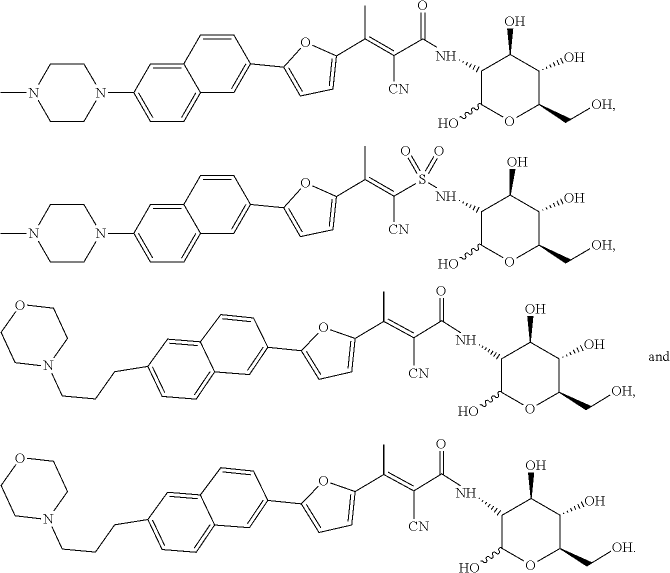

In some embodiments, the compound is selected from a group consisting of

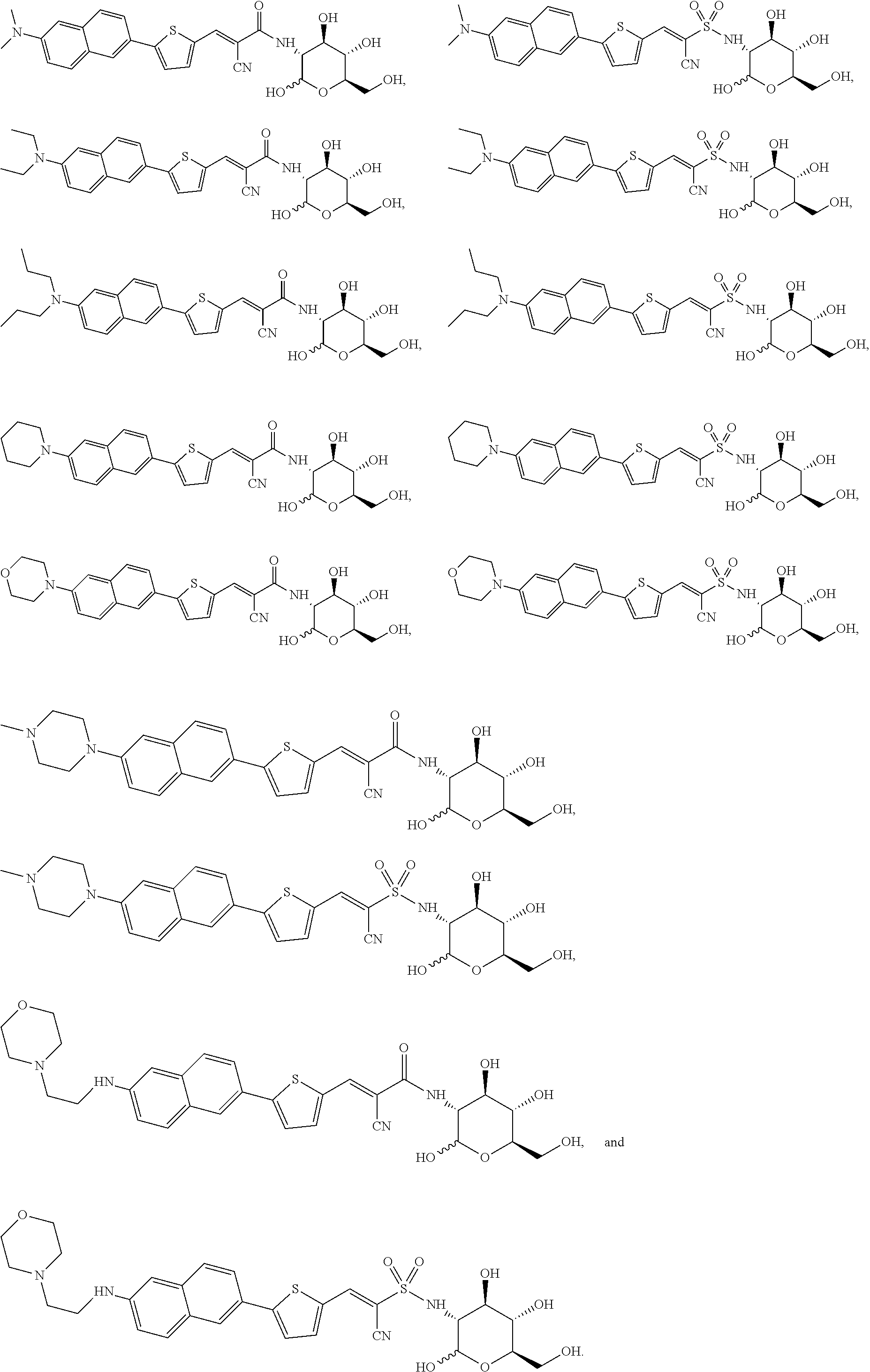

In some embodiments, the compound is selected from a group consisting of

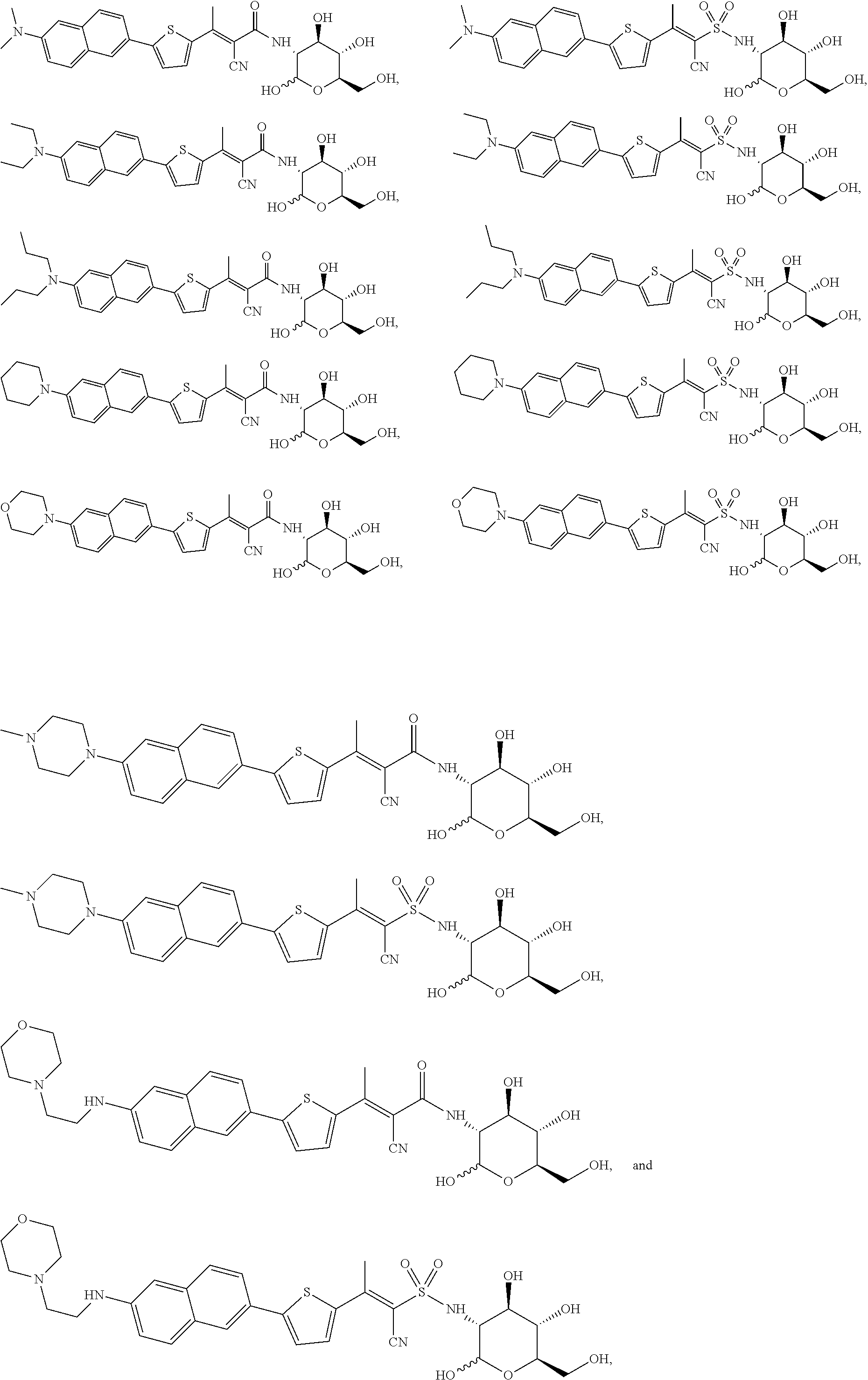

In some embodiments, the compound is:

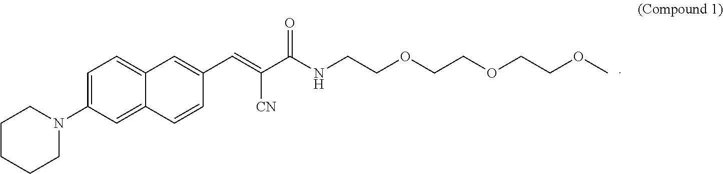 [SMILES: COCCOCCOCCNC(=O)/C(C#N)=C/c1ccc2cc(N3CCCCC3)ccc2c1]

In some embodiments, the compound is (E)-2-cyano-N-(2-(2-(2-methoxyethoxy)ethoxy)ethyl)-3-(6-(piperidin-1-yl)naphthalen-2-yl)acrylamide. In some embodiments, the compound is (Z)-2-cyano-N-(2-(2-(2-methoxyethoxy)ethoxy)ethyl)-3-(6-(piperidin-1-yl)naphthalen-2-yl)acrylamide.

In some embodiments, the compound is:

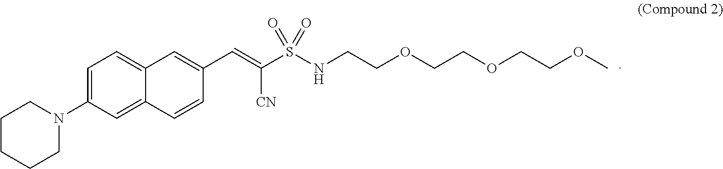 [SMILES: COCCOCCOCCNS(=O)(=O)/C(C#N)=C/c1ccc2cc(N3CCCCC3)ccc2c1]

In some embodiments, the compound is (E)-1-cyano-N-(2-(2-(2-methoxyethoxy)ethoxy)ethyl)-2-(6-(piperidin-1-yl)naphthalen-2-yl)ethenesulfonamide. In some embodiments, the compound is (Z)-1-cyano-N-(2-(2-(2-methoxyethoxy)ethoxy)ethyl)-2-(6-(piperidin-1-yl)naphthalen-2-yl)ethenesulfonamide.

In some embodiments, the compound is:

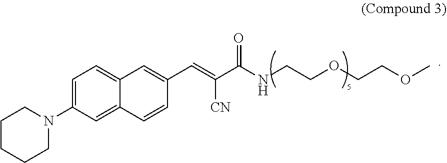

In some embodiments, the compound is (E)-2-cyano-N-(2,5,8,11,14,17-hexaoxanonadecan-19-yl)-3-(6-(piperidin-1-yl)naphthalen-2-yl)acrylamide. In some embodiments, the compound is (Z)-2-cyano-N-(2,5,8,11,14,17-hexaoxanonadecan-19-yl)-3-(6-(piperidin-1-yl)naphthalen-2-yl)acrylamide.

In some embodiments, the compound is:

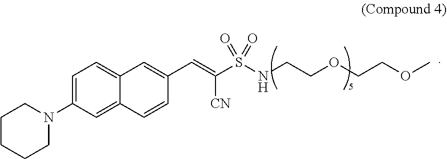

In some embodiments, the compound is (E)-1-cyano-N-(2,5,8,11,14,17-hexaoxanonadecan-19-yl)-2-(6-(piperidin-1-yl)naphthalen-2-yl)ethenesulfonamide. In some embodiments, the compound is (Z)-1-cyano-N-(2,5,8,11,14,17-hexaoxanonadecan-19-yl)-2-(6-(piperidin-1-yl)naphthalen-2-yl)ethenesulfonamide.

In some embodiments, the compound is:

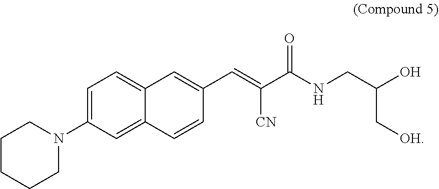 [SMILES: N#C/C(=C\c1ccc2cc(N3CCCCC3)ccc2c1)C(=O)NCC(O)CO]

In some embodiments, the compound is (E)-2-cyano-N-(2,3-dihydroxypropyl)-3-(6-(piperidin-1-yl)naphthalen-2-yl)acrylamide. In some embodiments, the compound is (Z)-2-cyano-N-(2,3-dihydroxypropyl)-3-(6-(piperidin-1-yl)naphthalen-2-yl)acrylamide.

In some embodiments, the compound is:

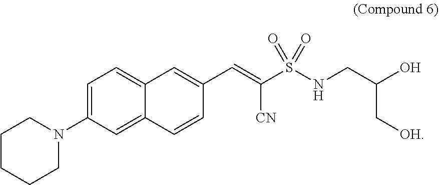 [SMILES: N#C/C(=C\c1ccc2cc(N3CCCCC3)ccc2c1)S(=O)(=O)NCC(O)CO]

In some embodiments, the compound is (E)-1-cyano-N-(2,3-dihydroxypropyl)-2-(6-(piperidin-1-yl)naphthalen-2-yl)ethenesulfonamide. In some embodiments, the compound is (Z)-1-cyano-N-(2,3-dihydroxypropyl)-2-(6-(piperidin-1-yl)naphthalen-2-yl)ethenesulfonamide.

In some embodiments, the compound is:

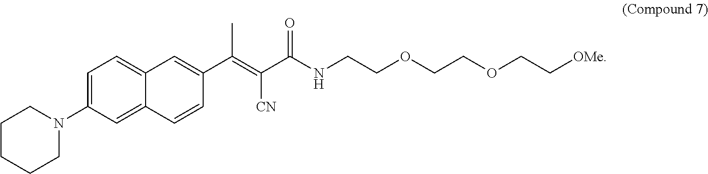 [SMILES: COCCOCCOCCNC(=O)/C(C#N)=C(\C)c1ccc2cc(N3CCCCC3)ccc2c1]

In some embodiments, the compound is (E)-2-cyano-N-(2-(2-(2-methoxyethoxy)ethoxy)ethyl)-3-(6-(piperidin-1-yl)naphthalen-2-yl)but-2-enamide. In some embodiments, the compound is (Z)-2-cyano-N-(2-(2-(2-methoxyethoxy)ethoxy)ethyl)-3-(6-(piperidin-1-yl)naphthalen-2-yl)but-2-enamide.

In some embodiments, the compound is:

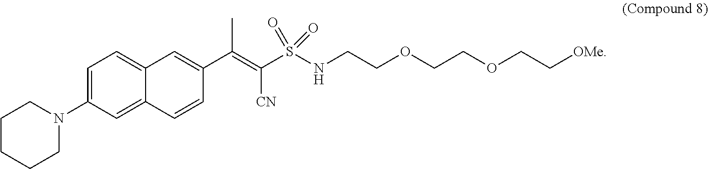 [SMILES: COCCOCCOCCNS(=O)(=O)/C(C#N)=C(\C)c1ccc2cc(N3CCCCC3)ccc2c1]

In some embodiments, the compound is (E)-1-cyano-N-(2-(2-(2-methoxyethoxy)ethoxy)ethyl)-2-(6-(piperidin-1-yl)naphthalen-2-yl)prop-1-ene-1-sulfonamide. In some embodiments, the compound is (Z)-1-cyano-N-(2-(2-(2-methoxyethoxy)ethoxy)ethyl)-2-(6-(piperidin-1-yl)naphthalen-2-yl)prop-1-ene-1-sulfonamide.

In some embodiments, the compound is:

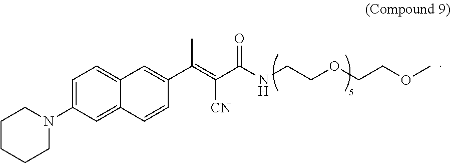

In some embodiments, the compound is (E)-2-cyano-N-(2,5,8,11,14,17-hexaoxanonadecan-19-yl)-3-(6-(piperidin-1-yl)naphthalen-2-yl)but-2-enamide. In some embodiments, the compound is (Z)-2-cyano-N-(2,5,8,11,14,17-hexaoxanonadecan-19-yl)-3-(6-(piperidin-1-yl)naphthalen-2-yl)but-2-enamide.

In some embodiments, the compound is:

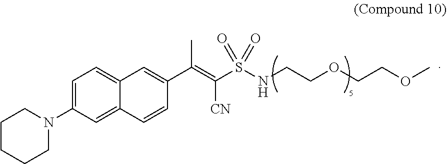

In some embodiments, the compound is (E)-1-cyano-N-(2,5,8,11,14,17-hexaoxanonadecan-19-yl)-2-(6-(piperidin-1-yl)naphthalen-2-yl)prop-1-ene-1-sulfonamide. In some embodiments, the compound is (Z)-1-cyano-N-(2,5,8,11,14,17-hexaoxanonadecan-19-yl)-2-(6-(piperidin-1-yl)naphthalen-2-yl)prop-1-ene-1-sulfonamide.

In some embodiments, the compound is:

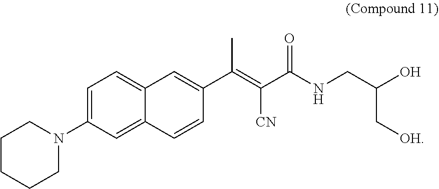 [SMILES: C/C(=C(/C#N)C(=O)NCC(O)CO)c1ccc2cc(N3CCCCC3)ccc2c1]

In some embodiments, the compound is (E)-2-cyano-N-(2,3-dihydroxypropyl)-3-(6-(piperidin-1-yl)naphthalen-2-yl)but-2-enamide. In some embodiments, the compound is (Z)-2-cyano-N-(2,3-dihydroxypropyl)-3-(6-(piperidin-1-yl)naphthalen-2-yl)but-2-enamide.

In some embodiments, the compound is:

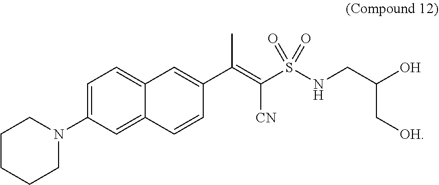 [SMILES: C/C(=C(/C#N)S(=O)(=O)NCC(O)CO)c1ccc2cc(N3CCCCC3)ccc2c1]

In some embodiments, the compound is (E)-1-cyano-N-(2,3-dihydroxypropyl)-2-(6-(piperidin-1-yl)naphthalen-2-yl)prop-1-ene-1-sulfonamide. In some embodiments, the compound is (Z)-1-cyano-N-(2,3-dihydroxypropyl)-2-(6-(piperidin-1-yl)naphthalen-2-yl)prop-1-ene-1-sulfonamide.

In some embodiments, the compound is:

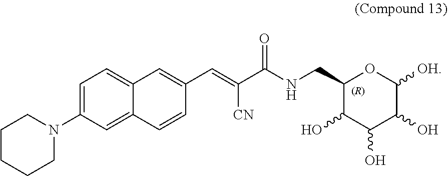

In some embodiments, the compound is (R,E)-2-cyano-3-(6-(piperidin-1-yl)naphthalen-2-yl)-N-((3,4,5,6-tetrahydroxytetrahydro-2H-pyran-2-yl)methyl)acrylamide. In some embodiments, the compound is (R,Z)-2-cyano-3-(6-(piperidin-1-yl)naphthalen-2-yl)-N-((3,4,5,6-tetrahydroxytetrahydro-2H-pyran-2-yl)methyl)acrylamide.

In some embodiments, the compound is:

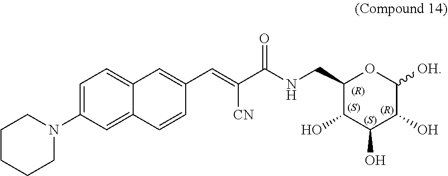

In some embodiments, the compound is (E)-2-cyano-3-(6-(piperidin-1-yl)naphthalen-2-yl)-N-(((2R,3S,4S,5R)-3,4,5,6-tetrahydroxytetrahydro-2H-pyran-2-yl)methyl)acrylamide. In some embodiments, the compound is (Z)-2-cyano-3-(6-(piperidin-1-yl)naphthalen-2-yl)-N-(((2R,3S,4S,5R)-3,4,5,6-tetrahydroxytetrahydro-2H-pyran-2-yl)methyl)acrylamide.

In some embodiments, the compound is:

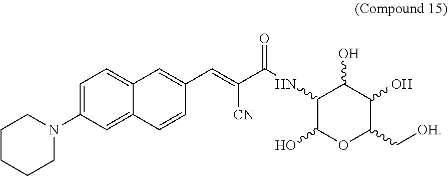 [SMILES: N#C/C(=C\c1ccc2cc(N3CCCCC3)ccc2c1)C(=O)NC1C(O)OC(CO)C(O)C1O]

In some embodiments, the compound is (E)-2-cyano-3-(6-(piperidin-1-yl)naphthalen-2-yl)-N-(2,4,5-trihydroxy-6-(hydroxymethyl)tetrahydro-2H-pyran-3-yl)acrylamide. In some embodiments, the compound is (Z)-2-cyano-3-(6-(piperidin-1-yl)naphthalen-2-yl)-N-(2,4,5-trihydroxy-6-(hydroxymethyl)tetrahydro-2H-pyran-3-yl)acrylamide.

In some embodiments, the compound is:

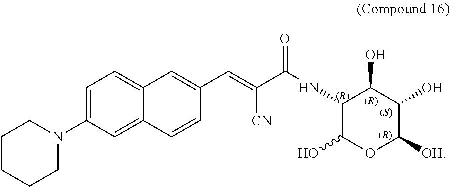

In some embodiments, the compound is (E)-2-cyano-3-(6-(piperidin-1-yl)naphthalen-2-yl)-N-((3R,4R,5S,6R)-2,4,5-trihydroxy-6-(hydroxymethyl)tetrahydro-2H-pyran-3-yl)acrylamide. In some embodiments, the compound is (Z)-2-cyano-3-(6-(piperidin-1-yl)naphthalen-2-yl)-N-((3R,4R,5S,6R)-2,4,5-trihydroxy-6-(hydroxymethyl)tetrahydro-2H-pyran-3-yl)acrylamide.

In some embodiments, the compound is:

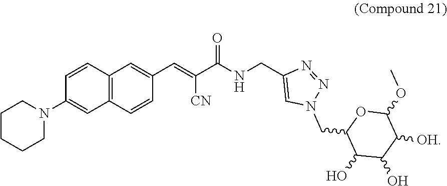 [SMILES: COC1OC(Cn2cc(CNC(=O)/C(C#N)=C/c3ccc4cc(N5CCCCC5)ccc4c3)nn2)C(O)C(O)C1O]

In some embodiments, the compound is (E)-2-cyano-3-(6-(piperidin-1-yl)naphthalen-2-yl)-N-((1-((3,4,5-trihydroxy-6-methoxytetrahydro-2H-pyran-2-yl)methyl)-1H-1,2,3-triazol-4-yl)methyl)acrylamide. In some embodiments, the compound is (Z)-2-cyano-3-(6-(piperidin-1-yl)naphthalen-2-yl)-N-((1-((3,4,5-trihydroxy-6-methoxytetrahydro-2H-pyran-2-yl)methyl)-1H-1,2,3-triazol-4-yl)methyl)acrylamide.

In some embodiments, the compound is:

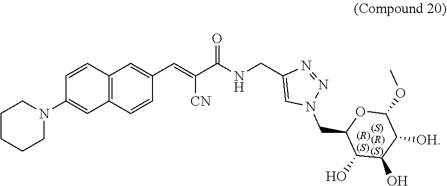

In some embodiments, the compound is (E)-2-cyano-3-(6-(piperidin-1-yl)naphthalen-2-yl)-N-((1-(((2R,3S,4S,5R,6S)-3,4,5-trihydroxy-6-methoxytetrahydro-2H-pyran-2-yl)methyl)-1H-1,2,3-triazol-4-yl)methyl)acrylamide. In some embodiments, the compound is (Z)-2-cyano-3-(6-(piperidin-1-yl)naphthalen-2-yl)-N-((1-(((2R,3S,4S,5R,6S)-3,4,5-trihydroxy-6-methoxytetrahydro-2H-pyran-2-yl)methyl)-1H-1,2,3-triazol-4-yl)methyl)acrylamide.

In some embodiments, the compound is:

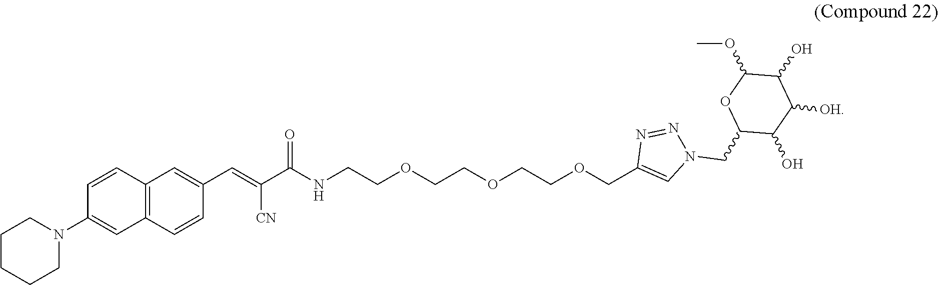 [SMILES: COC1OC(Cn2cc(COCCOCCOCCNC(=O)/C(C#N)=C/c3ccc4cc(N5CCCCC5)ccc4c3)nn2)C(O)C(O)C1O]

In some embodiments, the compound is (E)-2-cyano-3-(6-(piperidin-1-yl)naphthalen-2-yl)-N-(2-(2-(2-((1-((3,4,5-trihydroxy-6-methoxytetrahydro-2H-pyran-2-yl)methyl)-1H-1,2,3-triazol-4-yl)methoxy)ethoxy)ethoxy)ethyl)acrylamide. In some embodiments, the compound is (Z)-2-cyano-3-(6-(piperidin-1-yl)naphthalen-2-yl)-N-(2-(2-(2-((1-((3,4,5-trihydroxy-6-methoxytetrahydro-2H-pyran-2-yl)methyl)-1H-1,2,3-triazol-4-yl)methoxy)ethoxy)ethoxy)ethyl)acrylamide.

In some embodiments, the compound is:

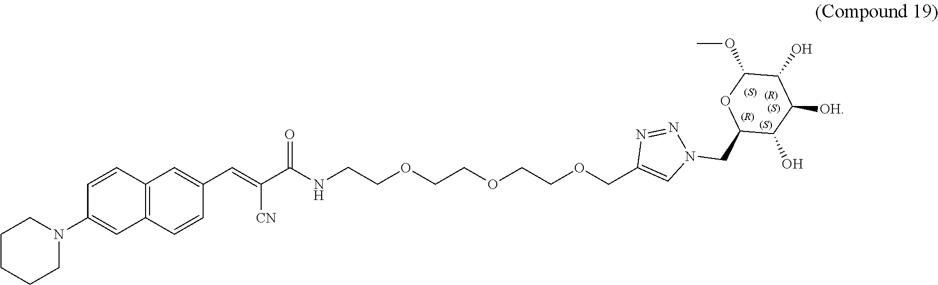

In some embodiments, the compound is (E)-2-cyano-3-(6-(piperidin-1-yl)naphthalen-2-yl)-N-(2-(2-(2-((1-(((2R,3S,4S,5R,6S)-3,4,5-trihydroxy-6-methoxytetrahydro-2H-pyran-2-yl)methyl)-1H-1,2,3-triazol-4-yl)methoxy)ethoxy)ethoxy)ethyl)acrylamide. In some embodiments, the compound is (Z)-2-cyano-3-(6-(piperidin-1-yl)naphthalen-2-yl)-N-(2-(2-(2-((1-(((2R,3S,4S,5R,6S)-3,4,5-trihydroxy-6-methoxytetrahydro-2H-pyran-2-yl)methyl)-1H-1,2,3-triazol-4-yl)methoxy)ethoxy)ethoxy)ethyl)acrylamide.

In certain embodiments, the compound is

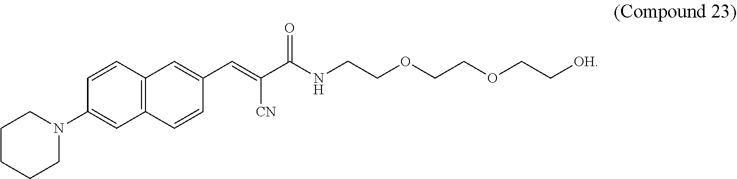 [SMILES: N#C/C(=C\c1ccc2cc(N3CCCCC3)ccc2c1)C(=O)NCCOCCOCCO]

In certain embodiments, the compound is a pharmaceutically acceptable salt or solvate of Compound 23.

In certain embodiments, the compound is

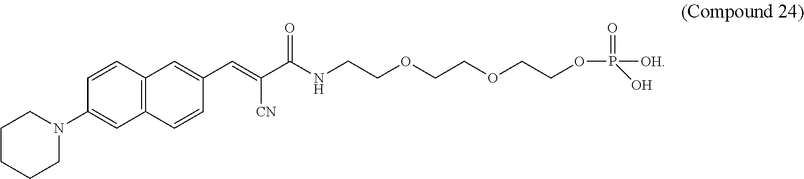 [SMILES: N#C/C(=C\c1ccc2cc(N3CCCCC3)ccc2c1)C(=O)NCCOCCOCCOP(=O)(O)O]

In certain embodiments, the compound is a pharmaceutically acceptable salt or solvate of Compound 24.

In certain embodiments, the compound is

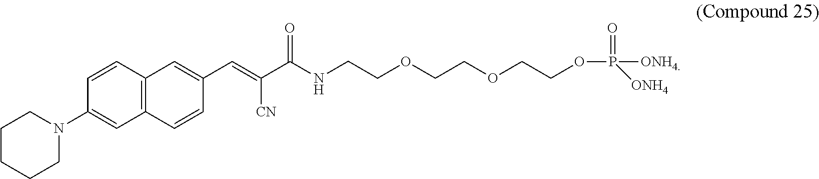

In certain embodiments, the compound is

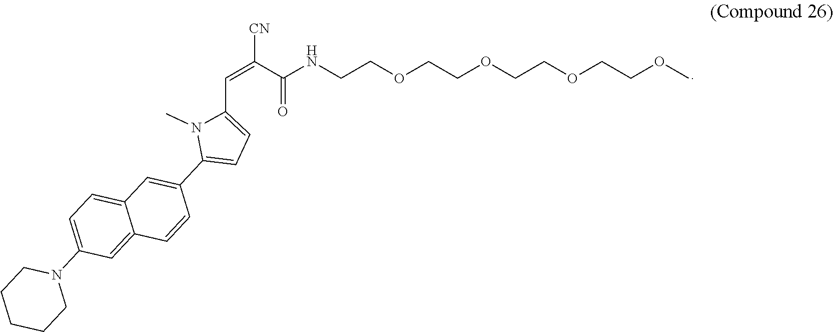 [SMILES: COCCOCCOCCOCCNC(=O)/C(C#N)=C\c1ccc(-c2ccc3cc(N4CCCCC4)ccc3c2)n1C]

In certain embodiments, the compound is

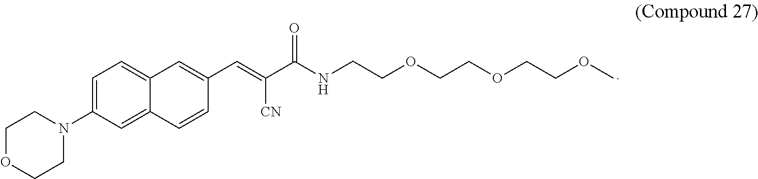 [SMILES: COCCOCCOCCNC(=O)/C(C#N)=C/c1ccc2cc(N3CCOCC3)ccc2c1]

In some embodiments, each Ar₁ is independently a substituted or unsubstituted naphthylene or a substituted or unsubstituted phenylene. In some embodiments, Ar₂ is a substituted or unsubstituted naphthylene or a substituted or unsubstituted phenylene. In some embodiments, Ar₂ is a substituted or unsubstituted pyridyl, substituted or unsubstituted pyrimidyl, substituted or unsubstituted pyrazinyl or substituted or unsubstituted pyradizinyl. In some embodiments, Ar₂ is a substituted or unsubstituted pyridyl.

In some embodiments, the substituent EDG in Formula II may be an electron donating group. In some embodiments, EDG is any electron donating group known in the art. In some embodiments, it is any atom or functional group that is capable of donating some of its electron density into a conjugated pi system via resonance or inductive electron withdrawal, thus making the pi system more nucleophilic. In some embodiments, the EDG is —OR₄₉, —NR₅₀R₅₁, —SR₅₂, —PR₅₃R₅₄, —NR₅₅C(O)R₅₆, C₁-C₁₀ alkyl, C₁-C₁₀ heteroalkyl, cycloalkyl, heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₅₇; wherein each R₅₇ is independently halogen, —OR₅₈, —NR₅₉R₆₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; each of R₄₉, R₅₀, R₅₁, R₅₂, R₅₃, R₅₄, R₅₅, R₅₆, R₅₈, R₅₉ and R₆₀ is independently hydrogen, C₁-C₁₀ alkyl, C₁-C₁₀ heteroalkyl, cycloalkyl, heterocycloalkyl, arylene, or C₁-C₁₀ heteroarylene, each of which except for hydrogen is optionally substituted with one or more R₆₁ and wherein R₅₀ and R₅₁ are optionally joined together to form a heterocycloalkyl or heteroaryl optionally substituted with R₆₁; each of R₆₁ is independently halogen, —OR₆₂, —NR₆₃R₆₄, C₁-C₁₀ alkyl, C₁-C₁₀ heteroalkyl, cycloalkyl, heterocycloalkyl, arylene, or C₁-C₁₀ heteroarylene, wherein the alkyl, heteroalkyl, cycloalkyl, heterocycloalkyl, arylene, or heteroarylene is optionally substituted with one or more R₆₅; each of R₆₂, R₆₃ and R₆₄ is independently hydrogen or C₁-C₁₀ alkyl; and each R₆₅ is independently C₁-C₁₀ alkyl, C₁-C₁₀ heteroalkyl, cycloalkyl, heterocycloalkyl, arylene, or C₁-C₁₀ heteroarylene.

In some embodiments, Y is absent, O, NH, or S. In some embodiments, Y is absent (i.e. Y is a bond). In some embodiments, Y is O. In some embodiments, Y is NH. In some embodiments, Y is S.

The variable x in Formula II is an integer from 0-10. In some embodiments, x is 0. In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10.

The variable y in Formula II is an integer from 0-10. In some embodiments, y is 0. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10.

The variable z in Formula II is an integer from 1-10. In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10.

In some embodiments, x is 0, y is 0, z is 1, and Y is O.

In some embodiments, x is 0, y is 0, z is 1, and Y is S.

In some embodiments, x is 0, y is 0, z is 1, and Y is NH.

In some embodiments, x is 0, y is 0, z is 1, and Y is absent.

In some embodiments, x is 0, y is 0, z is 2, and Y is O.

In some embodiments, x is 0, y is 0, z is 2, and Y is S.

In some embodiments, x is 0, y is 0, z is 2, and Y is NH.

In some embodiments, x is 0, y is 0, z is 2, and Y is absent.

In one aspect the disclosure provides a compound of Formula IIa:

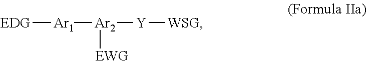

wherein EDG, Ar₁, Ar₂, Y, EWG, and WSG are defined as above for Formula II.

In one aspect the disclosure provides a compound of Formula IIb:

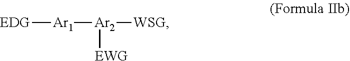

wherein EDG, Ar₁, Ar₂, EWG, and WSG are defined as above for Formula II.

In some embodiments, the compound according to Formula II is selected from a group consisting of:

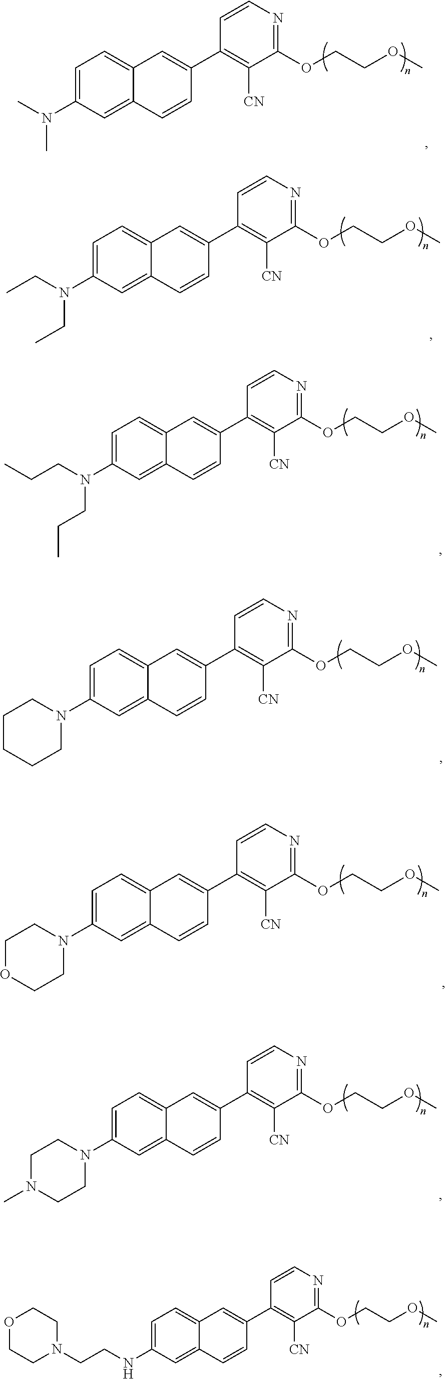

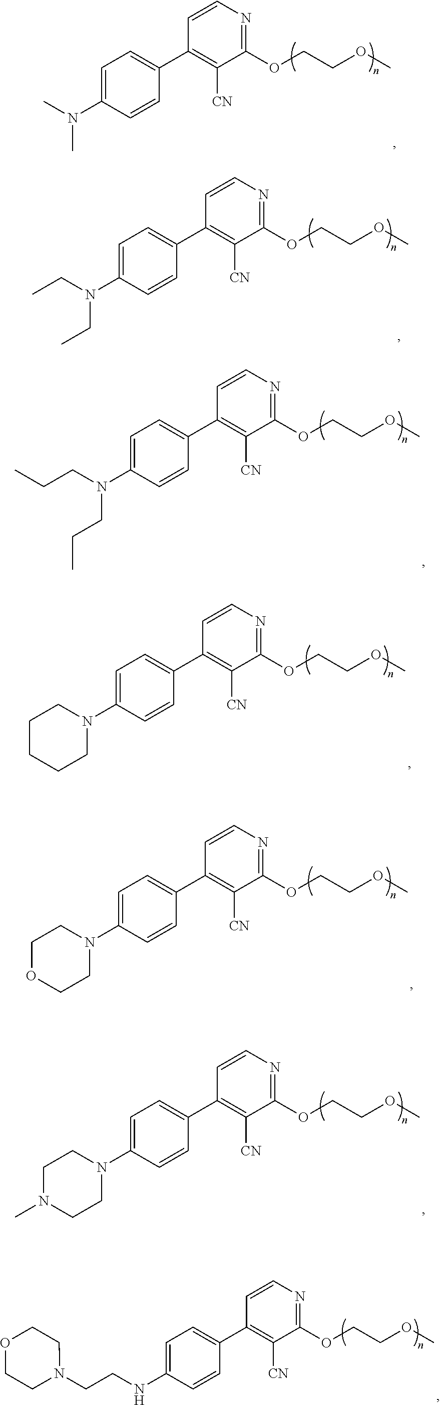

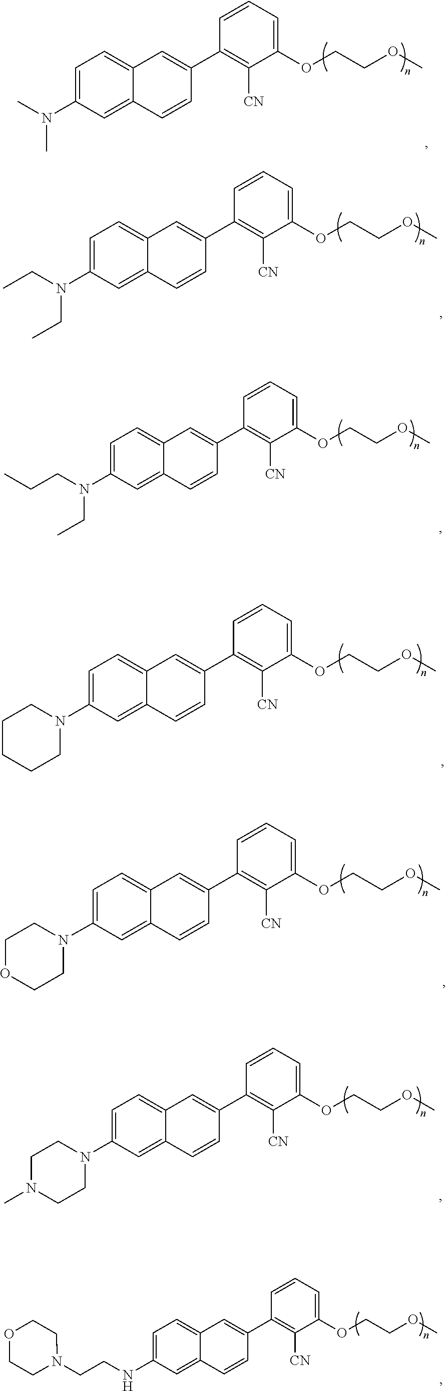

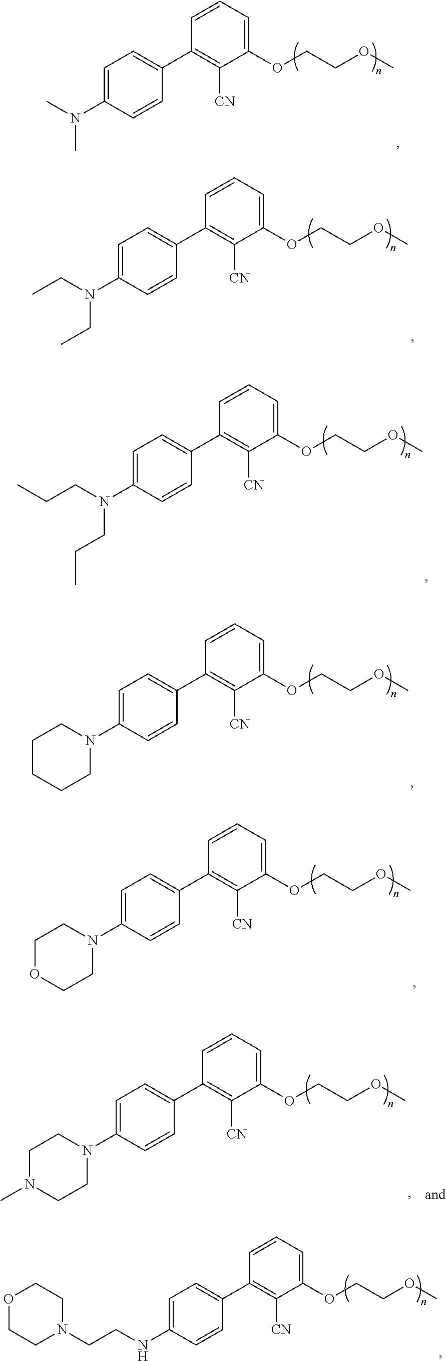

wherein n is an integer with value 0-50. In some embodiments, n is a integer of value 0-10, e.g. n is 0, 1, 2, 3, 4, 5, 6, 7, 8, 9, or 10.

In some embodiments, the compound is selected from a group consisting of:

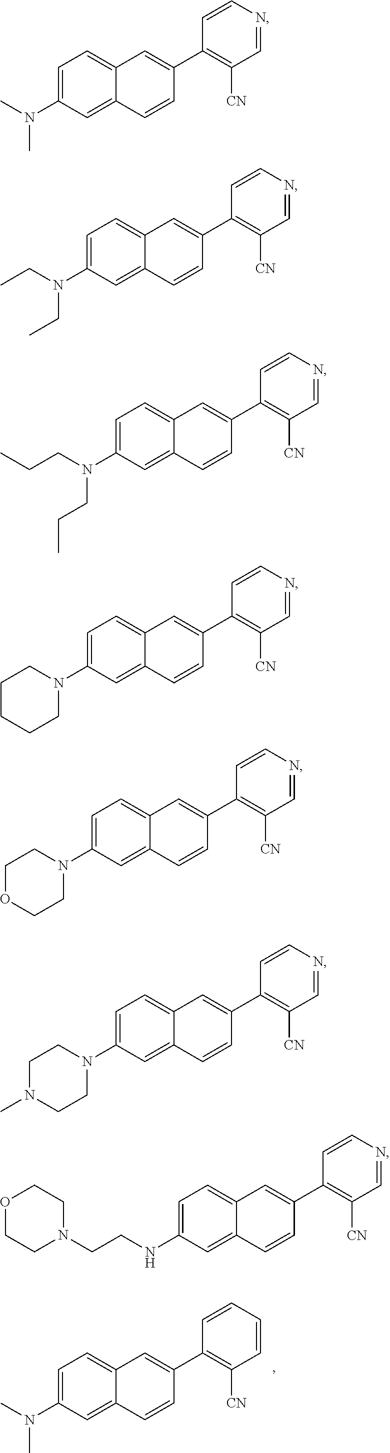

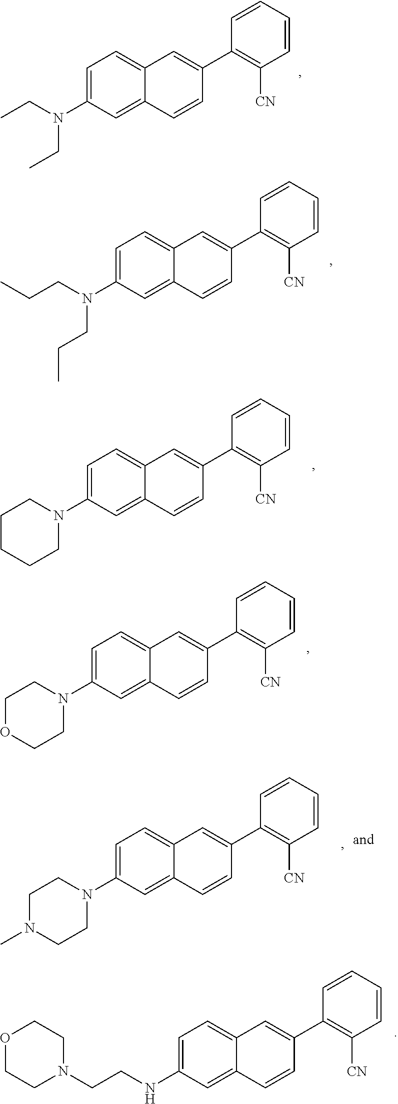 [SMILES: CCN(CC)c1ccc2cc(-c3ccccc3C#N)ccc2c1 ; CCCN(CCC)c1ccc2cc(-c3ccccc3C#N)ccc2c1 ; N#Cc1ccccc1-c1ccc2cc(N3CCCCC3)ccc2c1 ; N#Cc1ccccc1-c1ccc2cc(N3CCOCC3)ccc2c1 ; CN1CCN(c2ccc3cc(-c4ccccc4C#N)ccc3c2)CC1 ; N#Cc1ccccc1-c1ccc2cc(NCCN3CCOCC3)ccc2c1]

In some embodiments, the compound is selected from a group consisting of

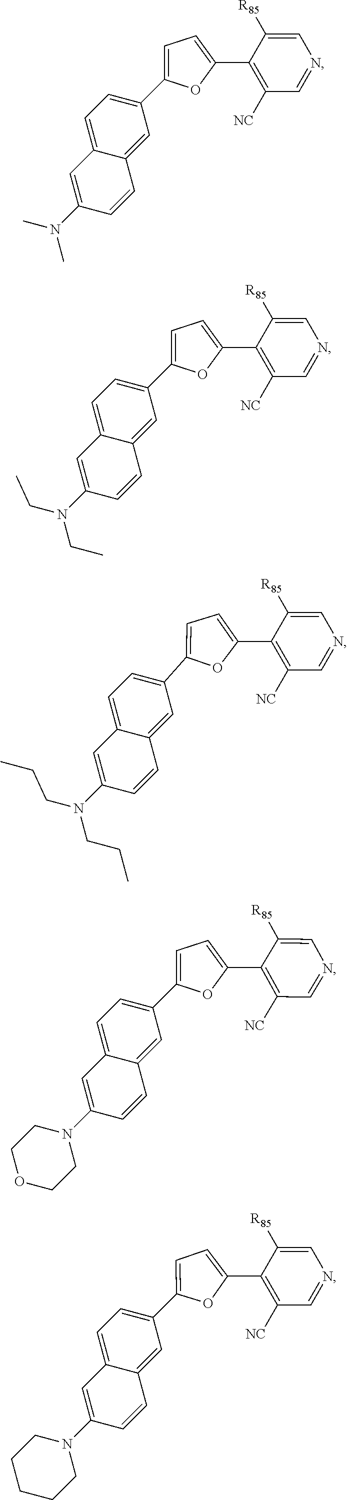

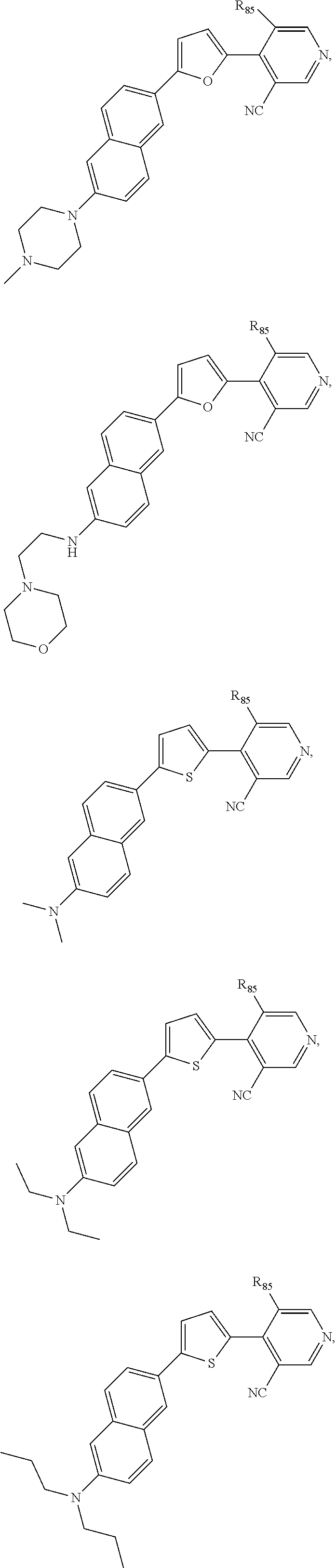

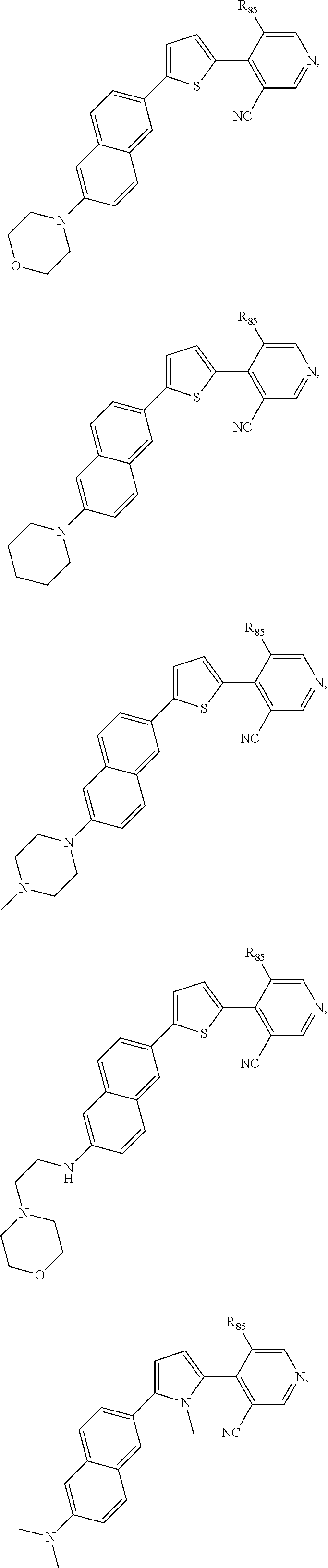

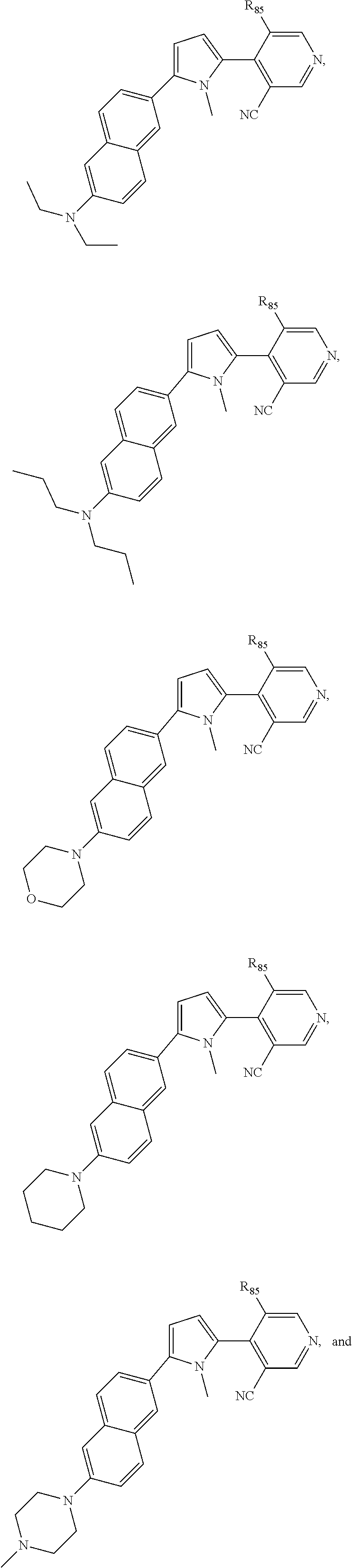

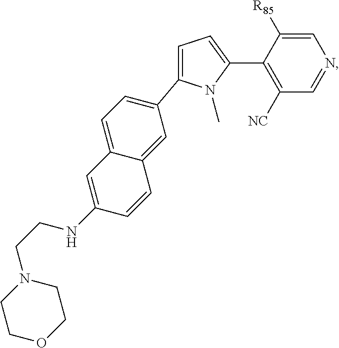

wherein R₈₅ is H or CN.

In some embodiments, the compound is selected from a group consisting of

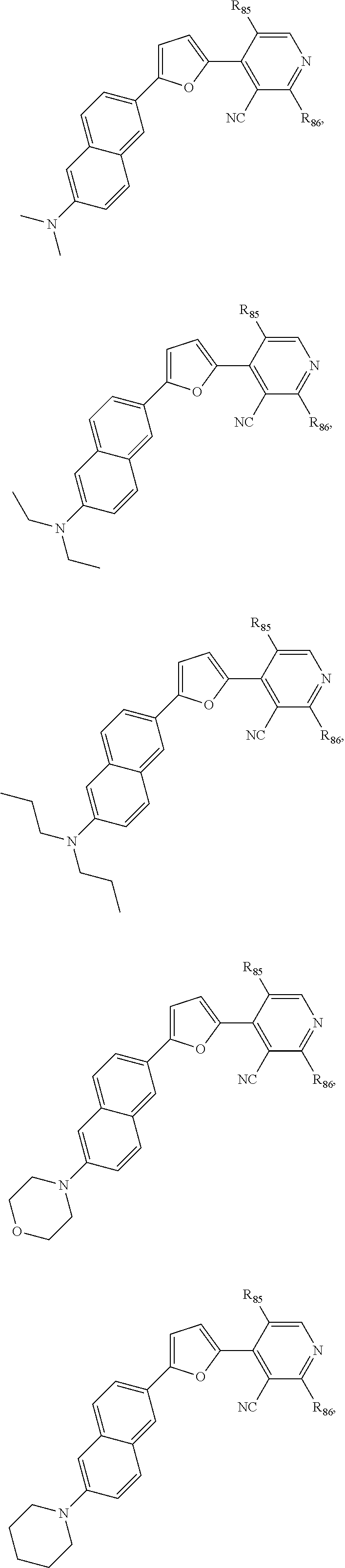

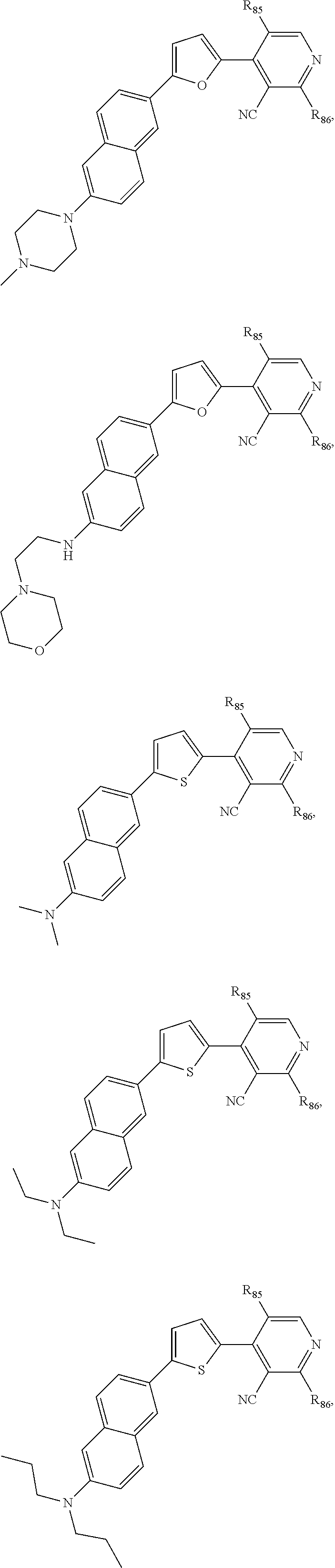

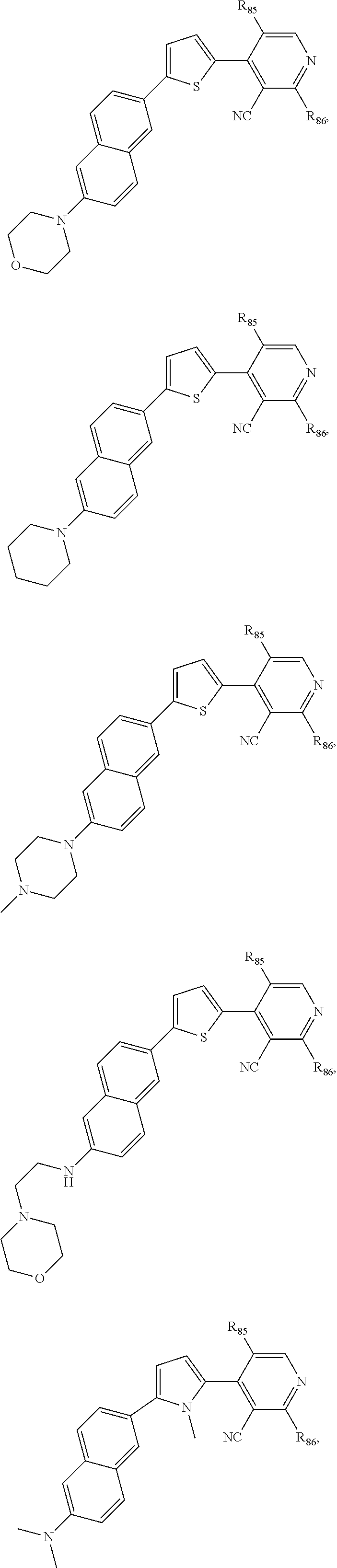

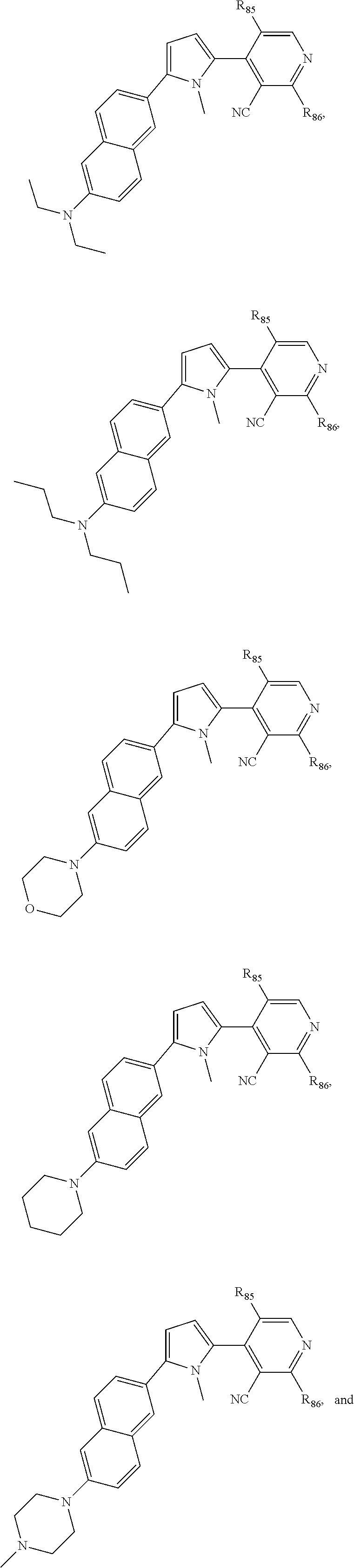

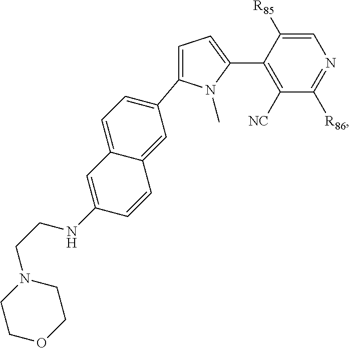

wherein R₈₅ is H or CN and R₈₆ is

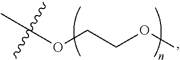

wherein n is an integer with value 0-50. In some embodiments, n is a integer of value 0-10, e.g. n is 0, 1, 2, 3, 4, 5, 6, 7, 8, 9, or 10.

In some embodiments, the compound is selected from a group consisting of

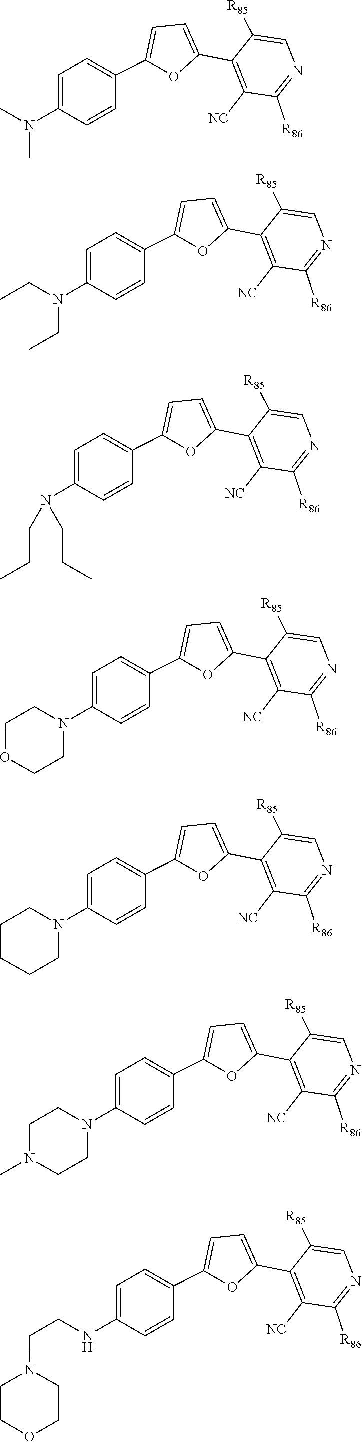

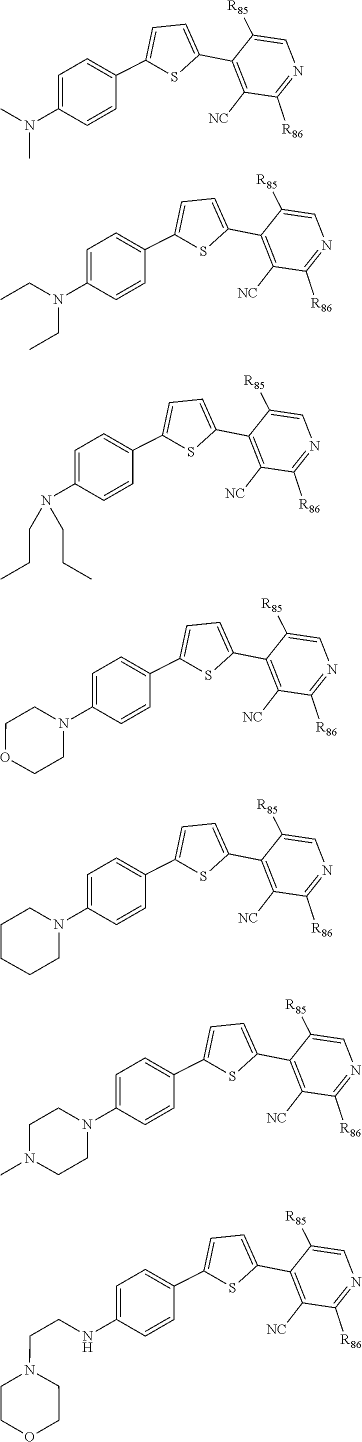

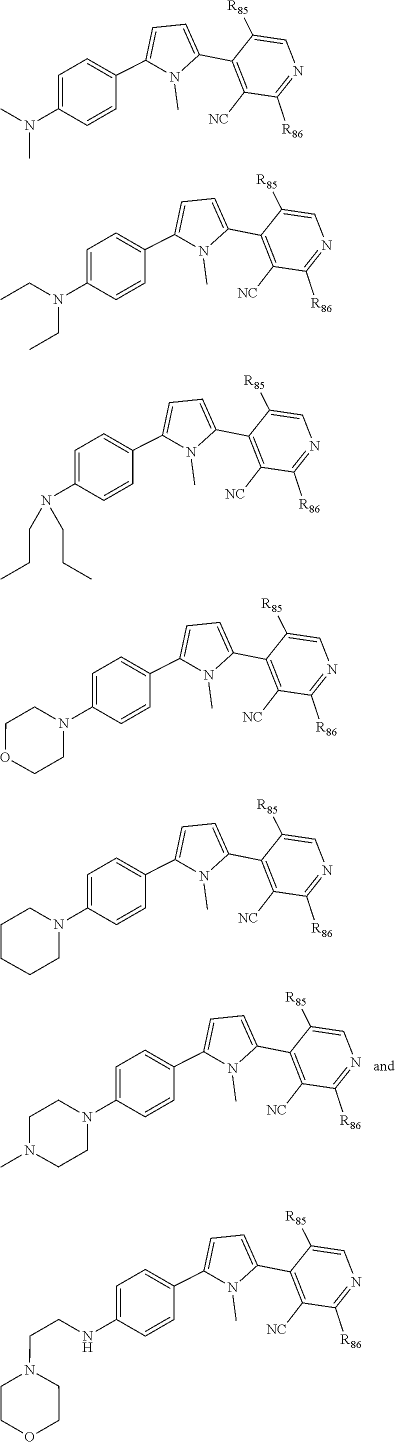

wherein R₈₅ is H or CN and R₈₆ is H.

In some embodiments, the compound is selected from a group consisting of

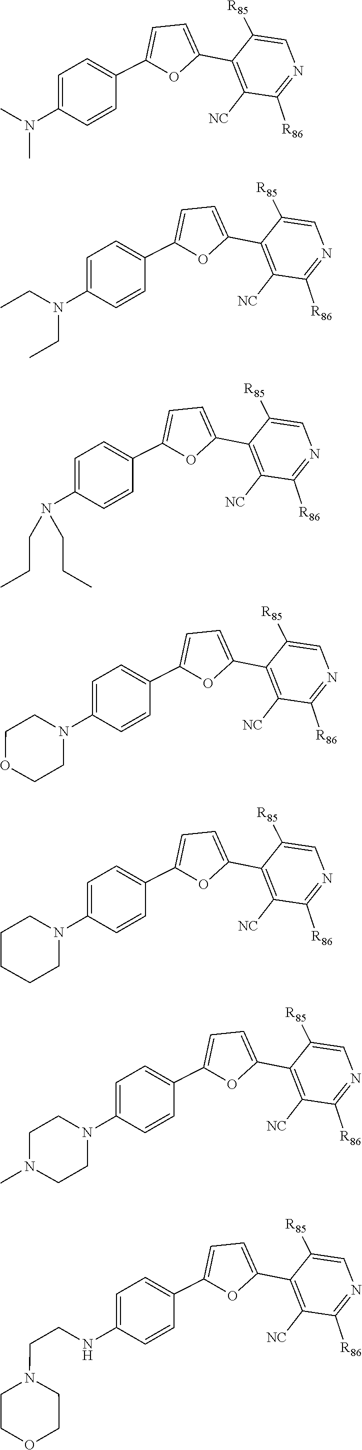

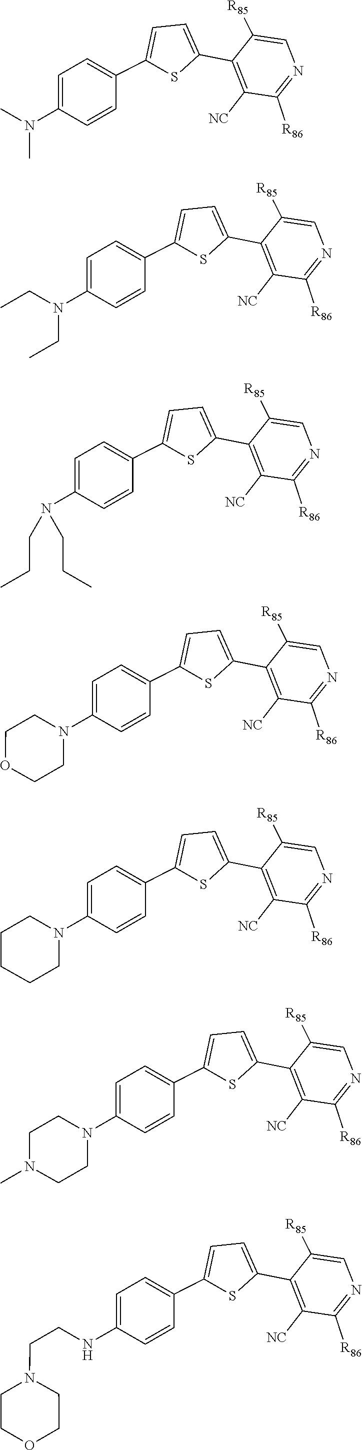

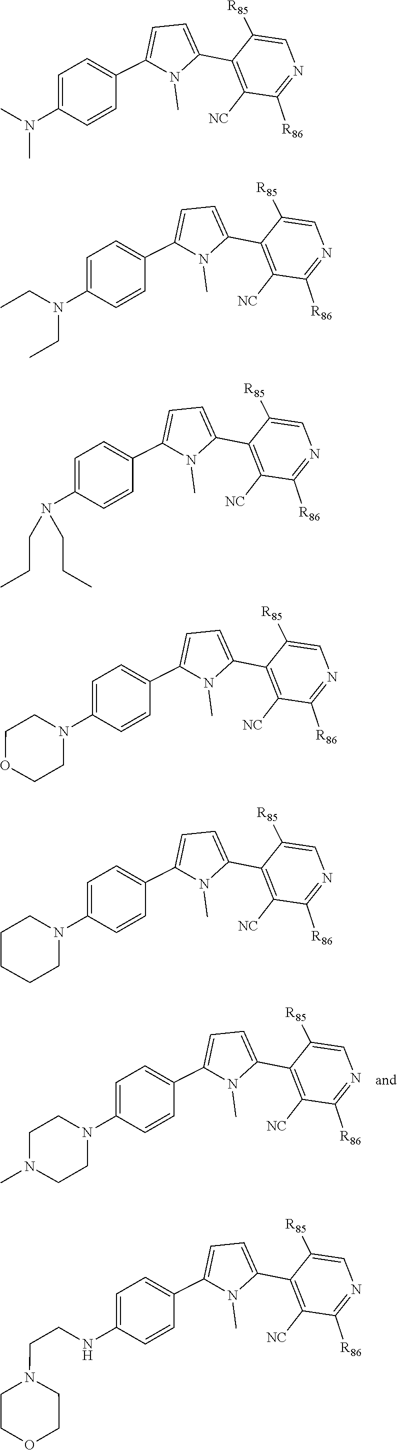

wherein R₈₅ is H or CN and R₈₆ is

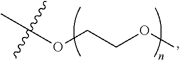

wherein n is an integer with value 0-50. In some embodiments, n is a integer of value 0-10, e.g. n is 0, 1, 2, 3, 4, 5, 6, 7, 8, 9, or 10.

In some embodiments, the compound is:

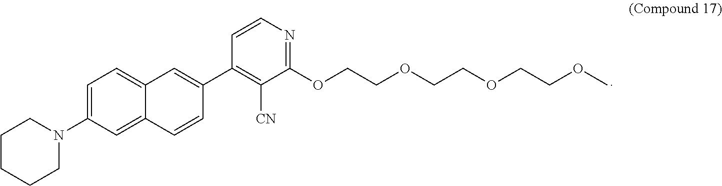 [SMILES: COCCOCCOCCOc1nccc(-c2ccc3cc(N4CCCCC4)ccc3c2)c1C#N]

In some embodiments, the compound is 2-(2-(2-(2-methoxyethoxy)ethoxy)ethoxy)-4-(6-(piperidin-1-yl)naphthalen-2-yl)nicotinonitrile.

In some embodiments, the compound is:

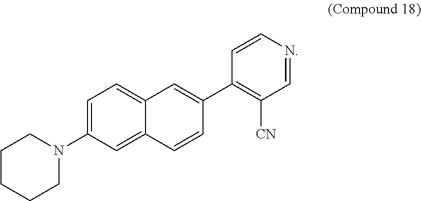 [SMILES: N#Cc1cnccc1-c1ccc2cc(N3CCCCC3)ccc2c1]

In some embodiments, the compound is 4-(6-(piperidin-1-yl)naphthalen-2-yl)nicotinonitrile.

In some embodiments, the compound is selected from the following:

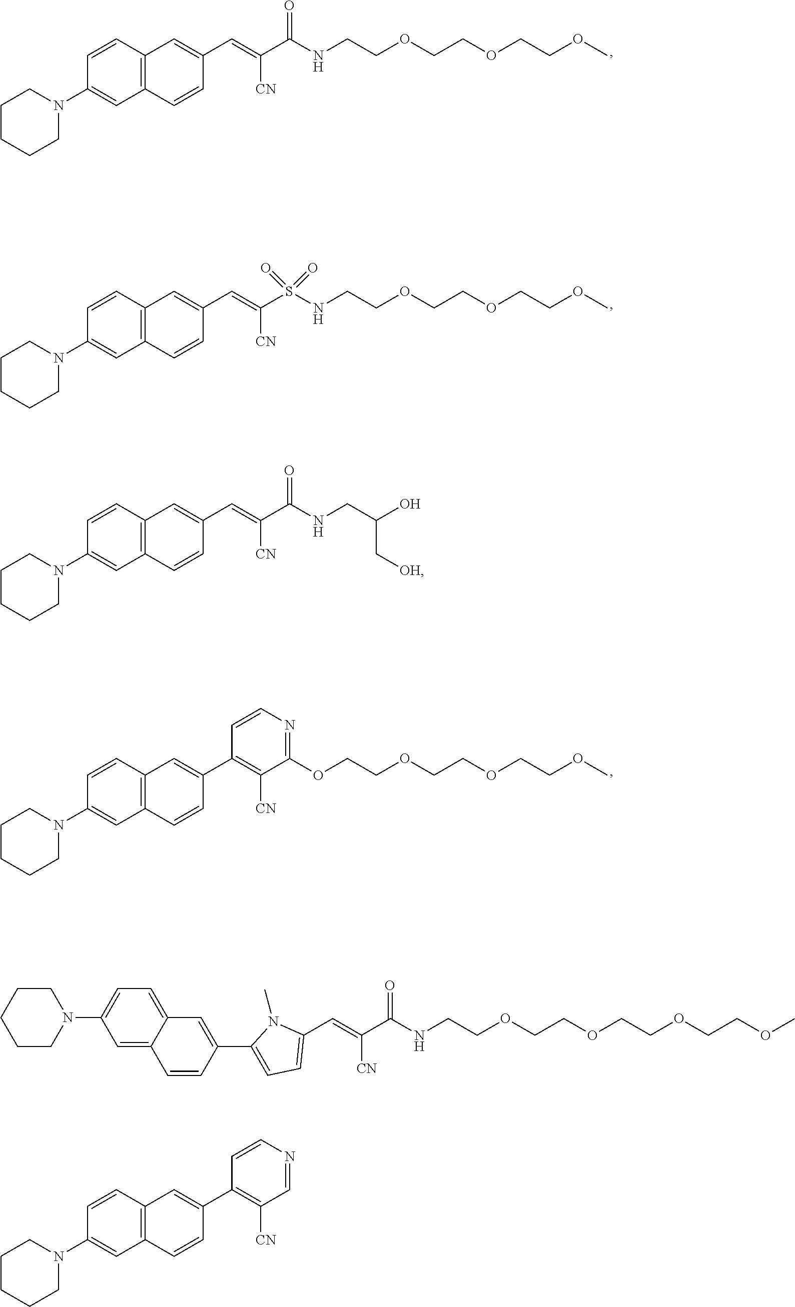 [SMILES: COCCOCCOCCNC(=O)/C(C#N)=C/c1ccc2cc(N3CCCCC3)ccc2c1 ; COCCOCCOCCNS(=O)(=O)/C(C#N)=C/c1ccc2cc(N3CCCCC3)ccc2c1 ; N#C/C(=C\c1ccc2cc(N3CCCCC3)ccc2c1)C(=O)NCC(O)CO ; COCCOCCOCCOc1nccc(-c2ccc3cc(N4CCCCC4)ccc3c2)c1C#N ; COCCOCCOCCOCCNC(=O)/C(C#N)=C/c1ccc(-c2ccc3cc(N4CCCCC4)ccc3c2)n1C ; N#Cc1cnccc1-c1ccc2cc(N3CCCCC3)ccc2c1]

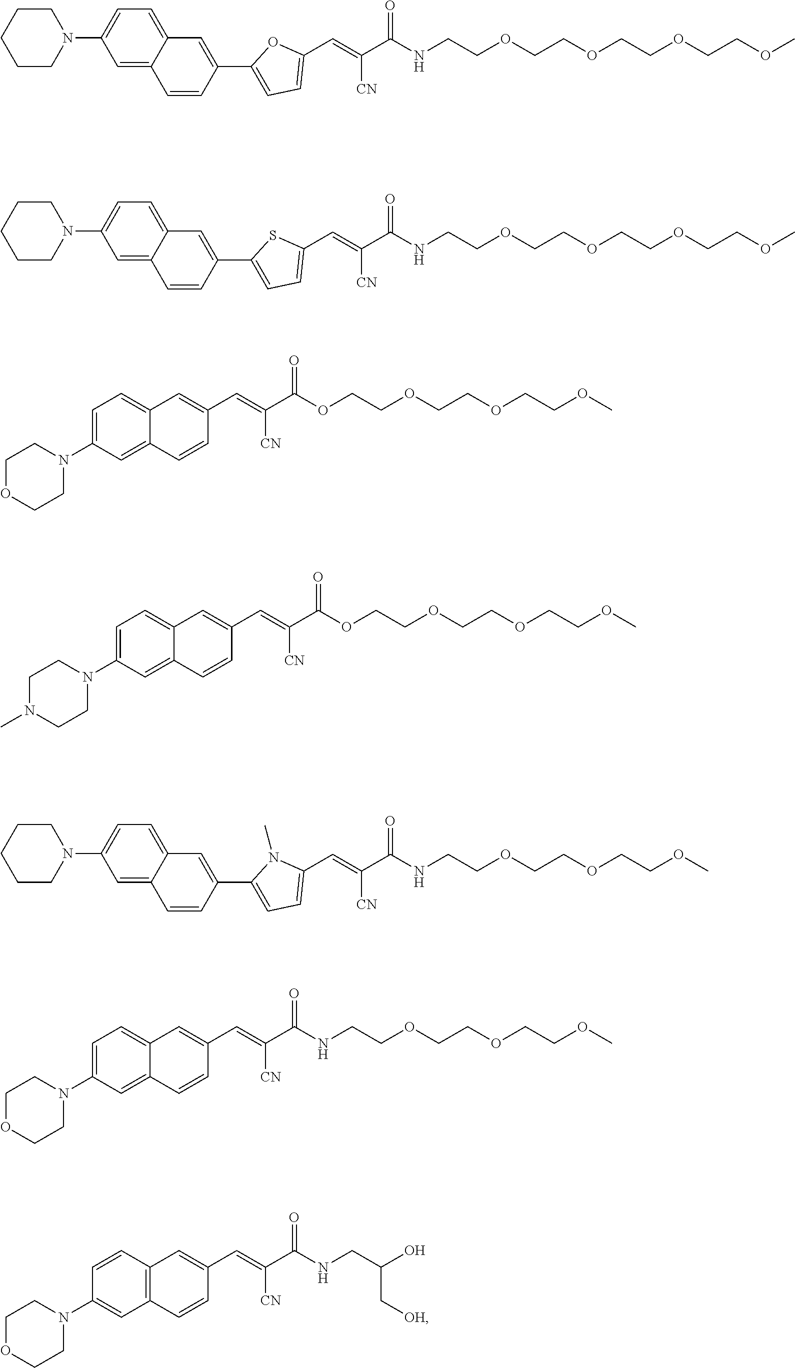

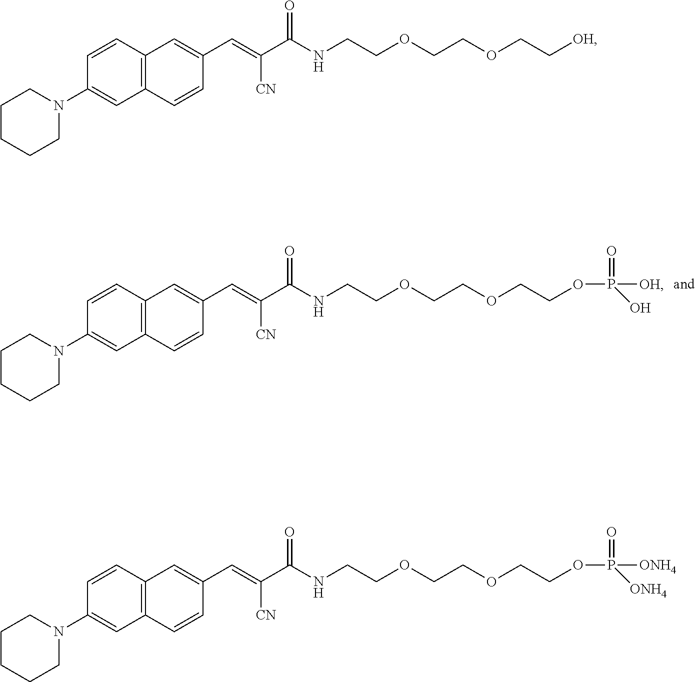

or a salt or solvate thereof.

### Neurological Diseases and Disorders and Treatment Thereof

Provided herein are methods for determining whether a patient suffers from a neurological disease or disorder, comprising detecting the presence of a phosphorylated tau protein in a tissue of the patient, wherein the detecting comprises contacting the phosphorylated tau protein with a compound described herein. The compound may be a compound of Formula I, II, Ia, Ib, Ic, Id, or Ie. In some embodiments, the compound is a compound of Formula Ic. In some embodiments, the compound is Compound 1, 2, 3, 4, or 5. The contacting may be in vivo. The tissue may be an eye tissue. In some embodiments, the neurological disease or disorder is selected from an age-related disease or disorder, a genetic disease or disorder, an injury-related disease or disorder, and a psychiatric disease or disorder. In some embodiments, the age-related disease or disorder is selected from Parkinson's dementia, vascular dementia, and Amyotrophic lateral sclerosis, wherein the genetic disease or disorder is Down syndrome, wherein the injury-related disease or disorder is selected from Traumatic Brain Injury and Chronic Traumatic Encephalopathy, and wherein the psychiatric disease or disorder is selected from schizophrenia and depression. The neurological disease or disorder may be a tauopathy. In some embodiments, the neurological disease or disorder is Alzheimer's disease or Chronic Traumatic Encephalopathy (CTE).

The neurological disease or disorder may be a tauopathy. Tauopathies are a class of neurological diseases associated with the pathological aggregation of tau protein in neurofibrillary or gliofibrillary tangles in the human brain. Tangles may be formed by hyperphosphorylation of tau, causing the tau protein to dissociate from microtubules and form aggregates in an insoluble form. The aggregations of hyperphosphorylated tau protein may also be referred to as paired helical filaments. The precise mechanism of tangle formation is not completely understood, and it is still controversial as to whether tangles are a primary causative factor in the disease or play a more peripheral role. Tauopathy has been found in many neurological disorders, such as posttraumatic degeneration, infections, metabolic diseases, and motor neuron degeneration. The spatial distribution, temporal appearance, and structural changes of tau proteins manifest differently among various neurological diseases. AD patients have twisted, hyperphosphorylated, and single nonperiodical tau filaments, whereas patients having progressive supernuclear palsy and frontotemporal dementia (FTD) tend to have only straight tau filaments. Tauopathies are often overlapped with synucleinopathies, possibly due to interaction between the synuclein and tau proteins. Non-Alzheimer's tauopathies are sometimes grouped together as “Pick's complex” due to their association with frontotemporal dementia, or frontotemporal lobar degeneration. A marker of tau hyperphosphorylation is tau pS422. Chronic traumatic encephalopathy (CTE) is associated with repetitive mild traumatic brain injury (mTBI), and bears many similarities with tauopathies, including hyperphosphorylation and aggregation of tau, for example, as neurofibrillary tangles (NFTs).

The neurological disease or disorder may be a neurodegenerative disease or disorder. In some embodiments, the neurological disease or disorder is Alzheimer's disease (AD). AD is considered a secondary tauopathy. Alzheimer's disease is characterized by symptoms of memory loss in the early stages of the disease. Neurofibrillary tangles were an early descriptor of AD. When tau becomes hyperphosphorylated, the protein dissociates from the microtubules in axons. Then, tau may become misfolded and begin to aggregate, which may form neurofibrillary tangles (NFT). Microtubules also destabilize when tau is dissociated, and the combination of the neurofibrillary tangles and destabilized microtubules result in disruption of processes such as axonal transport and neural communication. The degree of NFT involvement in AD is defined by Braak stages. Braak stages I and II are used when NFT involvement is confined mainly to the transentorhinal region of the brain; stages III and IV when limbic regions such as the hippocampus become involved; stages V and VI when extensive neocortical involvement is indicated. AD is also classified as an amyloidosis because of the presence of senile plaques. Additionally, certain Apoε4 carriers are at greater risk of developing AD. APOε4 is believed to be less efficient than other isoforms at clearing Ac, and thus may be correlated with greater amyloid burden, tau phosphorylation, synaptotoxicity, and reduced synaptic density. Having experienced a traumatic brain injury (TBI) is another risk factor for developing AD, and studies indicate that those who experience a TBI have a significantly increased risk of AD.

As AD advances, symptoms include confusion, long-term memory loss, paraphasia, loss of vocabulary, aggression, irritability and/or mood swings. In more advanced stages of the disease, there is loss of bodily functions. Patients with Alzheimer's Disease (AD) demonstrate many characteristic neuropathies such as increased oxidative stress, mitochondrial dysfunction, synaptic dysfunction, disruption of calcium homeostasis, deposition of senile plaques and neurofibrillary tangles, and atrophy of the brain. AD related disorders include senile dementia of AD type (SDAT), frontotemporal dementia (FTD), vascular dementia, mild cognitive impairment (MCI) and age-associated memory impairment (AAMI). In some embodiments, determining whether a patient suffers from Alzheimer's disease, comprising detecting the presence of a phosphorylated tau protein in a tissue of the patient, wherein the detecting comprises contacting the phosphorylated tau protein with a compound described herein.

In some embodiments, the neurological disease or disorder disease is frontotemporal lobar degeneration (FTLD) (e.g., FTLD-tau, FTLD-TDP, or FTLD-FUS). In some embodiments, the neurological disease or disorder is frontotemporal lobe dementia. In some embodiments, the neurological disease or disorder includes memory loss. In some embodiments, the neurological disease or disorder is age-related memory loss. In some embodiments, the neurological disease or disorder is FTLD-TDP Type A. In some embodiments, the neurological disease or disorder is FTLD-TDP Type B. In some embodiments, the neurological disease or disorder is FTLD-TDP Type C. In some embodiments, the neurological disease or disorder is FTLD-TDP Type D.

In some embodiments, the neurological disease or disorder is Parkinson's disease. In some embodiments, the neurological disease or disorder is Parkinson's dementia. In some embodiments, the neurological disease or disorder is related to (e.g. characterized by) an accumulation of amyloid plaques. In some embodiments, a patient having a neurological disease or disorder has suffered a traumatic brain injury before, during, or after the onset of the neurological disease or disorder. In some embodiments, the neurological disease or disorder includes a neuronal impairment. A neuronal impairment may include atrophy or other decrease in the effective functioning of the neuron. For example, it is known that Alzheimer's disease presents with neuronal impairment, especially in cortical neurons, e.g., hippocampal neurons and neurons in proximity to the hippocampus.

In some embodiments, the neurological disease or disorder is traumatic axonal injury (TAI), traumatic brain disorder (TBD), dementia (e.g., general dementia), frontotemporal dementia with parkinsonism linked to chromosome 17 (FTDP-17), primary age-related tauopathy (PART), neurofibrillary tangle-predominant senile dementia, progressive supranuclear palsy (PSP), corticobasal degeneration, Lytico-Bodig disease (Parkinson-dementia complex of Guam), ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, Pick's disease, corticobasal degeneration, argyrophilic grain disease (AGD), or corticobasal degeneration.

The neurological disease or disorder may be an injury-related condition such as Traumatic Brain Injury (TBI) or Chronic Traumatic Encephalopathy (CTE). TBI is a chronic disease from damage to the brain caused by an external force, such as a bump, blow, jolt, rapid acceleration or deceleration, or penetration by a projectile. Injury leading to TBI may produce diminished or altered states of consciousness, resulting in temporary or permanent impairment in cognition, sensorimotor, and psychosocial function. CTE is a progressive degenerative disease found in people who have suffered repetitive brain trauma, including hits to the head that did not result in TBI symptoms. Physical aspects of CTE include shrinking of the brain, atrophy of the frontal and temporal lobes, enlargement of the ventricles, atrophy of the hippocampus, thalamus, brainstem and cerebellum. Individuals with CTE may have symptoms of dementia, memory loss, aggression, confusion, depression and suicidal ideations that may occur many years after the injuries.

The neurological disease or disorder may be of the eye, for example, glaucoma, ocular hypertension, macular degeneration, diabetic retinopathy, age-related macular degeneration (AMD) or retinitis pigmentosa.

Additional examples of a neurological disease or disorder include Alexander's disease, Alper's disease, depression, perinatal asphyxia, Parkinson's disease dementia (“PD dementia”), amyotrophic lateral sclerosis, ataxia telangiectasia, Batten disease (also known as Spielmeyer-Vogt-Sjogren-Batten disease), spongiform encephalopathy (e.g., bovine spongiform encephalopathy (mad cow disease), Kuru, Creutzfeldt-Jakob disease, fatal familial insomnia, Canavan disease, Cockayne syndrome, corticobasal degeneration, fragile X syndrome, frontotemporal dementia, Gerstmann-Straussler-Scheinker syndrome, Huntington's disease, HIV-associated dementia, Kennedy's disease, Krabbe's disease, Lewy body dementia, Machado-Joseph disease (Spinocerebellar ataxia type 3), multiple sclerosis, multiple system atrophy, narcolepsy, neuroborreliosis, Pelizaeus-Merzbacher Disease, primary lateral sclerosis, prion diseases, Refsum's disease, Sandhoff s disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, Tabes dorsalis, drug-induced Parkinsonism, progressive supranuclear palsy, corticobasal degeneration, multiple system atrophy, idiopathic Parkinson's disease, autosomal dominant Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), and mitochondrial Parkinson's disease.

Upon determination of a neurological disease or disorder in a patient, certain procedures can be provided to treat or ameliorate the symptoms of the neurological disease or disorder, or to slow or halt the progression thereof. Once a neurological disease or disorder is diagnosed, the progression of the disease or disorder may also be monitored by the methods described herein. Once diagnosed, the treating physician may also suggest additional treatments as known to practitioners, including those described herein.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) ameliorating, slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a patient (including a human) who is at risk or has a family history of the disease or condition.

“Patient” refers to an animal, such as a mammal (including a human), that has been or will be the object of treatment, observation or experiment. The methods described herein may be useful in human therapy and/or veterinary applications. In some embodiments, the patient is a mammal. In one embodiment, the patient is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a patient, to provide a therapeutic benefit such as amelioration of symptoms or slowing of disease progression. For example, a therapeutically effective amount may be an amount sufficient to decrease a symptom of a disease or condition of a neurological disease or disorder. The therapeutically effective amount may vary depending on the patient, and disease or condition being treated, the weight and age of the patient, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human patients. Such properties may be examined using commonly known methods to those skilled in the art.

### Detection of Phosphorylated Tau

Provided herein are methods for diagnosis of a neurological disease or disorder, comprising administering to a tissue of the patient a compound described herein. The compound may be a compound of Formula I, II, Ia, Ib, Ic, Id, or Ie. In some embodiments, the compound is a compound of Formula Ic. In some embodiments, the compound is Compound 1, 2, 3, 4, or 5. The method may comprise detecting a binding of the compound to a phosphorylated tau protein. The binding, upon activation by a light, may cause emission of a detectable signal. The signal may be a fluorescent or infrared signal. The administration may be intravenous administration or be localized at the retina of the eye.

Detection of a phosphorylated tau protein can be made with probes that can selectively bind to the phosphorylated tau, which is herein referred to as a phosphorylated tau probe. The probe comprises a compound described herein.

In one embodiment, a phosphorylated tau probe, for example, a compound described herein, when bound to a phosphorylated tau, can be detected through its emitted fluorescent signal, upon activation by a laser light. Examples of phosphorylated tau-binding compounds are described herein.

In situ detection of binding of a phosphorylated tau-binding probe, for example, a compound described herein, to a phosphorylated tau protein in the retina of the patient can be facilitated with a retinal imaging device, which is preferably handheld or portable. The retinal imaging device can include a lens and an image sensor, and optionally a laser light source. When the light source emits laser light to the tissue, for example, the retina, if phosphorylated tau is accumulated there and has bound to a phosphorylated tau-binding probe, for example, a compound described herein, the accumulation can be readily detected and quantitated by the lens and image sensor that collects and senses a fluorescent signal.

The methods for determining whether a patient suffers from a neurological disease or disorder, may be conducted in any manner described herein. In some embodiments, the contacting, upon activation by a light, causes emission of detectable signal. In some embodiments, the detectable signal is a fluorescent signal. In some embodiments, the phosphorylated tau protein includes at least three moles of phosphate per mole of protein. In some embodiments, the phosphorylated tau protein is a three-repeat tau, a four-repeat tau, or a combination thereof.

### Administration and Pharmaceutical Compositions

In some embodiments, a compound described herein is administered to the eye. In some embodiments, a pharmaceutical composition of the disclosure administered to eye is delivered to the retina, intraocular space, ocular surface, interconnecting innervation, conjunctiva, lacrimal glands, or meibomian glands. In some embodiments, the compounds are administered topically to the eye. In some embodiments, the compound is administered as an eye drop. The administration may be topical administration.

The compound can also be formulated for intravenous and subcutaneous use, without limitation. The intravenous administration can be bolus administration or continuous injection.

The compound may be effective over a wide dosage range. In some embodiments, in the application to adult humans, dosages from 0.01 to 1000 mg, from 0.5 to 100 mg, from 1 to 50 mg per day, and from 5 to 40 mg per day are examples of dosages that are used. An exemplary dosage is 10 to 30 mg per day. In the applications to juveniles, the dosage may be the same or less than the adult dose. In some embodiments the effective amount of the compound corresponds to about 50-500 mg of compound per adult patient. The exact dosage will depend upon the route of administration, the form in which the compound is administered, the patient to be treated, the body weight of the patient to be treated, and the preference and experience of the attending physician.

In some embodiments the effective amount of the compound corresponds to about 0.01-1000 mg of compound per human patient per dosage. In some embodiments, the effective dose of compound is be 50-500 mg per human per dosage. In some embodiments the effective amount corresponds to about 0.01-100 mg, 0.01-200 mg, 0.01-300 mg, 0.01-400 mg, 0.01-500 mg, 0.01-600 mg, 0.01-700 mg, 0.01-800 mg, 0.01-900 mg, 0.01-1000 mg, 0.1-100 mg, 0.1-200 mg, 0.1-300 mg, 0.1-400, 0.1-500 mg, 0.1-600 mg, 0.1-700 mg, 0.1-800 mg, 0.1-900 mg, 0.1-1000 mg, 1-100 mg, 1-200 mg, 1-300 mg, 1-400 mg, 1-500 mg, 1-600 mg, 1-700 mg, 1-800 mg, 1-900 mg, 100-200 mg, 100-300 mg, 100-400 mg, 100-500 mg, 100-600 mg, 100-700 mg, 100-800 mg, 100-900 mg, 100-1000 mg, 200-300 mg, 200-400 mg, 200-500 mg, 200-600 mg, 200-700 mg, 200-800 mg, 200-900 mg, 200-1000 mg, 300-400 mg, 300-500 mg, 300-600 mg, 300-700 mg, 300-800 mg, 300-900 mg, 300-1000 mg, 400-500 mg, 400-600 mg, 400-700 mg, 400-800 mg, 400-900 mg, 400-1000 mg, 500-600 mg, 500-700 mg, 500-800 mg, 500-900 mg, 500-1000 mg, 600-700 mg, 600-800 mg, 600-900 mg, 600-1000 mg, 700-800 mg, 700-900 mg, 700-1000 mg, 800-900 mg, 800-1000 mg or about 900-1000 mg per human per dosage. In some embodiments, the effective amount corresponds to about 50-100 mg, 50-400 mg, 50-500 mg, 100-200 mg, 100-300 mg, 100-400 mg, 100-500 mg, 200-300 mg, 200-400 mg, 200-500, 300-400 mg, 300-500 mg, or 400-500 mg per adult human per dosage.

In some embodiments, the compound is administered in a single dose. In some embodiments, the compound is administered in multiple doses. In some embodiments, dosing is about once, twice, three times, four times, five times, six times, or more than six times per day. In some embodiments, dosing is about once a month, once every two weeks, once a week, or once every other day. In another case the compound and another agent are administered together about once per day to about 6 times per day. In some embodiments the administration of the compound and an agent continues for less than about 7 days. In yet another case the administration continues for more than about 6, 10, 14, 28 days, two months, six months, or one year. In some embodiments, continuous dosing is achieved and maintained as long as necessary.

In some embodiments, the compound is administered one to ten times, one to four times, or once a day. In some embodiments, the compound is administered 1, 2, 3, 4, 5, 6, 7, 8, 9, or 10 times a day. In some embodiments, the compound is administered as drops. In some embodiments, the size of the drop administered is in the range of about 10-100 μL, about 10-90 μL, about 10-80 μL, about 10-70 μL, about 10-60 μL, about 10-50 μL, about 10-40 μL, about 10-30 μL, about 20-100 μL, about 20-90 μL, about 20-80 μL, about 20-70 μL, about 20-60 μL, about 20-50 μL, about 20-40 μL, or about 20-30 μL. One example of the disclosure administers a drop in the range of about 10 to about 30 μL. One example of the disclosure administers a drop in the range of about 10 to about 100 μL. One example of the disclosure administers a drop in the range of about 20 to about 50 μL. One example of the disclosure administers a drop in the range of about 20 to about 40 μL. One example of the disclosure administers a drop in the range of about 10 to about 60 μL. In some embodiments, the eye formulations of the disclosure is administered several drops per time, for example 1-3 drops per time, 1-3 drops per time, 1-4 drops per time, 1-5 drops per time, 1-6 drops per time, 1-7 drops per time, 1-8 drops per time, 1-9 drops per time, 1-10 drops per time, 3-4 drops per time, 3-5 drops per time, 3-6 drops per time, 3-7 drops per time, 3-8 drops per time, 3-9 drops per time, 3-10 drops per time, 5-6 drops per time, 5-7 drops per time, 5-8 drops per time, 5-9 drops per time, 5-10 drops per time, 7-8 drops per time, 7-9 drops per time or 9-10 drops per time. In one example, the formulations of the disclosure are administered about one drop per time and 1-6 times per day.

### Pharmaceutical Compositions/Formulations

In some embodiments, the compound described herein is formulated into a pharmaceutical composition. In some embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound as described herein and a pharmaceutically acceptable diluent(s), excipient(s), or carrier(s). In certain cases, the compound described herein is administered as pharmaceutical compositions in which one or more compounds, are mixed with other active ingredients, as in combination therapy. In specific cases, the pharmaceutical compositions include one or more compounds as described herein.

A pharmaceutical composition, as used herein, refers to a mixture of a compound described herein, with other chemical components, such as carriers, stabilizers, diluents, dispersing agents, suspending agents, thickening agents, and/or excipients. In certain cases, the pharmaceutical composition facilitates administration of the compound to an organism. In some embodiments for practicing the methods of treatment or use provided herein, therapeutically effective amounts of one or more compounds described herein are administered in a pharmaceutical composition to a mammal having a disease or condition to be detected, diagnosed or treated. In specific cases, the mammal is a human. In certain cases, therapeutically effective amounts vary depending on the severity of the disease, the age and relative health of the patient, the potency of the compound used and other factors. The compounds described herein are used singly or in combination with one or more therapeutic agents as components of mixtures.

In some embodiments, the one or more compounds is formulated in an aqueous solution. In specific cases, the aqueous solution is selected from, by way of example only, a physiologically compatible buffer, such as Hank's solution, Ringer's solution, aqueous acetate buffer, aqueous citrate buffer, aqueous carbonate buffer, aqueous phosphate buffer or physiological saline buffer.

In some embodiments, the compounds described herein are formulated for ocular administration. In some embodiments, the ocular formulations is liquid (in form of solutions, suspensions, powder for reconstitution, sol to gel systems), semi solids (ointments and gels), solids (ocular inserts), and intraocular dosage forms (injections, irrigating solutions and implants).

Provided herein are ophthalmic formulations comprising the compounds described herein and an ophthalmologically acceptable component. The ophthalmic formulation may be administered in any form suitable for ocular drug administration, e.g., as a solution, suspension, ointment, gel, liposomal dispersion, colloidal microparticle suspension, or the like, or in an ocular insert, e.g., in an optionally biodegradable controlled release polymeric matrix.

By a “pharmaceutically acceptable” or “ophthalmologically acceptable” component is meant a component that is not biologically or otherwise undesirable, i.e., the component may be incorporated into an ophthalmic formulation of the disclosure and administered topically to a patient's eye without causing any undesirable biological effects or interacting in a deleterious manner with any of the other components of the formulation composition in which it is contained. When the term “pharmaceutically acceptable” is used to refer to a component other than a pharmacologically active agent, it is implied that the component has met the required standards of toxicological and manufacturing testing or that it is included on the Inactive Ingredient Guide prepared by the U.S. Food and Drug Administration.

Ophthalmic formulations may be adapted for topical administration to the eye in the form of a suspension or emulsion. The ophthalmic formulation may include an ophthalmologically acceptable carrier. Such carriers include, for example, water, mixtures of water, for example, phosphate buffer, boric acid, sodium chloride, and sodium borate, and water-miscible solvents such as lower alcohols, aryl alcohols, polyalkylene glycols, carboxymethylcellulose, polyvinylpyrrolidone, and isopropyl myristate. The ophthalmic formulation may also include one or more excipients such as emulsifying agents, preserving agents, wetting agents, bodying agents. For example, the ophthalmic formulation may include polyethylene glycols 200, 300, 400 and 600, carbowaxes 1,000, 1,500, 4,000, 6,000 and 10,000, antibacterial components such as quaternary ammonium compounds, phenylmercuric salts, thimerosal, methyl and propyl paraben, benzyl alcohol, phenyl ethanol, buffering agents such as sodium borate, sodium acetates, gluconate buffers, and other agents such as sorbitan monolaurate, triethanolamine, oleate, polyoxyethylene sorbitan monopalmitylate, dioctyl sodium sulfosuccinate, monothioglycerol, thiosorbitol, and ethylenediamine tetracetic acid. The ophthalmic formulation may be isotonic. The ophthalmic formulation may also include a surfactant or a stabilizer. Surfactants include Carbopol®. Stabilizers include sodium bisulfate, sodium metabisulfate and sodium thiosulfate.

The formulation may include an effective amount of a permeation enhancer that facilitates penetration of the formulation components through cell membranes, tissues, and extra-cellular matrices, including the cornea. The “effective amount” of the permeation enhancer represents a concentration that is sufficient to provide a measurable increase in penetration of one or more of the formulation components through membranes, tissues, and extracellular matrices as just described. Suitable permeation enhancers include, by way of example, methylsulfonylmethane (MSM; also referred to as methyl sulfone), combinations of MSM with dimethylsulfoxide (DMSO), or a combination of MSM and, in a less preferred embodiment, DMSO, with MSM particularly preferred.

### Kits and Packages

Provided herein are also kits and packages that include a compound of the disclosure, a retinal imaging device, and optionally suitable packaging. In one embodiment, a kit further includes instructions for use.

The retinal imaging device may include lens(es) and image sensors (thus forming a suitable retina scanner) for detecting a signal emitted. In some embodiments, the retinal imaging device detects a fluorescent signal. In some embodiments, the retinal imaging device further includes a laser light source which can be used to activate the fluorescent signal.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Tau Protein Retinal Staining

Retinas of different age of three-repeat tau (“3RTau”) mice (3 months, 6 months and 12 months) were mounted on slides. The retina were washed with PBS 2 times for 5 minutes. A solution of 98% formic acid was added—5 minutes for antigen retrieval. The sampled were washed with distilled water—2 times for 5 minutes. The samples were equilibrated in 1×PBS for 15 minutes, then blocked with 10% goat/donkey serum in 1×PBST (depending on antibodies) for 1 hour. The samples were then incubated with three-repeat tau antibody in 10% goat serum in 1×PBST at 4 degree C. overnight, then rinsed with 1×PBST 3 times for 5 minutes each. Samples were incubated with secondary antibody (Alexa Fluor 568 Anti mouse) in 1×PBST for 1 hour, then covered with foil, then rinsed with 1×PBST 3 times for 5 minutes each. Samples were stained with Compound 5 working solution for 30 minutes at room temperature, covered in foil, and washed with 1×PBS 3 times for 5 minutes each. Samples were stained with DAPI (300 nM or 100 ng/mL) in dark for 10 minutes, then tissues were washed 3×10 minutes with PBS. Antifade DAKO mounting medium was added, and coverslip and kept under foil until imaging. Images were obtained using a Leica confocal microscope.

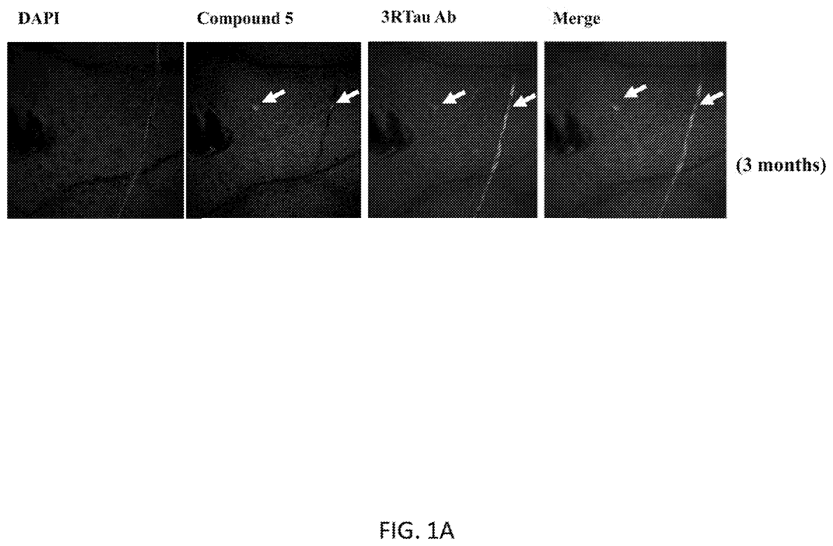

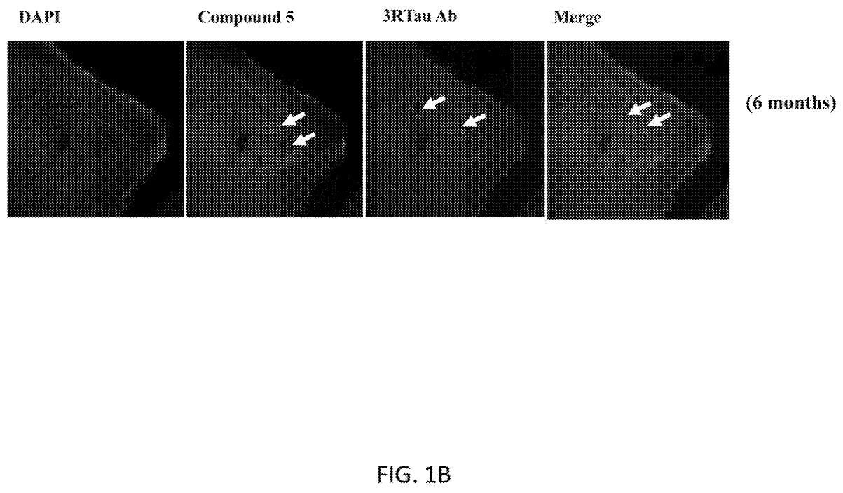

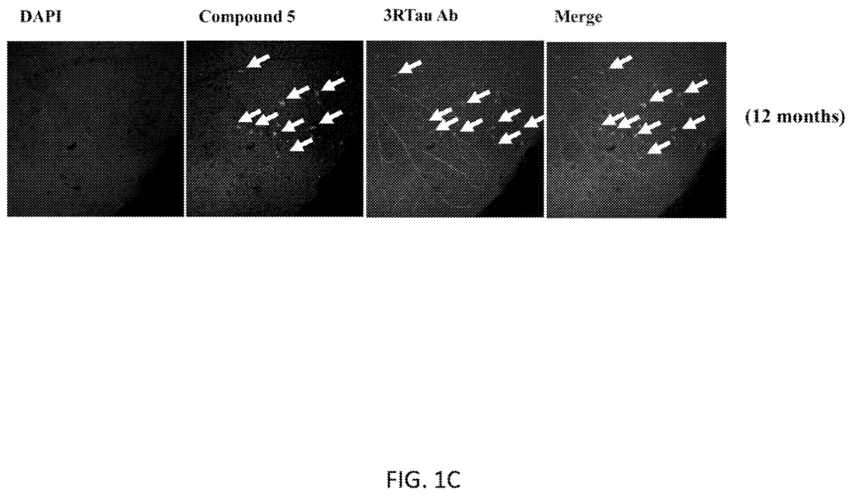

Hyperphosphorylated 3RTau was detected in the retina of different ages of 3RTau transgenic mice. FIGS. 1A, 1B, and 1C show the appearance of fluorescent aggregates (˜5-10 μm in diameter) in the retinal sections of 12 month old 3RTau mice, which were absent in control retinal sections. Immunofluorescence with the 3RTau antibody shows the presence of accumulated tau aggregates in blood vessels and neurons of 3RTau mice, as expected. Merged images show the colocalization of Compound 5 with the 3RTau antibody in the cell body of neurons. Analysis using Image J to assess the degree of colocalization and statistical testing of the relationship revealed a Pearson correlation coefficient (PCC) of 0.67 for the 3RTau mouse, compared to 0.04 for the control mouse (PCC measures the correlation between the pixel values for two reporter channels, where −1=complete anti-colocalization; 0=non colocalization; 1=complete colocalization).

With increase in age, immunoreactivity with 3RTau Ab in retina increases. In 12 months transgenic mice 3Rtau staining was robust as compared to 6 months and 3 month transgenic mice. The 3Rtau immunoreactivity in retina colocalizes with Compound 5. Colocalization of the Compound 5 and 3RTau antibody was observed by immunostaining confirming specificity for tau. Interestingly, at 3 months, tau aggregates were observed closer to the optic disc, whereas at 12 months, the increase in tau was more prevalent towards the periphery of the retina. In wild type mice of comparable age, no immunostaining with 3RTau Ab was detected. In conclusion, the study indicated that Compound 5 has a possibility of using as a diagnostic agent for detecting hyperphosphorylated tau in retina. The results shown in FIGS. 1A, 1B, and 1C provide evidence that compounds described herein have the potential to diagnose Alzheimer's and other tauopathies and to monitor disease progression.

### Example 2: Imaging

Fluorescent imaging study was conducted on a Leica DMI 4000B microscope (Leica, Germany) equipped with a TCS SPE camera and Leica 10, 20 and 40× objectives. The following lasers were used to visualize fluorescent probes pertaining to DAPI (blue, nuclear stain), Compound 5 (green) and 3RTau (red): 408, 488 and 568 nm. Z-stacked images were taken at 40× at 0.5 μm increments to visualize the entire thickness of the tissue.

FIGS. 1A, 1B, and 1C illustrate retinal sections of 3RTau mice of different ages stained with 3RTau Ab, Compound 5 and DAPI, at 3 months (FIG. 1A), 6 months (FIG. 1B), and 12 months (FIG. 1C).

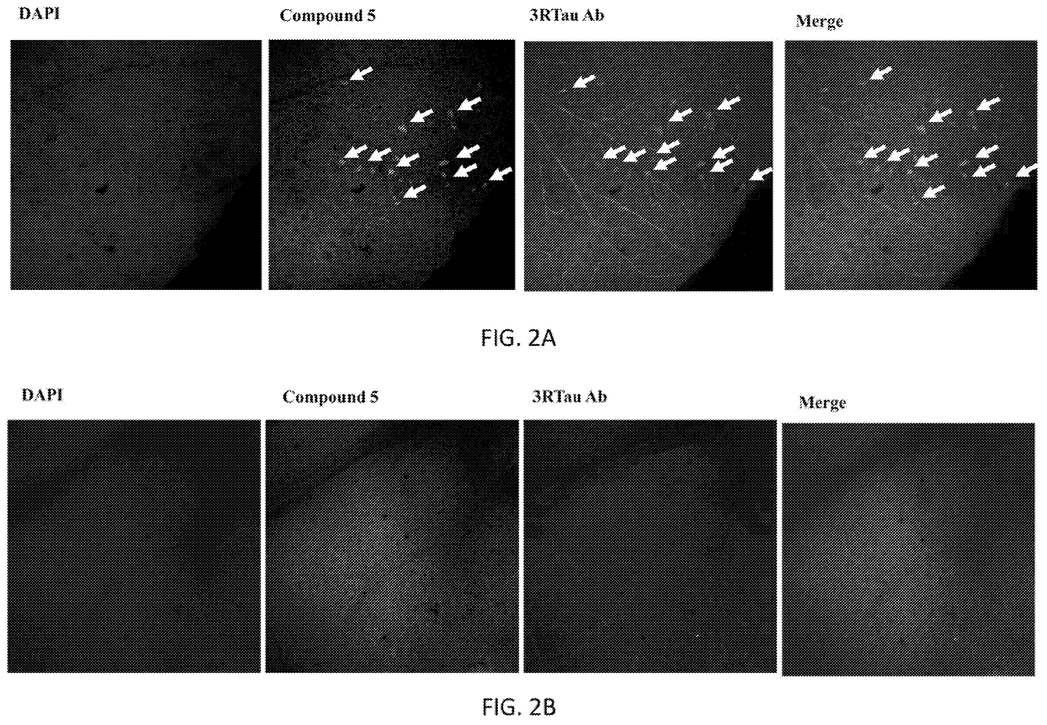

FIGS. 2A and 2B illustrate 3RTau mice (12 months) retinal section stained with 3RTau Ab, Compound 5 and DAPI (FIG. 2A) and wild type mice (12 months) retinal section stained with 3RTau Ab, Compound 5 and DAPI (FIG. 2B).

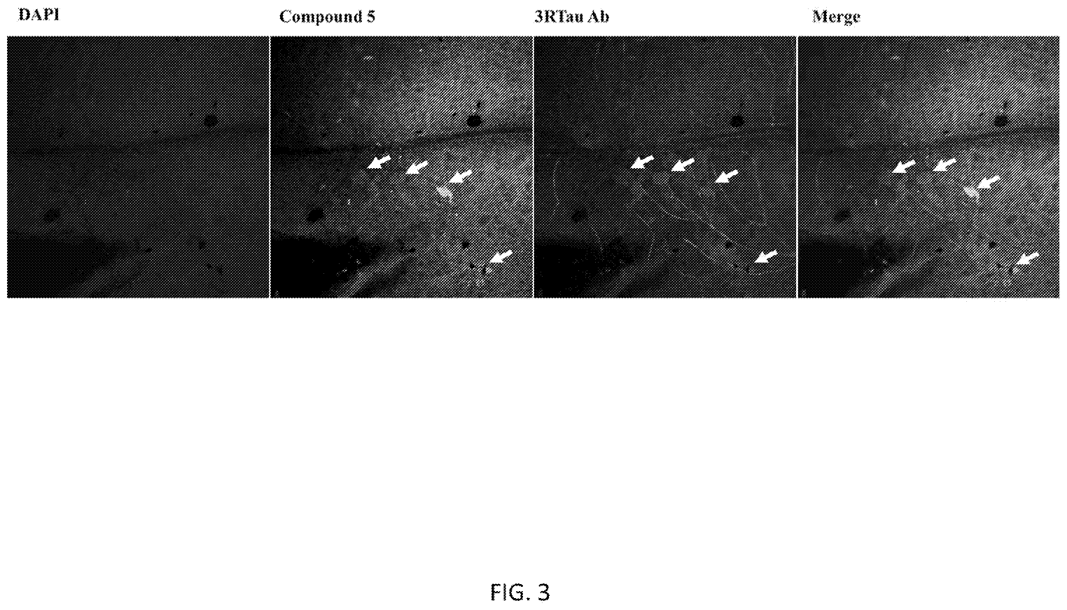

FIG. 3 illustrates 3RTau mice (12 months) retinal section stained with 3RTau Ab, Compound 5 and DAPI.

### Example 3: Synthesis

Synthesis of Compound 23

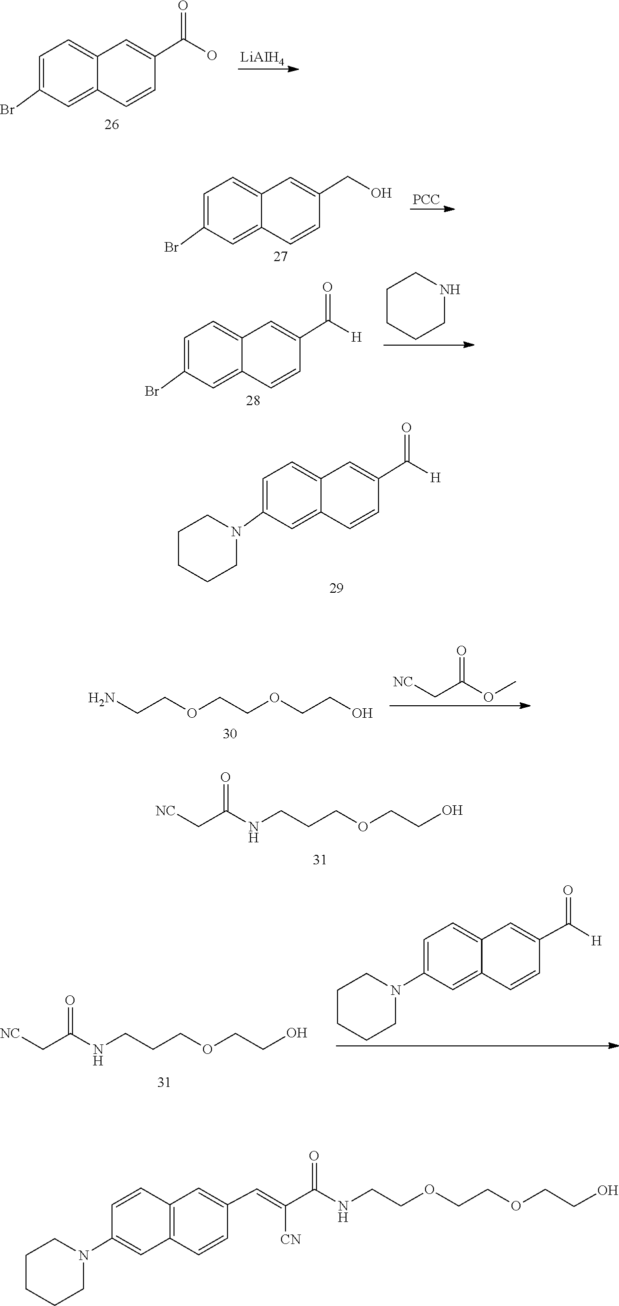

### Synthesis of (6-bromonaphthalen-2-yl)methanol (27)

To a solution of LiAlH₄ (8.2 g, 217 mmol in 500 mL THF) at 0° C. under N₂, a solution of methyl 6-bromo-2-naphthoate (26) (50.0 g, 189 mmol) in 500 mL of anhydrous THF was added dropwise. The reaction mixture was left stirring for 1 h at 0° C. Upon reaction completion monitored by TLC, the mixture was treated with H₂O, 15% NaOH, H₂O (1:1:3, v/v/v). After filtration, the filtrate was concentrated and extracted with EA, dried over NaSO₄. The crude product was purified from (PE:EA=3:1) to obtain the title compound.

### Synthesis of 6-bromo-2-naphthaldehyde (28)

In a suspension of (6-bromonaphthalen-2-yl)methanol (27) (42.0 g, 177 mmol) and silica gel (76.4 g) in DCM (500 mL), was added PCC (76.4 g, 354 mmol). The reaction was stirred at RT (room temperature, 25±5° C.) for 1.5 h. Upon completion, it was filtered through a pad of silica and concentrated under reduced pressure to obtain the title compound.

### Synthesis of 6-(piperidin-1-yl)-2-naphthaldehyde (29)

In dry and degassed toluene (300 mL), were added Pd(OAc)₂ (1.5 g, 6.3 mmol), BINAP (4.4 g, 7.1 mmol), 6-bromo-2-naphthaldehyde (28) (30.0 g, 127.8 mmol), Cs₂CO₃ (60.0 g, 183.9 mmol) and piperidine (12.7 g, 149.5 mmol). The reaction was left stirring for 8 h at 115° C. After cooling, the mixture was filtered and washed with EA, then concentrated to a third of the volume, to which was added 200 ml of 6 N hydrochloric acid with full stirring. Water phase was separated and extracted with DCM three times, then adjusted to alkaline with 5 N NaOH and extracted with EA. The organic phase was concentrated to yield crude material, which was further purified via silica gel chromatography (PE:EA=20:1 to 2:1) to obtain the title compound.

### Synthesis of 2-cyano-N-(2-(2-(2-hydroxyethoxy)ethoxy)ethyl)acetamide (31)

In a pear-shaped flask, 30 (6.0 g, 40 mmol) was added to methyl 2-cyanoacetate (4.0 g, 40 mmol) with stirring. The mixture was let stirring overnight at RT and concentrated to a crude material. The crude material was directly used in the next step.

### Synthesis of (E)-2-cyano-N-(2-(2-(2-hydroxyethoxy)ethoxy)ethyl)-3-(6-(piperidin-1-yl)naphthalen-2-yl)acrylamide (Compound 23)

To a solution of 6-(piperidin-1-yl)-2-naphthaldehyde (29) (7.0 g, 29.3 mmol) and 2-cyano-N-(2-(2-(2-hydroxyethoxy)ethoxy)ethyl)acetamide (31) (7.9 g, 36.5 mmol) in anhydrous THF (250 mL) was added piperidine (0.5 g, 5.9 mmol) and the resulting mixture was refluxed for 12 h. The reaction was then concentrated under reduced pressure to a crude material, which was purified via silica gel chromatography to obtain the title compound. Exact Mass 437.23; m/e 437.23 (100%), 438.23 (28.9%), 439.24 (4.7%); elemental analysis (C₂₅H₃₁N₃O₄): C, 68.63%, H, 7.14%, N, 9.60%, O, 14.63%.

Synthesis of Compound 24

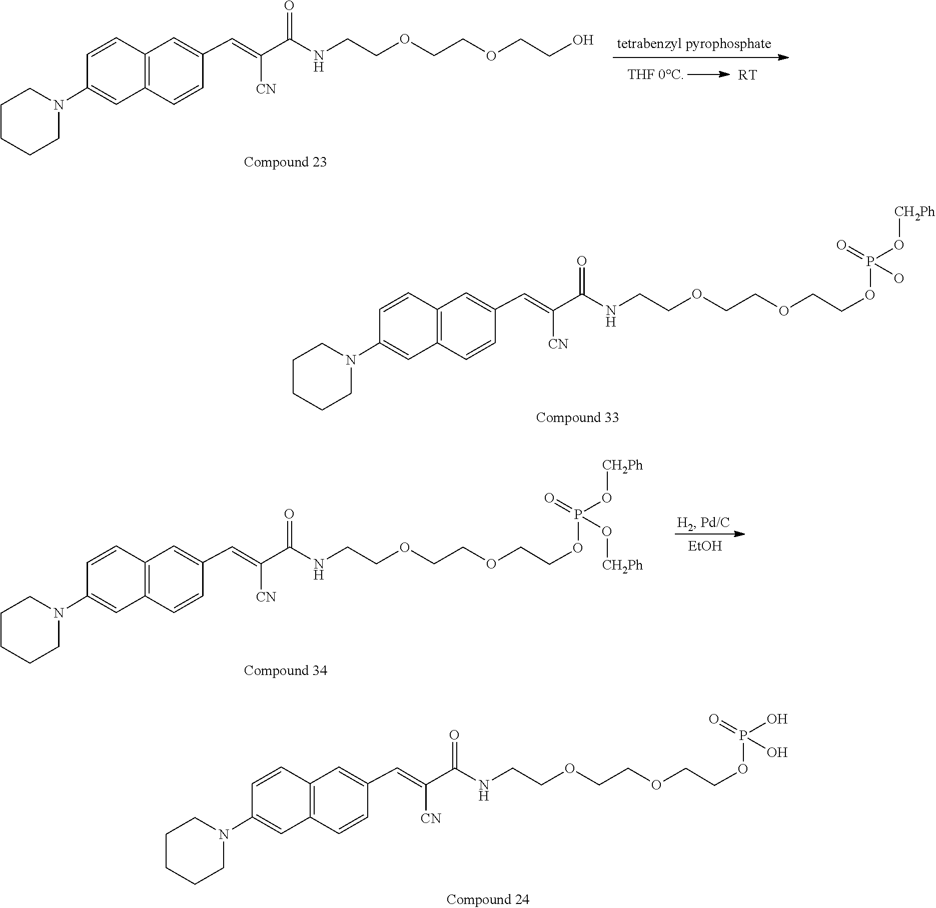

Synthesis of 33

To 1 g (2.3 mM) of Compound 23 in 15 mL of THF at zero degrees was added 1.6 g (3 mM) of tetrabenzyl pyrophosphate prepared using Merck Organic Synthesis preparation, resulting in a deep red solution. Sodium hydride (100 mg, 2.5 mM, 60% in oil) was added. After 15 min and warming to room temperature a solid started precipitating. DMF (5 mL) was added and stirred at RT for 1 hr. Water (200 mL) and ethyl acetate (200 mL) were added. The ethyl acetate layer was dried and evaporated. Purification by ISCO® on an 80 g silica gel cartridge using 0-100% hexane/ethyl acetate afforded the title compound. A ¹H NMR spectrum of Compound 8 is presented in FIGS. 1A to 1C. MS (m/z) 701.3 [M+H]t

Synthesis of Compound 24

To 1 gram of (Compound 33), 95% ethanol (150 mL), degassed with argon was added. 120 mg of 10% Pd/C was added and hydrogen gas was bubbled into reaction mixture for 5 min. The mixture was then stirred under a hydrogen balloon for 3 hrs. Reaction mixture was degassed with argon and evaporated. Purification by prep LC/MS on a C18 column 25×250 mm using 0 to 100% water containing 2 g per liter ammonium acetate. After free drying, Compound 24 was obtained. A ¹H NMR spectrum (D₂O) of Compound 9 is presented in FIG. 2A and FIG. 2B.

Synthesis of Compound 25

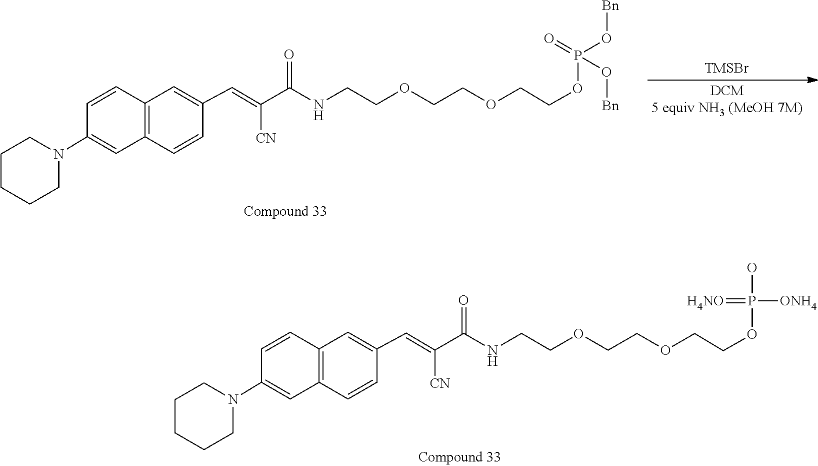

A solution of Compound 33 (5 mmol, 3.49 g, 1 equiv) in anhydrous CH₂Cl₂ (100 ml) was cooled to 0° C. under Argon, and trimethylsilyl bromide (50 mmol, 6.8 ml, 10 equiv) was added via syringe. The reaction mixture was stirred at 0° C. for 30 mins and completion of conversion was monitored by HPLC. The mixture was then quenched with MeOH (˜50 mL) and stirred for 10 minutes. The solution was evaporated to dryness; this step was repeated four times.

The organic solvents were evaporated under vacuum, the residue was suspended in trace amount of MeOH, and EtOAc was added to induce precipitation of the phosphonic acid. The residue was filtered and washed with EtOAc (×2). The residue was then dried under vacuum to give the desired phosphate acid.

Treatment of the phosphonic acid with NH₄OAc (25 mmol, 1.93 g, 5 equiv) and 150 ml water at room temperature and kept stirring for another 15 mins to give a clear red/orange solution. The reaction mixture was then lyophilized to give the final product. LC-MS: (ES, m/z) 518 [M+1]⁺. 1H-NMR: (400 MHz, CD₃OD) δ 8.31-8.22 (m, 2H), 8.08 (dd, J=8.8, 1.9 Hz, 1H), 7.81 (d, J=9.2 Hz, 1H), 7.74 (d, J=8.8 Hz, 1H), 7.40 (dd, J=9.2, 2.5 Hz, 1H), 7.18 (d, J=2.5 Hz, 1H), 4.08-3.98 (m, 2H), 3.79-3.65 (m, 8H), 3.58 (t, J=5.5 Hz, 2H), 3.45-3.39 (m, 4H), 1.86-1.63 (m, 6H).

### Example 4: In Vivo Assay

Experimental Design: In vivo fluorescence retinal imaging experiments are performed on ketamine anesthetized mice as indicated in the Table:

| Mouse | Disease | N per formulation | |
|---|---|---|---|
| Type | Model | Formulation | Vehicle |
| 3RTau | AD | 5 | 5 |
| 4RTau | AD | 5 | 5 |
| WT | Control | 5 | 5 |

Since the balance of 3R and 4R tau isoforms is affected in a multitude of tauopathy diseases including AD, both 3RTau and 4RTau tg models will be used to explore the interaction of compounds described herein that fluoresce with either variant of tau. Female tg mice may be employed since they have been reported to develop amyloid plaques faster than males. Real time in vivo fluorescence imaging of the retinas will be captured using a Phoenix Micron IV imaging system. Increasing IV doses, selected from pilot pharmacokinetic studies with biocompatible formulations of compounds described herein will be given to mice and retinal fluorescence determined at different times post-dose. Systemic and retinal concentrations will be determined in satellite animals (WT control strains) to understand the relationship between dose, concentration, and response. Mice will be sacrificed for ex vivo brain and retina analysis after live imaging is completed to confirm tau presence via immunofluorescence (IF) microscopy with antibodies that recognize total tau, hyperphosphorylated tau, and tau conformation epitopes (3RTau and 4RTau).

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for determining whether a patient suffers from a neurological disease or disorder, comprising detecting the presence of a phosphorylated tau protein in a tissue of the patient, wherein the detecting comprises contacting the phosphorylated tau protein with a compound of formula Ic: 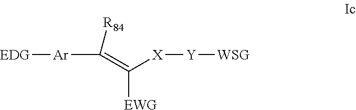
wherein
EDG is:
a) heterocycloalkyl of no more than 10 carbons optionally substituted with one or more R₁₇; or
b) —NR₁₀R₁₁;
wherein each R₁₇ is independently halogen, —ORB, —NR₁₉R₂₀, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;
each of R₁₀, R₁₁, R₁₈, R₁₉ and R₂₀ is independently hydrogen, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, each of which except for hydrogen is optionally substituted with one or more R₂₁;
each of R₂₁ is independently halogen, —OR₂₂, —NR₂₃R₂₄, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, wherein the alkyl, heteroalkyl, cycloalkyl, heterocycloalkyl, arylene or heteroarylene is optionally substituted with one or more R₂₅;
each of R₂₂, R₂₃ and R₂₄ is independently hydrogen or C₁-C₁₀ alkyl; and
each of R₂₅ is independently C₁-C₁₀ alkyl, C₁-C₁₀ heteroalkyl, cycloalkyl of no more than 10 carbons, heterocycloalkyl of no more than 10 carbons, arylene of no more than 10 carbons or heteroarylene of no more than 10 carbons;
Ar is arylene of no more than 14 carbon atoms or heteroarylene of no more than 14 carbon atoms, each optionally substituted with one or more R₁;
each R₁ is independently halogen, —OR₂, —NR₃R₄, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons wherein the alkyl, heteroalkyl, cycloalkyl, heterocycloalkyl, arylene, or heteroarylene is optionally substituted with one or more R₅,
R₂, R₃ and R₄ are independently hydrogen, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, each of which except for hydrogen is optionally substituted with one or more R₅;
each R₅ is independently halogen, —OR₆, —NR₇R₈, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;
R₆, R₇, R₈ and R₈₄ are independently hydrogen or C₁-C₁₀ alkyl;
EWG is selected from a group consisting of —F, —Cl, —Br, —CH═O, NO₂, —CF₃, —CCl₃, —SO₃H and —CN;
WSG is:
i) 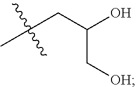 [SMILES: *CC(O)CO]
ii) polyethylene glycol, polypropylene glycol, co-polymer of polyethylene glycol and polypropylene glycol, or alkoxy derivatives thereof;
iii) 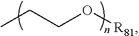
wherein n is an integer from 1-50 and R₈₁ is hydrogen, a C₁-C₁₀ alkyl, a C₁-C₁₀ alkenyl, or a C₁-C₁₀ alkynyl, wherein the alkyl, alkenyl, or alkynyl is optionally substituted with one or more C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;
iv) 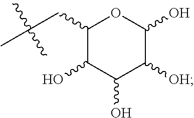 [SMILES: *CC1OC(O)C(O)C(O)C1O]
v) 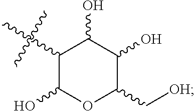
vi)
—(C₁-C₁₀ alkyl)-R₃₃-R₃₇, wherein:
R₃₃ is heteroarylene of no more than 10 carbons; and
R₃₇ is —(C₁-C₆ alkyl) (heterocycloalkyl of no more than 10 carbons);
vii) 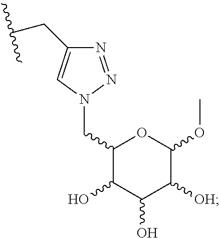 [SMILES: *Cc1cn(CC2OC(OC)C(O)C(O)C2O)nn1]
viii)
—(C₁-C₁₀ heteroalkyl)-R₃₃-R₃₇, wherein:
R₃₃ is heteroarylene of no more than 10 carbons; and
R₃₇ is —(C₁-C₆ alkyl) (heterocycloalkyl of no more than 10 carbons); or
ix) 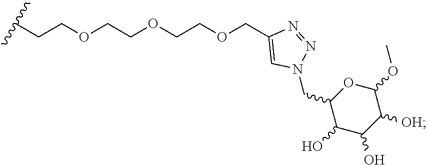 [SMILES: *CCOCCOCCOCc1cn(CC2OC(OC)C(O)C(O)C2O)nn1]
X is C═O or SO₂ or X and R₈₄ join to form a pyridinyl; and
Y is NH or S.

2. The method of claim 1, wherein the contacting is in vivo.

3. The method of claim 1, wherein the tissue is an eye tissue.

4. The method of claim 1, wherein the contacting, upon activation by a light, causes emission of detectable signal.

5. The method of claim 4, wherein the detectable signal is a fluorescent signal.

6. The method of any preceding claim, wherein the neurological disease or disorder is Alzheimer's disease or Chronic Traumatic Encephalopathy (CTE).

7. The method of claim 1, wherein the neurological disease or disorder is selected from an age-related disease or disorder, a genetic disease or disorder, an injury-related disease or disorder, and a psychiatric disease or disorder.

8. The method of claim 7, wherein the age-related disease or disorder is selected from Parkinson's dementia, vascular dementia, and Amyotrophic lateral sclerosis, wherein the genetic disease or disorder is Down syndrome, wherein the injury-related disease or disorder is selected from Traumatic Brain Injury and Chronic Traumatic Encephalopathy, and wherein the psychiatric disease or disorder is selected from schizophrenia and depression.

9. The method of claim 1, wherein the phosphorylated tau protein includes at least three moles of phosphate per mole of protein.

10. The method of claim 1, wherein the phosphorylated tau protein is a three-repeat tau, a four-repeat tau, or a combination thereof.

11. A method for preparing a patient for diagnosis of a neurological disease or disorder, comprising administering to a tissue of the patient a compound of formula Ic: 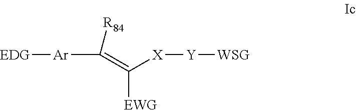
wherein
EDG is:
a) heterocycloalkyl of no more than 10 carbons optionally substituted with one or more R₁₇; or
b) —NR₁₀R₁₁;
wherein each R₁₇ is independently halogen, —ORB, —NR₁₉R₂₀, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;
each of R₁₀, R₁₁, R₁₈, R₁₉ and R₂₀ is independently hydrogen, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, each of which except for hydrogen is optionally substituted with one or more R₂₁;
each of R₂₁ is independently halogen, —OR₂₂, —NR₂₃R₂₄, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, wherein the alkyl, heteroalkyl, cycloalkyl, heterocycloalkyl, arylene or heteroarylene is optionally substituted with one or more R₂₅;
each of R₂₂, R₂₃ and R₂₄ is independently hydrogen or C₁-C₁₀ alkyl; and
each of R₂₅ is independently C₁-C₁₀ alkyl, C₁-C₁₀ heteroalkyl, cycloalkyl of no more than 10 carbons, heterocycloalkyl of no more than 10 carbons, arylene of no more than 10 carbons or heteroarylene of no more than 10 carbons;
Ar is arylene of no more than 14 carbon atoms or heteroarylene of no more than 14 carbon atoms, each optionally substituted with one or more R₁;
each R₁ is independently halogen, —OR₂, —NR₃R₄, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons wherein the alkyl, heteroalkyl, cycloalkyl, heterocycloalkyl, arylene, or heteroarylene is optionally substituted with one or more R₅,
R₂, R₃ and R₄ are independently hydrogen, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons, each of which except for hydrogen is optionally substituted with one or more R₅;
each R₅ is independently halogen, —OR₆, —NR₇R₈, C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;
R₆, R₇, R₈ and R₈₄ are independently hydrogen or C₁-C₁₀ alkyl;
EWG is selected from a group consisting of —F, —Cl, —Br, —CH═O, NO₂, —CF₃, —CCl₃, —SO₃H and —CN;
WSG is:
i) 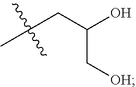 [SMILES: *CC(O)CO]
ii) polyethylene glycol, polypropylene glycol, co-polymer of polyethylene glycol and polypropylene glycol, or alkoxy derivatives thereof;
iii) 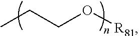
wherein n is an integer from 1-50 and R₈₁ is hydrogen, a C₁-C₁₀ alkyl, a C₁-C₁₀ alkenyl, or a C₁-C₁₀ alkynyl, wherein the alkyl, alkenyl, or alkynyl is optionally substituted with one or more C₁-C₁₀ alkyl, C₁-C₁₀ heteroalkyl, cycloalkyl of no more than 10 carbons, heterocycloalkyl of no more than 10 carbons, arylene of no more than 10 carbons, or heteroarylene of no more than 10 carbons;
iv) 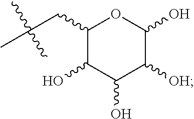 [SMILES: *CC1OC(O)C(O)C(O)C1O]
v) 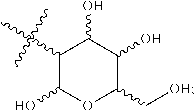
vi)
—(C₁-C₁₀ alkyl)-R₃₃-R₃₇, wherein:
R₃₃ is heteroarylene of no more than 10 carbons; and
R₃₇ is —(C₁-C₆ alkyl) (heterocycloalkyl of no more than 10 carbons);
vii) 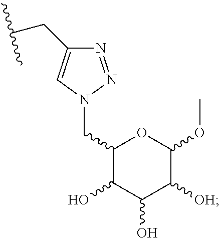 [SMILES: *Cc1cn(CC2OC(OC)C(O)C(O)C2O)nn1]
viii)
—(C₁-C₁₀ heteroalkyl)-R₃₃-R₃₇, wherein:
R₃₃ is heteroarylene of no more than 10 carbons; and
R₃₇ is —(C₁-C₆ alkyl) (heterocycloalkyl of no more than 10 carbons); or
ix) 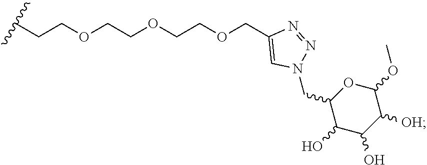 [SMILES: *CCOCCOCCOCc1cn(CC2OC(OC)C(O)C(O)C2O)nn1]
X is C═O or SO₂ or X and R₈₄ join to form a pyridinyl; and
Y is NH or S.

12. The method of claim 11, further comprising detecting the binding of the compound of formula Ic to a phosphorylated tau protein.

13. The method of claim 12, wherein the binding, upon activation by a light, causes emission of a detectable signal.

14. The method of claim 13, wherein the signal is a fluorescent or infrared signal.

15. The method of claim 11, wherein the administration is intravenous administration.

16. The method of claim 11, wherein the administration is localized at the retina of the eye.

17. The method of claim 11, wherein the administration is topical administration.

18. The method of any one of claims 11-17, wherein the neurological disease or disorder is Alzheimer's disease or Chronic Traumatic Encephalopathy (CTE).

19. The method of claim 11, wherein the neurological disease or disorder is selected from an age-related disease or disorder, a genetic disease or disorder, an injury-related disease or disorder, and a psychiatric disease or disorder.

20. The method of claim 19, wherein the age-related disease or disorder is selected from Parkinson's dementia, vascular dementia, and Amyotrophic lateral sclerosis, wherein the genetic disease or disorder is Down syndrome, wherein the injury-related disease or disorder is selected from Traumatic Brain Injury and Chronic Traumatic Encephalopathy, and wherein the psychiatric disease or disorder is selected from schizophrenia and depression.

21. The method of claim 12, wherein the phosphorylated tau protein includes at least three moles of phosphate per mole of protein.

22. The method of claim 12, wherein the phosphorylated tau protein is a three-repeat tau, a four-repeat tau, or a combination thereof.
